(19) United States
(12) Patent Application Publication
(10) Pub. No.: US 2008/0090822 A1
(43) Pub. Date: Apr. 17, 2008

(54) Piperazine Derivatives As Glyt 1 Inhibitors

(75) Inventors: Daniel Marcus Bradley, Essex (GB); Clive Leslie Branch, Essex (GB); Bethany Joy Brown, Hertfordshire (GB); Wai Ngor Chan, Essex (GB); Steven Coulton, Essex (GB); Martin Leonard Gilpin, Essex (GB); Sharon Lisa Gough, Hertfordshire (GB); Jacqueline Anne MacRitchie, Essex (GB); Howard Robert Marshall, Essex (GB); David John Nash, Essex (GB); Roderick Alan Porter, Essex (GB); Anthony William Dean, Hertfordshire (GB); Paul Martin Doyle, Essex (GB); Brian Evans, Hertfordshire (GB); Luigi Piero Stasi, Verona (IT)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Glaxo Group Limited

(21) Appl. No.: 11/908,149

(22) PCT Filed: Mar. 9, 2006
(86) PCT No.: PCT/EP06/02485

§ 371 Date: Sep. 10, 2007

(30) Foreign Application Priority Data
Mar. 11, 2005 (GB) 0505086.9

(51) Int. Cl. A61K 31/535; A61K 31/497; A61P 25/18; C07D 401/02; C07D 401/14

(52) U.S. Cl. 514/235.8; 514/253.01; 544/121; 544/360; 544/364

(57) ABSTRACT

The invention provides a compound of formula (I) or a salt or solvate thereof: wherein R¹, n, X, Y and Z are as defined in the specification, and uses of such compounds. The compounds inhibit GlyT1 transporters and are useful in the treatment of certain neurological and neuropsychiatric disorders, including schizophrenia.

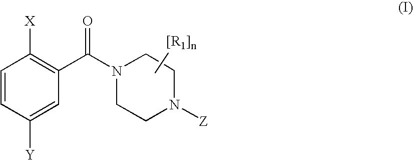

The present invention relates to glycine transporter inhibiting compounds, their use in the manufacture of medicaments for treating neurological and neuropsychiatric disorders, in particular psychoses, dementia or attention deficit disorder. The invention further comprises processes to make these compounds and pharmaceutical formulations thereof.

Molecular cloning has revealed the existence in mammalian brains of two classes of glycine transporters, termed GlyT1 and GlyT2. GlyT1 is found predominantly in the forebrain and its distribution corresponds to that of glyinergic pathways and NMDA receptors (Smith, et al., Neuron, 8, 1992: 927-935). Molecular cloning has further revealed the existence of three variants of GlyT1, termed GlyT-1a, GlyT-1b and GlyT-1c (Kim et al., Molecular Pharmacology, 45, 1994: 608-617), each of which displays a unique distribution in the brain and peripheral tissues. The variants arise by differential splicing and exon usage, and differ in their N-terminal regions. GlyT2, in contrast, is found predominantly in the brain stem and spinal cord, and its distribution corresponds closely to that of strychnine-sensitive glycine receptors (Liu et al., J. Biological Chemistry, 268, 1993: 22802-22808; Jursky and Nelson, J. Neurochemistry, 64, 1995: 1026-1033). Another distinguishing feature of glycine transport mediated by GlyT2 is that it is not inhibited by sarcosine as is the case for glycine transport mediated by GlyT1. These data are consistent with the view that, by regulating the synaptic levels of glycine, GlyT1 and GlyT2 selectively influence the activity of NMDA receptors and strychnine-sensitive glycine receptors, respectively.

NMDA receptors are critically involved in memory and learning (Rison and Staunton, Neurosci. Biobehav. Rev., 19 533-552 (1995); Danysz et al, Behavioral Pharmacol., 6 455-474 (1995)); and, furthermore, decreased function of NMDA-mediated neurotransmission appears to underlie, or contribute to, the symptoms of schizophrenia (Olney and Farber, Archives General Psychiatry, 52, 998-1007 (1996). Thus, agents that inhibit GlyT1 and thereby increase glycine activation of NMDA receptors can be used as novel antipsychotics and anti-dementia agents, and to treat other diseases in which cognitive processes are impaired, such as attention deficit disorders and organic brain syndromes. Conversely, over-activation of NMDA receptors has been implicated in a number of disease states, in particular the neuronal death associated with stroke and possibly neurodegenerative diseases, such as Alzheimer's disease, multi-infarct dementia, AIDS dementia, Huntington's disease, Parkinson's disease, amyotrophic lateral sclerosis or other conditions in which neuronal cell death occurs, such as stroke or head trauma. Coyle & Puttfarcken, Science, 262, 689-695 (1993); Lipton and Rosenberg, New Enql. J. of Medicine, 330, 613-622 (1993); Choi, Neuron, 1, 623-634 (1988). Thus, pharmacological agents that increase the activity of GlyT1 will result in decreased glycine-activation of NMDA receptors, which activity can be used to treat these and related disease states. Similarly, drugs that directly block the glycine site of the NMDA receptors can be used to treat these and related disease states. Glycine transport inhibitors are already known in the art, for example as disclosed in published international patent application WO03/055478 (SmithKline Beecham).

However, there still remains the need to identify further compounds that can inhibit GlyT1 transporters, including those that inhibit GlyT1 transporters selectively over GlyT2 transporters.

International patent application WO97/28128 (Zeneca Limited) discloses certain pyridinyl, pyridazinyl, pyrimidinyl and triazinyl derivatives which are claimed to inhibit the enzyme oxido squalene cyclase. European patent application EP1247809 (Pfizer Products Inc) discloses certain triazine derivatives as being useful as sorbitol dehydrogenase inhibitors.

It has now been found that a novel class of compounds inhibit GlyT1 transporters and are thus useful in the treatment of certain neurological and neuropsychiatric disorders, including schizophrenia.

Thus, in the first aspect, there is provided a compound of formula (I) or a salt or solvate thereof: 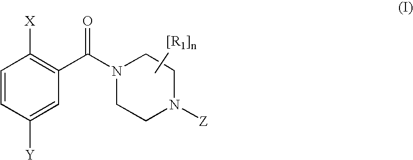
wherein
X is —NR₃R₄, wherein
   R₃ and R₄ are independently selected from hydrogen and C₁₋₆alkyl, or R₃ and R₄, together with the nitrogen atom to which they are attached, form an N-linked 3- to 7-membered monocyclic heterocyclic ring or an 8- to 11-membered bicyclic heterocyclic ring, which ring optionally comprises one or more further heteroatoms selected from N, O and S; and which C₁₋₆alkyl group or ring is optionally substituted by one or more groups selected from halo, C₁₋₄alkyl, C₁₋₄haloalkyl, C₁₋₄alkoxy, C₁₋₄haloalkoxy, C₁₋₄alkylthio, halo and hydroxy;
Y is S(O)ₘR₅ or —SO₂NHR₆ wherein
   m is 1 or 2; and
   R₅ is selected from C₁₋₆alkyl, C₃₋₇cycloalkyl, C₅₋₁₁aryl and C₄₋₁₀heteroaryl, which C₁₋₆alkyl, C₃₋₇cycloalkyl, C₅₋₁₁aryl or C₄₋₁₀heteroaryl is optionally substituted with one or two groups selected from halo, C₁₋₄alkoxy and C₁₋₄haloalkoxy;
   R₆ is C₁₋₆alkyl; which C₁₋₆alkyl is optionally substituted with one or more groups selected from halo, C₁₋₄alkoxy and C₁₋₄haloalkoxy;
n is 0, 1 or 2,
each R₁ is independently selected from C₁₋₆alkyl, halo, C₁₋₆haloalkyl C₁₋₄alkoxy and C₁₋₄haloalkoxy;
Z is an optionally substituted phenyl group Z′:

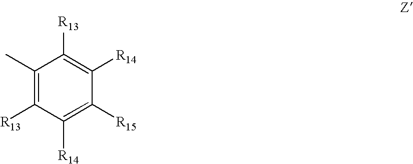

wherein each R₁₃ is independently selected from hydrogen, halogen, hydroxy, cyano, amino, nitro, C₁₋₆alkyl, haloC₁₋₄alkyl, haloC₁₋₄alkoxy, C₆₋₁₁arylC₁₋₄alkoxy, C₁₋₄alkylthio, hydroxyC₁₋₄alkyl, C₁₋₄alkoxyC₁₋₄alkyl, C₃₋₆cycloalkylC₁₋₄alkyl, C₃₋₆cycloalkyl, C₃₋₆cycloalkylC₁₋₄alkoxy, C₁₋₄alkanoyl, C₁₋₄alkoxycarbonyl, C₁₋₄alkoxycarbonylC₁₋₄alkyl, C₁₋₄alkylsulfonyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylamido, C₁₋₄alkylsulfonamidoC₁₋₄alkyl, C₁₋₄alkylamidoC₁₋₄alkyl, C₆₋₁₁arylsulfonamido, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, C₁₋₄acyl, C₆₋₁₁arylC₁₋₄alkyl, C₁₋₄alkylaminoC₁₋₄alkyl, a group R_{9″}R_{10″}N—, R₉R₁₀NCO(CH₂)ₚ, R_{9′}R_{10′}NSO₂(CH₂)ₚ or R_{9′}SO₂NR_{10′}(CH₂)ₚ, —CR_{9′}═NR_{10′}, —CR_{9′}═NOR_{10′}, —CR_{9′}═C(CN)₂, —CR_{9′}═CH(CN), R_{9′}R_{10′}N(CH₂)_{q}— and R_{9′}R_{10′}N(CH₂)_{q}O—, wherein
each R₉ and R₁₀ is independently C₁₋₄alkyl, or where appropriate R₉R₁₀ forms part of a C₃₋₆azacyloalkane or C₃₋₆(2-, 3- or 4-oxo)azacycloalkane ring
each R_{9′} and R_{10′} is independently selected from R₉ and R₁₀ and hydrogen;
each R_{9″} and R_{10″} is independently selected from R_{9′} and R_{10′} and C₁₋₄alkanoyl;
p is selected from 0, 1, 2, 3 or 4;
q is selected from 2, 3 or 4;
   wherein each R₁₄ is independently selected from hydrogen, halogen, hydroxy, cyano, amino, nitro, C₁₋₆alkyl, C₁₋₄alkoxy, haloC₁₋₄alkyl, haloC₁₋₄alkoxy, C₃₋₁₆arylC₁₋₄alkoxy, C₁₋₄alkylthio, C₃₋₆cycloalkylC₁₋₄alkyl, C₃₋₆cycloalkyl, C₃₋₆cycloalkylC₁₋₄alkoxy, C₁₋₄alkoxycarbonyl, C₁₋₄alkoxycarbonylC₁₋₄alkyl, C₁₋₄alkylsulfonyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₁₋₄alkylamido, C₁₋₄alkylsulfonamidoC₁₋₄alkyl, C₁₋₄alkylsulfinyl, C₁₋₄haloalkylsulfinyl, C₁₋₄alkylamidoC₁₋₄alkyl, C₆₋₁₁arylsulfonamido, C₄₋₉heteroarylsulfonyl, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, C₁₋₄acyl, C₆₋₁₁aryl, C₆₋₁₁arylC₁₋₄alkyl, C₁₋₄alkylaminoC₁₋₄alkyl, a group R_{9″}R_{10″}N—, R₉R₁₀NCO(CH₂)ₚ, R_{9′}R_{10′}NSO₂(CH₂)ₚ or R_{9′}SO₂NR_{10′}(CH₂)ₚ, —CR_{9′}═NR_{10′}, —CR_{9′}═NOR_{10′}, —CR_{9′}═C(CN)₂, —CR_{9′}═CH(CN), R_{9′}R_{10′}N(CH₂)_{q}— and R_{9′}R_{10′}N(CH₂)_{q}O—, wherein
each R₉ and R₁₀ is independently C₁₋₄alkyl, or where appropriate R₉R₁₀ forms part of a C₃₋₆azacyloalkane or C₃₋₆(2-, 3- or 4-oxo)azacycloalkane ring
each R_{9′} and R_{10′} is independently selected from R₉ and R₁₀ and hydrogen;
each R_{9″} and R_{10″} is independently selected from R_{9′} and R_{10′} and C₁₋₄alkanoyl;
p is selected from 0, 1, 2, 3 or 4;
q is selected from 2, 3 or 4;
   wherein each R₁₅ is independently selected from hydrogen, halogen, hydroxy, cyano, amino, nitro, C₁₋₆alkyl, C₁₋₄alkoxy, haloC₁₋₄alkyl, haloC₁₋₄alkoxy, C₆₋₁₁arylC₁₋₄alkoxy, C₁₋₄alkylthio, hydroxyC₁₋₄alkyl, C₁₋₄alkoxyC₁₋₄alkyl, C₁₋₄haloalkoxyC₁₋₄alkyl, C₃₋₆cycloalkylC₁₋₄alkyl, C₃₋₆cycloalkyl, C₃₋₆cycloalkylC₁₋₄alkoxy, C₁₋₄alkanoyl, C₁₋₄haloalkanoyl, C₁₋₄alkoxycarbonyl, C₁₋₄alkoxycarbonylC₁₋₄alkyl, C₁₋₄alkylsulfonyl, C₁₋₄haloalkylsulfonyl, C₁₋₄alkylsulfinyl, C₁₋₄haloalkylsulfinyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylamido, C₁₋₄alkylsulfonamidoC₁₋₄alkyl, C₁₋₄alkylamidoC₁₋₄alkyl, C₆₋₁₁arylsulfonamido, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, C₁₋₄acyl, C₆₋₁₁aryl, C₆₋₁₁arylC₁₋₄alkyl, C₁₋₄alkylaminoC₁₋₄alkyl, a group R_{9″}R_{10″}N—, R₉R₁₀NCO(CH₂)ₚ, R_{9″}R_{10′}NSO₂(CH₂)ₚ or R_{9′}SO₂NR_{10′}(CH₂)ₚ, —CR_{9′}═NR_{10′}, —CR_{9′}═NOR_{10′}, —CR_{9′}═C(CN)₂, —CR_{9′}═CH(CN), R_{9′}R_{10′}N(CH₂)_{q}— and R_{9′}R_{10′}N(CH₂)_{q}O—, wherein
each R₉ and R₁₀ is independently C₁₋₄alkyl, or where appropriate R₉R₁₀ forms part of a C₃₋₆azacyloalkane or C₃₋₆(2-, 3- or 4-oxo)azacycloalkane ring
each R_{9′} and R_{10′} is independently selected from R₉ and R₁₀ and hydrogen;
each R_{9″} and R_{10″} is independently selected from R_{9′} and R_{10′} and C₁₋₄alkanoyl;
p is selected from 0, 1, 2, 3 or 4;
q is selected from 2, 3 or 4;
   or Z is selected from a monocyclic or bicyclic heteroaryl group, which monocyclic heteroaryl group is or which bicyclic heteroaryl group optionally is substituted by one or more groups selected from amino, halogen, hydroxy, cyano, nitro, C₂₋₄alkyl, C₁₋₄alkoxy, haloC₁₋₄alkyl, haloC₁₋₄alkoxy, C₆₋₁₁arylC₁₋₄alkoxy, C₁₋₄alkylthio, hydroxyC₁₋₄alkyl, C₁₋₄alkoxyC₁₋₄alkyl, C₁₋₄haloalkoxyC₁₋₄alkyl, C₃₋₆cycloalkylC₁₋₄alkyl, C₃₋₆cycloalkyl, C₃₋₆cycloalkylC₁₋₄alkoxy, C₁₋₄alkanoyl, C₁₋₄haloalkanoyl, C₁₋₄alkoxycarbonyl, C₁₋₄alkoxycarbonylC₁₋₄alkyl, C₁₋₄alkylsulfonyl, C₁₋₄haloalkylsulfonyl, C₁₋₄alkylsulfinyl, C₁₋₄haloalkylsulfinyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylamido, C₁₋₄alkylsulfonamidoC₁₋₄alkyl, C₁₋₄alkylamidoC₁₋₄alkyl, C₆₋₁₁arylsulfonamido, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, C₁₋₄acyl, C₆₋₁₁aryl, C₆₋₁₁arylC₁₋₄alkyl, C₁₋₄alkylaminoC₁₋₄alkyl, a group R_{9″}R_{10″}N—, R₉R₁₀NCO(CH₂)ₚ, R_{9′}R_{10′}NSO₂(CH₂)ₚ or R_{9′}SO₂NR_{10′}(CH₂)ₚ, —CR_{9′}═NR_{10′}, —CR_{9′}═NOR_{10′}, —CR_{9′}═C(CN)₂, —CR_{9′}═CH(CN), R_{9′}R_{10′}N(CH₂)_{q}— and R_{9′}R_{10′}N(CH₂)_{q}O—, wherein
each R₉ and R₁₀ is independently C₁₋₄alkyl, or where appropriate R₉R₁₀ forms part of a C₃₋₆azacyloalkane or C₃₋₆(2-, 3- or 4-oxo)azacycloalkane ring
each R_{9′} and R_{10′} is independently selected from R₉ and R₁₀ and hydrogen;
each R_{9″} and R_{10″} is independently selected from R_{9′} and R_{10′} and C₁₋₄alkanoyl;
p is selected from 0, 1, 2, 3 or 4;
q is selected from 2, 3 or 4;

As used herein, the term “C₁₋₆alkyl” refers to a straight or branched alkyl which contains from one to six carbon atoms in all isomeric forms. Examples include methyl, ethyl, propyl, isopropyl, butyl, isobutyl, sec-butyl, tert-butyl, pentyl, neopentyl, sec-pentyl, n-pentyl, isopentyl, tert-pentyl and hexyl.

As used herein, the term “C₃₋₇cycloalkyl” refers to a non-aromatic cyclic saturated hydrocarbon ring having from three to seven carbon atoms. Examples include cyclopropyl, cyclobutyl, cyclopentyl, cyclohexyl and cycloheptyl.

As used herein, the term “N-linked 3- to 7-membered monocyclic heterocyclic ring” refers to a 3, 4, 5, 6 or 7 membered non-aromatic cyclic group containing one to three heteroatom(s) independently selected from N, O and S, which is linked to the rest of the molecule via a nitrogen atom. Examples of N-linked 3- to 7-membered monocyclic heterocyclic rings include aziridinyl, azetidinyl, pyrrolidinyl, imidazolidinyl, pyrazolidinyl, piperidinyl, piperazinyl, morpholinyl, thiomorpholinyl and azepanyl.

As used herein, the term “8- to 11-membered bicyclic heterocyclic ring” refers to a 8, 9, 10 or 11-membered bicyclic group containing one to three heteroatom(s) independently selected from N, O and S, wherein at least one of the rings is non-aromatic. Examples of 8- to 11-membered bicyclic heterocyclic rings in which one of the rings is non-aromatic include dihydrobenzofuranyl, indanyl, indolinyl, isoindolinyl, tetrahydroisoquinolinyl, tetrahydroquinolyl and benzoazepanyl.

As used herein, the term “C₁₋₄alkylene” refers to a straight or branched chain divalent hydrocarbon radical, which contains 1, 2, 3 or 4 carbon atoms. Examples include methylene, ethylene, n-propylene and n-butylene.

As used herein, the term “aryl” refers to phenyl or a 8- to 11-membered bicyclic aromatic group in which at least one of the rings is aromatic. Examples of 8- to 11-membered bicyclic aromatic groups include indenyl, azulenyl, naphthyl and tetrahydronaphthyl.

As used herein, the terms “heteroaryl” and “heteroaromatic group” refer to a 5- or 6-membered monocyclic aromatic group wherein one, two or three carbon atoms are replaced by a heteroatom independently selected from N, O and S, or to a 8- to 11-membered bicyclic aromatic group in which at least one of the rings is aromatic and wherein one to four carbon atoms in total are replaced by a heteroatom independently selected from N, O and S. Examples of 5- or 6-membered monocyclic heteroaromatic groups include furanyl, thienyl, pyrrolyl, oxazolyl, thiazolyl, imidazolyl, oxadiazolyl, thiadiazolyl, pyridyl, triazolyl, triazinyl, pyridazyl, pyrimidinyl, isothiazolyl, isoxazolyl, pyrazinyl, pyrazolyl and pyrimidinyl; examples of 8- to 11-membered bicyclic heteroaromatic groups include quinoxalinyl, quinazolinyl, pyridopyrazinyl, benzoxazolyl, benzothiophenyl, benzimidazolyl, naphthyridinyl, quinolinyl, benzofuranyl, indolyl, benzothiazolyl, oxazolyl[4,5-b]pyridiyl, pyridopyrimidinyl, isoquinolinyl and benzodroxazole.

As used herein, the terms “halogen” and its abbreviation “hal” refer to fluorine, chlorine, bromine, or iodine.

As used herein, the term “salt” refers to any salt of a compound according to the present invention prepared from an inorganic or organic acid or base, quaternary ammonium salts and internally formed salts. Physiologically acceptable salts are particularly suitable for medical applications because of their greater aqueous solubility relative to the parent compounds. Such salts must clearly have a physiologically acceptable anion or cation. Suitably physiologically acceptable salts of the compounds of the present invention include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example benzenesulfonic and p-toluenesulfonic, acids; base addition salts formed with alkali metals and alkaline earth metals and organic bases such as N,N-dibenzylethylenediamine, chloroprocaine, choline, diethanolamine, ethylenediamine, meglumaine (N-methylglucamine), lysine and procaine; and internally formed salts. Salts having a non-physiologically acceptable anion or cation are within the scope of the invention as useful intermediates for the preparation of physiologically acceptable salts and/or for use in non-therapeutic, for example, in vitro, situations.

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include water, ethanol and acetic acid. Most preferably the solvent used is water.

In one embodiment, X is —NR₃R₄; and R₃ and R₄ are independently selected from hydrogen and C₁₋₆alkyl, which C₁₋₆alkyl group is optionally substituted by one or more groups selected from halo, C₁₋₄alkyl, C₁₋₄haloalkyl, C₁₋₄alkoxy, C₁₋₄haloalkoxy, C₁₋₄alkylthio, halo and hydroxy. For example X may be monoC₁₋₆alkylamino or diC₁₋₆alkylamino.

In another embodiment, X is —NR₃R₄; and R₃ and R₄, together with the nitrogen atom to which they are attached, form an N-linked 3- to 7-membered monocyclic heterocyclic ring or an 8- to 11-membered bicyclic heterocyclic ring, which ring optionally comprises one or more further heteroatoms selected from N, O and S; and which ring is optionally substituted by one or more groups selected from halo, C₁₋₄alkyl, C₁₋₄haloalkyl, C₁₋₄alkoxy, C₁₋₄haloalkoxy, C₁₋₄alkylthio, halo and hydroxyl.

Examples of suitable rings include pyrrolidinyl, imidazolidinyl, pyrazolidinyl, oxazolidinyl, isothiazolidinyl, thiazolidinyl, piperidinyl, piperazinyl, morpholinyl, thiomorpholinyl, azetidinyl and azepanyl, each of which is optionally substituted as set out above. For example, the heterocyclic ring formed by R₃ and R₄ is morpholinyl, piperidinyl or azepanyl. The ring may, for example, be optionally substituted by one or more groups selected from halo, C₁₋₄alkyl, C₁₋₄haloalkyl, C₁₋₄alkoxy, C₁₋₄haloalkoxy, C₁₋₄alkylthio, halo and hydroxyl, for example substituted by one to three C₁₋₄alkyl groups.

For example, X is —NR₃R₄ and R₃ and R₄, together with the nitrogen atom to which they are attached, form an N-linked 3-7-membered monocyclic heterocyclic ring, for example a 6-membered monocyclic heterocyclic ring, optionally substituted as set out above, for example substituted with a geminal difluoro group.

In another embodiment, X is —NR₃R₄ and wherein R₃ and R₄, together with the nitrogen to which they are attached, form a 8- to 11-membered bicyclic heterocyclic ring optionally substituted by one or more groups selected from halo, C₁₋₄alkyl, C₁₋₄haloalkyl, C₁₋₄alkoxy, C₁₋₄haloalkoxy, C₁₋₄alkylthio, halo and hydroxyl.

In a further embodiment, X is —NR₃R₄; and R₃ and R₄, together with the nitrogen atom to which they are attached, form an N-linked 3- to 7-membered monocyclic heterocyclic ring or an 8- to 11-membered bicyclic heterocyclic ring, which ring optionally comprises one or more further heteroatoms selected from N, O and S; and which ring is substituted by one or more groups selected from halo, C₁₋₄haloalkyl, C₁₋₄haloalkoxy and C₁₋₄alkylthio. For example the ring may be substituted with one or more halo groups.

In one embodiment, Y is S(O)ₘR₅ where m is 1 or 2 and R₅ is as defined above. For example, Y is S(O)ₘR₅ where m is 2. R₅ is preferably C₁₋₆alkyl, for example methyl.

In one embodiment, n is 0.

In another embodiment, n is 1 or 2 and R₁ is independently hydrogen or C₁₋₄alkyl.

In one embodiment, Z is a phenyl group Z′ as set out above. In an alternatie embodiment, Z is a bicyclic heteraryl group.

In one embodiment, each R₁₃ is independently selected from hydrogen, halogen, cyano and C₁₋₄alkoxyC₁₋₄alkyl.

In one embodiment, each R₁₄ is independently selected from hydrogen, halogen, cyano, nitro, C₁₋₆alkyl, C₁₋₄alkoxy and haloC₁₋₄alkyl.

In one embodiment, each R₁₅ is independently selected from hydrogen, halogen, cyano, amino, nitro, C₁₋₆alkyl, C₁₋₄alkoxy, haloC₁₋₄alkyl, for example trifluoromethyl, C₁₋₄alkylthio, hydroxyC₁₋₄alkyl, C₁₋₄alkoxyC₁₋₄alkyl, C₁₋₄haloalkoxyC₁₋₄alkyl, C₁₋₄alkanoyl, C₁₋₄haloalkanoyl, C₁₋₄alkylsulfonyl, C₁₋₄alkylsulfinyl, C₁₋₄haloalkylsulfinyl, R₉R₁₀NCO(CH₂)ₚ, —CR_{9′}═NR_{10′}, —CR_{9′}═NOR_{10′}, wherein
each R₉ and R₁₀ is independently C₁₋₄alkyl, or where appropriate R₉R₁₀ forms part of a C₃₋₆azacyloalkane or C₃₋₆(2-, 3- or 4-oxo)azacycloalkane ring
each R_{9′} and R_{10′} is independently selected from R₉ and R₁₀ and hydrogen;
p is selected from 0, 1, 2, 3 or 4;

In a further embodiment, Z is a phenyl group Z′ as described herein, and is substituted, and one or more of R₁₃, R₁₄ and R₁₅ is SO₂C₁₋₄alkyl, with the other groups R₁₃, R₁₄ and R₁₅ being selected from hydrogen or halogen for example from hydrogen, chloro or fluoro. In a further embodiment, one or more of R₁₃, R₁₄ and R₁₅ is C₁₋₄alkoxyC₁₋₄alkyl with the other groups R₁₃, R₁₄ and R₁₅ being selected from hydrogen or halo, for example, F or Cl. In a further embodiment, one or more of R₁₃, R₁₄ and R₁₅ is C₁₋₄alkanoyl with the other groups R₁₃, R₁₄ and R₁₅ being selected from hydrogen and halo, for example, F or Cl. In a further embodiment, one or more of R₁₃, R₁₄ and R₁₅ is C₁₋₄alkoxy, for example methoxy, with the other groups R₁₃, R₁₄ and R₁₅ being selected from hydrogen and halo, for example, F or Cl. In a further embodiment, one or more of R₁₃, R₁₄ and R₁₅ is CR₉═C(CN)₂ where R₉ is hydrogen or C₁₋₄alkyl with the other groups R₁₃, R₁₄ and R₁₅ being selected from hydrogen and halo, for example, F or Cl.

In a further embodiment, one or more of R₁₃, R₁₄ and R₁₅ is haloC₁₋₄alkyl, for example trifluoromethyl, with the other groups R₁₃, R₁₄ and R₁₅ being selected from hydrogen and halo, for example F or Cl.

In a further embodiment, one or more of R₁₃, R₁₄ and R₁₅ is selected from C₆₋₁₁arylC₁₋₄alkoxy, C₁₋₄alkylthio, hydroxyC₁₋₄alkyl, C₁₋₄alkoxyC₁₋₄alkyl, C₁₋₄haloalkoxyC₁₋₄alkyl, C₃₋₆cycloalkylC₁₋₄alkyl, C₃₋₆cycloalkyl, C₃₋₆cycloalkylC₁₋₄alkoxy, C₁₋₄alkoxycarbonylC₁₋₄alkyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylamido, C₁₋₄alkylsulfonamidoC₁₋₄alkyl, C₁₋₄alkylamidoC₁₋₄alkyl, C₆₋₁₁arylsulfonamido, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, C₆₋₁₁arylC₁₋₄alkyl, C₁₋₄alkylaminoC₁₋₄alkyl, a group R₉R₁₀NCO(CH₂)ₚ, R_{9′}R_{10′}NSO₂(CH₂)ₚ or R_{9′}SO₂NR_{10′}(CH₂)ₚ, —CR_{9′}═NR_{10′}, —CR_{9′}═NOR_{10′}, —CR_{9′}═C(CN)₂, —CR_{9′}═CH(CN), R_{9′}R_{10′}N(CH₂)_{q}— and R_{9′}R_{10′}N(CH₂)_{q}O—, wherein
each R₉ and R₁₀ is independently C₁₋₄alkyl, or where appropriate R₉R₁₀ forms part of a C₃₋₆azacyloalkane or C₃₋₆(2-, 3- or 4-oxo)azacycloalkane ring
each R_{9′} and R_{10′} is independently selected from R₉ and R₁₀ and hydrogen;
each R_{9″} and R_{10″} is independently selected from R_{9′} and R_{10′} and C₁₋₄alkanoyl;
p is selected from 0, 1, 2, 3 or 4;
q is selected from 2, 3 or 4;

In a further embodiment, one or more of R₁₃, R₁₄ and R₁₅ is selected from hydroxyC₁₋₄alkyl, C₁₋₄alkoxyC₁₋₄alkyl, C₁₋₄haloalkoxyC₁₋₄alkyl, —NR_{9″}R_{10″}, CR_{9′}═NOR_{10′}, and —CR_{9′}═C(CN)₂, wherein
each R_{9′} and R_{10′} is independently hydrogen or C₁₋₄alkyl, and
each R_{9″} and R_{10″} is independently selected from C₁₋₄alkanoyl;

In an alternative embodiment, Z is selected from the group consisting of pyridyl, pyrimidinyl, 1 Hpyrrole[2,3-b]pyridine, pyridazinyl, pyrazinyl, triazolyl, triazinyl, pyrrolyl, imidazolyl, thienyl, furanyl, thiadiazolyl, isoxazolyl, isothiazolyl, thiazolyl, oxadiazolyl and oxazolyl, benzothiazolyl, 1,4-benzodioxinyl, 2,3-dihydro-1,4-benzodioxinyl, benzoxazolyl, indolyl, quinolyl, isoquinolinyl, 1-benzopyranyl, 2-benzopyranyl, dihyrdo-1-benzopyranyl, dihydro-2-benzopyranyl, quinoxalinyl and quinazolinyl, each of which is optionally substituted as set out above. For example, Z may be selected from pyrid-2-yl, pyrimidin-2-yl, pyrimidin-4-yl, quinoxalinyl, quinolinyl, 1H-pyrrolo[2,3-b]pyridinyl. Such groups Z may optionally be substituted with one or more groups selected from amino, cyano, nitro, haloC₁₋₄alkyl, C₁₋₄alkanoyl, hydroxyC₁₋₄alkyl, C₁₋₄alkoxy, C₁₋₄alkoxycarbonyl, C₄₋₉heteroarylsulfonyl, and R_{9″}R_{10″}N—, wherein each R_{9″} and R_{10″} is independently selected from hydrogen, C₁₋₄alkyl and C₁₋₄alkanoyl.

In a further embodiment Z is an optionally substituted pyrrolopyridine or an optionally substituted 1H-pyrrolo[2,3-b]piperidine. Preferred optional substituents are arylsulfonyl and heteroarylsulfonyl, most preferably heteroarylsulfonyl.

Specific examples of compounds of the present invention include compounds of Examples 1 to 63 as out below, and salts and solvates thereof

The compounds of formula (I) may have the ability to crystallise in more than one form. This is a characteristic known as polymorphism, and it is understood that such polymorphic forms (“polymorphs”) are within the scope of formula (I). Polymorphism generally can occur as a response to changes in temperature or pressure or both and can also result from variations in the crystallisation process. Polymorphs can be distinguished by various physical characteristics known in the art such as x-ray diffraction patterns, solubility, and melting point.

Certain of the compounds described herein may exist in stereoisomeric forms (i.e. they may contain one or more asymmetric carbon atoms or may exhibit cis-trans isomerism). The individual stereoisomers (enantiomers and diastereoisomers) and mixtures of these are included within the scope of the present invention. Likewise, it is understood that compounds of formula (I) may exist in tautomeric forms other than that shown in the formula and these are also included within the scope of the present invention.

As referred to above, individual enantiomers of compounds of formula (I) may be prepared and an indication of the preferred stereochemistry for such enantiomers has been given. In a preferred embodiment, an optically pure enantiomer is desired. The term “optically pure enantiomer” means that the compound contains greater than about 90% of the desired isomer by weight, preferably greater than about 95% of the desired isomer by weight, and most preferably greater than about 99% of the desired isomer by weight, said weight percent based upon the total weight of the isomer(s) of the compound.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the working Examples.

Compounds of general formula (I) may be prepared by methods disclosed in the documents hereinbefore referred to and by methods known in the art of organic synthesis as set forth in part by the following synthesis schemes. It is also recognised that in all of the schemes described below, it is well understood that protecting groups for sensitive or reactive groups are employed where necessary in accordance with general principles of chemistry. Protecting groups are manipulated according to standard methods of organic synthesis (T. W. Greene and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I). Those skilled in the art will recognise if a stereocentre exists in compounds of formula (I). Accordingly, the present invention includes both possible stereoisomers and includes not only racemic compounds but the individual enantiomers as well. Where the stereochemistry is indicated as being variable at certain positions, a mixture of stereoisomers may be obtained, this mixture having been separated where indicated. Stereoisomers may be separated by high-performance liquid chromatography or other appropriate means. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

Typical reaction routes for the preparation of a compound of formula (I) as hereinbefore defined, are shown in the following schemes. It should be noted that, while the schemes illustrate cases wherein Y is —SO₂Me and n is zero, the schemes are applicable for other cases wherein n and Y are as defined for formula (I) above. Similarly, the schemes illustrate cases where the leaving group is chlorine, but the leaving group may be any other suitable group. Scheme 5 illustrates methodology for preparing compounds in which Z is phenyl substituted with an alkoxyalkyl group. The methodology is also suitable for the preparation of other molecules of the invention that comprise alkoxyalkyl groups. Starting materials and reagents are known to the skilled person in the art and/or can be prepared using methods known in the art.

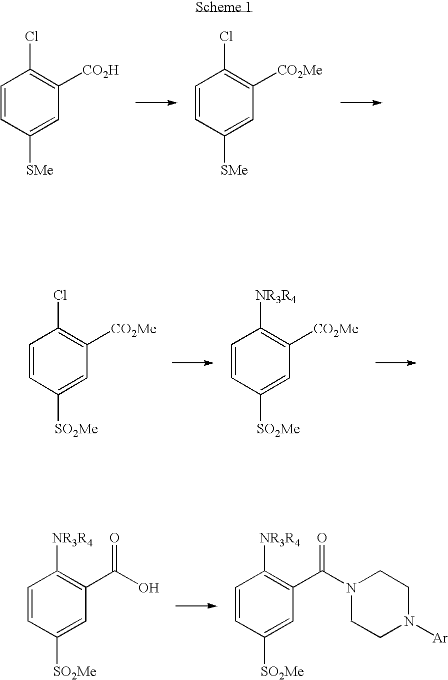

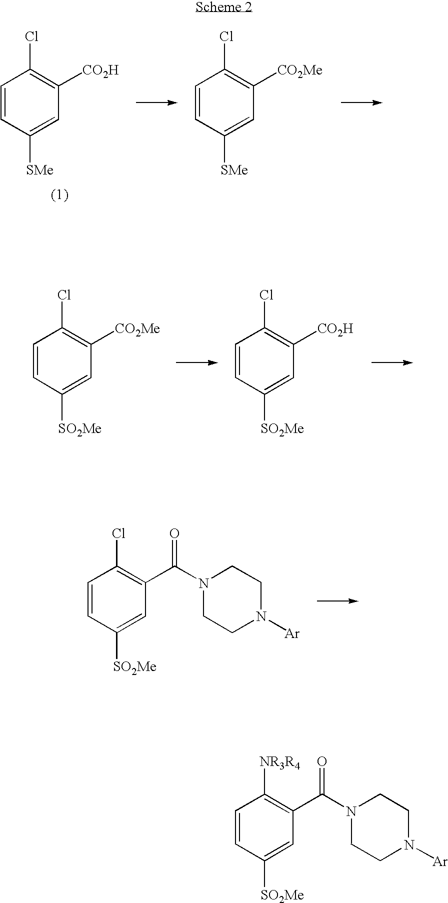

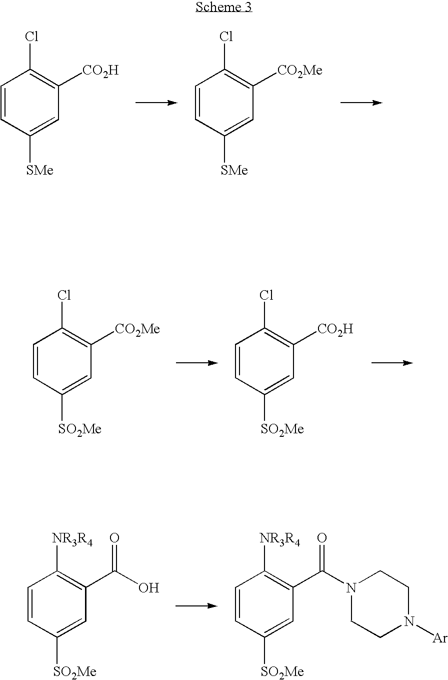

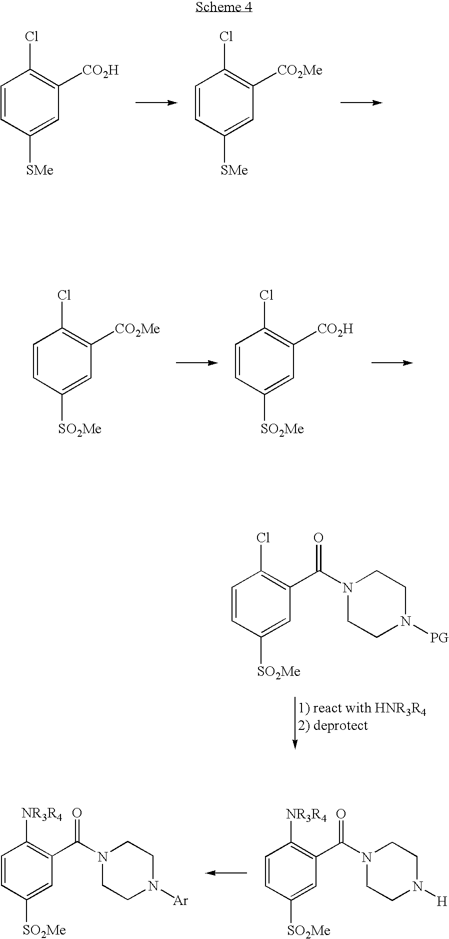

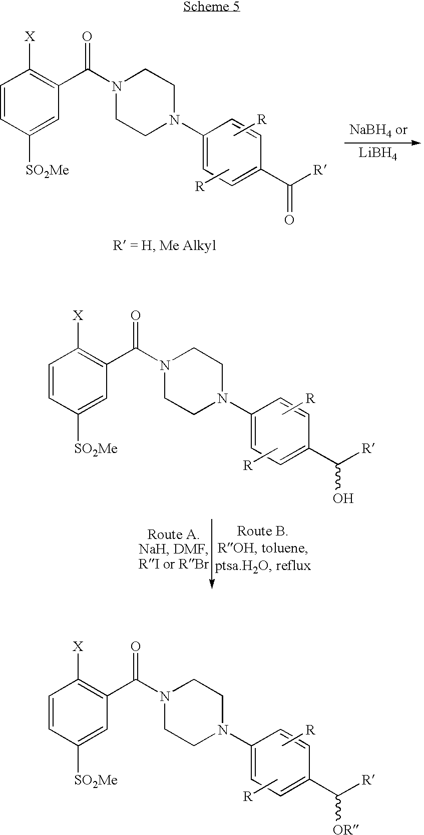

Accordingly, in a second aspect, the present invention provides a method of preparing a compound of formula (I), comprising the step of:
(a) reacting a compound of formula (II): 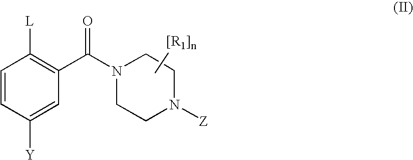
wherein L is a leaving group such as halogen or triflate, and Y, R₁, n and Z are as defined for formula (I), with a compound of formula (III):
H—X (III)
wherein X is as defined for formula (I) and H is hydrogen; or
(b) reacting a compound of formula (IV): 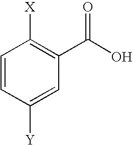
wherein X and Y are as defined for formula (I), with a compound of formula (V): 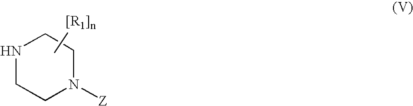
wherein R₁, n and Z are as defined for formula (I); or
(c) reacting a compound of formula (VI): 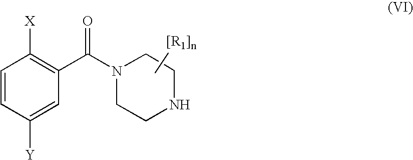
wherein X, Y, n and R₁ are as defined for formula (I), with a group Z-L wherein Z is as defined for formula (I) and L is a leaving group such as halogen or triflate, under basic conditions with a suitable catalyst such as palladium acetate, and a suitable ligand such as 2,2′-bis(diphenylphosphino)-1,1-binaphthyl; or by heating a compound of formula (VI) with a group Z-L to 180° C., with or without diisopropylamine as solvent, in a microwave reactor;
and thereafter optionally for step (a), step (b) or step (c),
removing any protecting groups and/or
converting a compound of formula (I) into another compound of formula (I) and/or
forming a salt or solvate.

Step (c) may be carried out under suitable reaction conditions known in the literature, for example in J P Wolfe, H Tomori, J P Sadighi, J Yin and S L Buchwald, J. Org. Chem. (2000), 65, 1158; Org. Lett. (2003), 5(14), 2413; or J P Wolfe and S L Buchwald, J. Org. Chem. (2000), 65, 1144.

Compounds of formulae (If)-(VI) are commercially available, or may be made according to known methods available to the skilled person, or may be made according to methods disclosed herein.

Compounds of formula (V) (piperazines) are either commercially available or may be prepared by following literature methods:—
1. D. V. Gardner and A. C. Goudie, Ger. Offen. (1978), DE 2753878 (Becham Group)
2. Hakan V. Wikström, Marguérite M. Mensonides-Harsema, Thomas I. F. H. Cremers, Ejner K. Moltzen, and Jørn Arnt J Med Chem 2002, 45, 3280-3285
3. Harsha G. Jaisinghani and Bhushan M. Khadilkar Tet Lett 1997, 38, 6875-6876
4. C. Yamato, T. Takahishi, T. Fujita, S. Kuriyama, N. Hirose Xenobiotica 1974, 4, 765-777.

Compounds of formula (I) can be converted into further compounds of formula (I) using standard techniques. For example, and by way of illustration rather than limitation, possible conversion reactions include acylation with an appropriate acylating agent such as acetyl chloride, alkylation using an appropriate alkylating reagent such as methyl iodide, and sulfonylation using a sulfonylating agent such as methanesulfonic anhydride.

Pharmaceutically acceptable salts may be prepared conventionally by reaction with the appropriate acid or acid derivative.

The compounds of the present invention inhibit the GlyT1 transporter. The compounds may selectively inhibit the GlyT1 transporter over the GlyT2 transporter.

Such compounds would be suitable for the treatment of certain neurological and neuropsychiatric disorders. As used herein, the terms “treatment” and “treating” refer to the alleviation and/or cure of established symptoms as well as prophylaxis.

The affinities of the compounds of this invention for the GlyT1 transporter can be determined by the following assay:

HEK293 cells expressing the Glycine (Type 1) transporter were grown in cell medium (DMEM/NUT mix F12) containing 2 mM L-Glutamine, 0.8 mg/mL G418 and 10% heat inactivated fetal calf serum (Gibco BRL) at 37° C. in 5% CO₂. Cells grown to 70-80% confluency in T175 flasks were harvested and resuspended at 1.6×10⁶ cells/ml in assay buffer [NaCl (140 mM), KCl (5.4 mM), CaCl₂ (1.8 mM), MgSO₄ (0.8 mM), HEPES (20 mM), glucose (5 mM) and alanine (5 mM), pH 7.4]. An equal volume of Leadseeker™ SPA beads (12.5 mg/ml suspended in assay buffer) was added to the cells and 25 mL of the cell/bead suspension transferred to each well of a 384-well white solid bottom plate (20,000 cells/well) that contained 14 mL of assay buffer. Compounds were prepared as 10 mM stocks in DMSO. Two-fold serial dilutions of the compounds were made in DMSO from a top concentration of 5 mM. 1 mL of compound at each concentration was added to the assay plate using 384-well parallel dispensing. Substrate (10 mL) was added to each well [1:40 dilution of [³H]-glycine in assay buffer containing 5 mM glycine). Final DMSO concentration=2%. Data was collected using a PerkinElmer Viewlux as 5 minute exposures. IC₅₀ values were determined using Grafit.

The following alternative assay may also be used:

HEK293 cells expressing the Glycine (Type 1) transporter were grown in cell medium (DMEM/NUT mix F12) containing 2 mM L-Glutamine, 0.8 mg/mL G418 and 10% heat inactivated fetal calf serum (Gibco BRL) at 37° C. in 5% CO₂. Cells grown to 70-80% confluency in T175 flasks were harvested and resuspended at 4×10⁵ cells/ml in assay buffer [NaCl (140 mM), KCl (5.4 mM), CaCl₂ (1.8 mM), MgSO₄ (0.8 mM), HEPES (20 mM), glucose (5 mM) and alanine (5 mM), pH 7.4]. An equal volume of Leadseeker SPA beads (12.5 mg/ml suspended in assay buffer) was added to the cell suspension. Compounds were prepared as 10 mM stocks in DMSO. 2.5 fold serial dilutions of the compounds were made in DMSO from a top conc of 2.5 mM. 100 mL of compound at each concentration was added to the assay plate (384-well white solid bottom plate) using the hummingbird dispenser. 5 uL of the cell/bead mix was then added on top of the compound using a multidrop dispenser. Substrate (5 uL) was then added to each well (1:100 dilution of H3-glycine in assay buffer containing 2.5 uM glycine) Data was collected using a PerkinElmer Viewlux as 5 minute exposures. plC50 data values were determined using Activity Base.

Compounds are considered to have activity at the GlyT1 transporter if they have a pIC₅₀ of 5.0 or above. The example compounds below were found to have a pIC₅₀ at the GlyT1 transporter of greater than 5.0. Preferred compounds of the invention were found to have a pIC₅₀ at the GlyT1 transporter of greater than 6.0.

Compounds for use according to the invention may be administered as the raw material but the active ingredients are preferably provided in the form of pharmaceutical compositions.

Accordingly, in a further aspect of the invention, there is provided a pharmaceutical composition comprising a compound of formula (I) as hereinbefore described or a salt or solvate thereof, and at least one pharmaceutically acceptable carrier, diluent or excipient. These pharmaceutical compositions may be used in the treatment of clinical conditions for which a GlyT1 inhibitor is indicated such as, for example, schizophrenia. The carrier must be pharmaceutically acceptable to the recipient and must be compatible with, i.e. not have a deleterious effect upon, the other ingredients in the composition. The carrier may be a solid or a liquid and is preferably formulated with at least one compound of formula (I) or a salt or solvate thereof as a unit dose formulation. If desired, other physiologically active ingredients may also be incorporated in the pharmaceutical compositions of the invention.

These pharmaceutical compositions may be used in the treatment of clinical conditions for which a GlyT1 inhibitor is indicated such as, for example, schizophrenia. The carrier must be pharmaceutically acceptable to the recipient and must be compatible with, i.e. not have a deleterious effect upon, the other ingredients in the composition. The carrier may be a solid or a liquid and is preferably formulated with at least one compound of formula (I) or a salt or solvate thereof as a unit dose formulation. If desired, other physiologically active ingredients may also be incorporated in the pharmaceutical compositions of the invention.

Within the context of the present invention, the terms used herein are classified in the Diagnostic and Statistical Manual of Mental Disorders, 4^{th} Edition, published by the American Psychiatric Association (DSM-IV) and/or the International Classification of Diseases, 10^{th} Edition (ICD-10). The various subtypes of the disorders mentioned herein are contemplated as part of the present invention. Numbers in brackets after the listed diseases below refer to the classification code in DSM-IV.

In particular, the compounds of formula (I) are of use in the treatment of schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

The compounds of formula (I) are also of use in the treatment of mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

The compounds of formula (I) are also of use in the treatment of anxiety disorders including Panic Attack, Agoraphobia, Panic Disorder, Agoraphobia Without History of Panic Disorder (300.22), Specific Phobia (300.29) including the subtypes Animal Type, Natural Environment Type, Blood-Injection-Injury Type, Situational Type and Other Type), Social Phobia (300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder and Anxiety Disorder Not Otherwise Specified (300.00).

The compounds of formula (I) are also of use in the treatment of substance-related disorders including Substance Use Disorders such as Substance Dependence and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-induced Mood Disorder, Alcohol-Induced Anxiety Disorder, Alcohol-induced Sexual Dysfunction, Alcohol-induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-induced Anxiety Disorder, Amphetamine-Induced Sexual Dysfunction, Amphetamine-induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-induced Psychotic Disorder, Hallucinogen-induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-induced Psychotic Disorder, Inhalant-induced Mood Disorder, Inhalant-induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

The compounds of formula (I) are also of use in the treatment of sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type.

The compounds of formula (I) are also of use in the treatment of eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; and Eating Disorder Not Otherwise Specified (307.50).

The compounds of formula (I) are also of use in the treatment of Autistic Disorder (299.00); Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset-Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

The compounds of formula (I) are also of use in the treatment of Personality Disorders including the subtypes Paranoid Personality Disorder (301.0), Schizoid Personality Disorder (301.20), Schizotypal Personality Disorder (301.22), Antisocial Personality Disorder (301.7), Borderline Personality Disorder (301.83), Histrionic Personality Disorder (301.50), Narcissistic Personality Disorder (301.81), Avoidant Personality Disorder (301.82), Dependent Personality Disorder (301.6), Obsessive-Compulsive Personality Disorder (301.4) and Personality Disorder Not Otherwise Specified (301.9).

The compounds of Formula (I) are also of use in the enhancement of cognition including the treatment of cognition impairment in other diseases such as schizophrenia, bipolar disorder, depression, other psychiatric disorders and psychotic conditions associated with cognitive impairment. Within the context of the present invention, the term cognitive impairment includes for example the treatment of impairment of cognitive functions including attention, orientation, learning disorders, memory (i.e. memory disorders, amnesia, amnesic disorders, transient global amnesia syndrome and age-associated memory impairment) and language function; cognitive impairment as a result of stroke, Alzheimer's disease, Huntington's disease, Pick disease, Aids-related dementia or other dementia states such as Multiinfarct dementia, alcoholic dementia, hypotiroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, mild cognitive impairment, age related cognitive impairment, autism related cognitive impairment, Down's syndrome, cognitive deficit related to psychosis, and post-electroconvulsive treatment related cognitive disorders; and dyskinetic disorders such as Parkinson's disease, neuroleptic-induced parkinsonism, and tardive dyskinesias.

The compounds of formula (I) are also of use in the treatment of sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

The invention also provides a compound of formula (I) as hereinbefore described or a pharmaceutically acceptable salt or solvate thereof for use in the treatment of schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders.

The invention also provides a compound of formula (I) as hereinbefore described or a pharmaceutically acceptable salt or solvate thereof for use in the treatment of psychotic disorders, schizophrenia, Parkinson's disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

In another aspect of the invention, there is provided a method of treating a mammal, including a human, suffering from or susceptible to a disorder mediated by GlyT1, which comprises administering an effective amount of a compound of formula (I) as hereinbefore defined or a salt or solvate thereof.

The invention also provides a method of treating schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders which comprises administering to a mammal in need thereof an effective amount of a compound of formula (I) as hereinbefore described or a pharmaceutically acceptable salt or solvate thereof.

The invention also provides a method of treating psychotic disorders, schizophrenia, Parkinson's disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders which comprises administering to a mammal in need thereof an effective amount of a compound of formula (I) as hereinbefore described or a pharmaceutically acceptable salt or solvate thereof.

In another aspect of the invention, there is provided use of a compound of formula (I) as hereinbefore defined or a salt or solvate thereof in the preparation of a medicament for the treatment of a disorder mediated by GlyT1.

Preferably, the disorder mediated by GlyT1 to be treated by the use or method as hereinbefore described is a psychosis, including schizophrenia, dementia and attention deficit disorders, particularly schizophrenia.

The invention also provides the use of a compound of formula (I) as hereinbefore described or a pharmaceutically acceptable salt or solvate thereof in the manufacture of a medicament for the treatment of schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, Parkinson's disease, dyskinetic disorders, depression, bipolar disorder, cognitive impairment, obesity, emesis, movement disorders, obsessive-compulsive disorders, amnesia, aggression, vertigo, dementia and circadian rhythm disorders.

The invention also provides the use of a compound of formula (I) as hereinbefore described or a pharmaceutically acceptable salt or solvate thereof in the manufacture of a medicament for the treatment of psychotic disorders, schizophrenia, Parkinson's disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

As used herein, the term “effective amount” means that amount of a drug or pharmaceutical agent that will elicit the biological or medical response of a tissue, system, animal or human that is being sought, for instance, by a researcher or clinician.

Compounds for use according to the invention may be administered as the raw material but the active ingredients are preferably provided in the form of pharmaceutical compositions.

Accordingly, in a further aspect of the invention, there is provided a pharmaceutical composition comprising a compound of formula (I) as hereinbefore described or a salt or solvate thereof, and at least one pharmaceutically acceptable carrier, diluent or excipient.

These pharmaceutical compositions may be used in the treatment of clinical conditions for which a GlyT1 inhibitor is indicated such as, for example, schizophrenia. The carrier must be pharmaceutically acceptable to the recipient and must be compatible with, i.e. not have a deleterious effect upon, the other ingredients in the composition. The carrier may be a solid or a liquid and is preferably formulated with at least one compound of formula (I) or a salt or solvate thereof as a unit dose formulation. If desired, other physiologically active ingredients may also be incorporated in the pharmaceutical compositions of the invention. It will be appreciated by those skilled in the art that the compounds according to the invention may advantageously be used in conjunction with one or more other therapeutic agents, for instance, different antidepressant agents such as 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists and/or anticonvulsant agents, as well as atypical antipsychotic drugs and cognitive enhancers.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Suitable anticonvulsant agents which may be used in combination of the compounds of the invention include for example divalproex, carbamazepine and diazepam.

Suitable atypical antipsychotic drugs which may be used in combination of the compounds of the invention include for example risperidone, olanzapine, ziprasidone, aripiprazole and clozapine.

It will be appreciated that the compounds of the combination or composition may be administered simultaneously (either in the same or different pharmaceutical formulations), separately or sequentially.

The compounds of formula (I) and their pharmaceutically acceptable salts and solvates thereof are also suitable for combination with other typical and atypical antipsychotics to provide improved treatment of psychotic disorders. Particular advantages associated with the combinations, uses and methods of treatment of compounds of formula (I) and their pharmaceutically acceptable salts and solvates thereof include equivalent or improved efficacy at doses of administration which are lower than those commonly used for the individual components. Improved treatments of positive symptoms and/or negative symptoms and/or cognitive symptoms of the psychotic disorder may also be observed. The combinations, uses and methods of treatment of the invention may also provide advantages in treatment of patients who fail to respond adequately or who are resistant to treatment with certain neuroleptic agents.

The combination therapies of the invention are preferably administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of the compounds of formula (I) or a pharmaceutically acceptable salt or solvate thereof and at least one neuroleptic agent are within the scope of the current invention. In one embodiment of adjunctive therapeutic administration as described herein, a patient is typically stabilised on a therapeutic administration of one or more of the of the components for a period of time and then receives administration of another component. Within the scope of this invention, it is preferred that the compounds of formula (I) or a pharmaceutically acceptable salt or solvate thereof is administered as adjunctive therapeutic treatment to patients who are receiving administration of at least one neuroleptic agent, but the scope of the invention also includes the adjunctive therapeutic administration of at least one neuroleptic agent to patients who are receiving administration of compounds of formula (I) or a pharmaceutically acceptable salt or solvate thereof.

The combination therapies of the invention may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts.

In a further aspect therefore, the invention provides a method of treatment of a psychotic disorder by adjunctive therapeutic administration of compounds of formula (I) or a pharmaceutically acceptable salt or solvate thereof to a patient receiving therapeutic administration of at least one neuroleptic agent. In a further aspect, the invention provides the use of compounds of formula (I) or a pharmaceutically acceptable salt or solvate thereof in the manufacture of a medicament for adjunctive therapeutic administration for the treatment of a psychotic disorder in a patient receiving therapeutic administration of at least one neuroleptic agent. The invention further provides compounds of formula (I) or a pharmaceutically acceptable salt or solvate thereof for use for adjunctive therapeutic administration for the treatment of a psychotic disorder in a patient receiving therapeutic administration of at least one neuroleptic agent.

In a further aspect, the invention provides a method of treatment of a psychotic disorder by adjunctive therapeutic administration of at least one neuroleptic agent to a patient receiving therapeutic administration of compounds of formula (I) or a pharmaceutically acceptable salt or solvate thereof. In a further aspect, the invention provides the use of at least one neuroleptic agent in the manufacture of a medicament for adjunctive therapeutic administration for the treatment of a psychotic disorder in a patient receiving therapeutic administration of compounds of formula (I) or a pharmaceutically acceptable salt or solvate thereof. The invention further provides at least one neuroleptic agent for adjunctive therapeutic administration for the treatment of a psychotic disorder in a patient receiving therapeutic administration of compounds of formula (I) or a pharmaceutically acceptable salt or solvate thereof.

In a further aspect, the invention provides a method of treatment of a psychotic disorder by simultaneous therapeutic administration of compounds of formula (I) or a pharmaceutically acceptable salt or solvate thereof in combination with at least one neuroleptic agent. The invention further provides the use of a combination of compounds of formula (I) or a pharmaceutically acceptable salt or solvate thereof and at least one neuroleptic agent in the manufacture of a medicament for simultaneous therapeutic administration in the treatment of a psychotic disorder. The invention further provides the use of compounds of formula (I) or a pharmaceutically acceptable salt thereof in the manufacture of a medicament for simultaneous therapeutic administration with at least one neuroleptic agent in the treatment of a psychotic disorder. The invention further provides compounds of formula (I) or a pharmaceutically acceptable salt thereof for use for simultaneous therapeutic administration with at least one neuroleptic agent in the treatment of a psychotic disorder. The invention further provides the use of at least one neuroleptic agent in the manufacture of a medicament for simultaneous therapeutic administration with compounds of formula (I) or a pharmaceutically acceptable salt thereof in the treatment of a psychotic disorder.

In further aspects, the invention provides a method of treatment of a psychotic disorder by simultaneous therapeutic administration of a pharmaceutical composition comprising compounds of formula (I) or a pharmaceutically acceptable salt or solvate thereof and at least one mood stabilising or antimanic agent, a pharmaceutical composition comprising compounds of formula (I) or a pharmaceutically acceptable salt or solvate thereof and at least one mood stabilising or antimanic agent, the use of a pharmaceutical composition comprising compounds of formula (I) or a pharmaceutically acceptable salt or solvate thereof and at least one mood stabilising or antimanic agent in the manufacture of a medicament for the treatment of a psychotic disorder, and a pharmaceutical composition comprising compounds of formula (I) or a pharmaceutically acceptable salt or solvate thereof and at least one mood stabilising or antimanic agent for use in the treatment of a psychotic disorder.

In a further aspect, the invention provides a kit-of-parts for use in the treatment of a psychotic disorder comprising a first dosage form comprising compounds of formula (I) or a pharmaceutically acceptable salt or solvate thereof and one or more further dosage forms each comprising a neuroleptic agent for simultaneous therapeutic administration.

Within the context of the present invention, the term psychotic disorder includes those disorders mentioned above, such as schizophrenia, mood disorders, anxiety disorders, substance-related disorders, sleep disorders, eating disorders, autistic disorder, attention-deficit/hyperactivity disorder, disruptive behaviour disorder, tic disorders, personality disorders, cognition impairment in other diseases, sexual dysfunction, dyskinetic disorders, depression, bipolar disorder, cognitive impairment and obsessive-compulsive disorders and all the various forms of the disorders as mentioned herein. which are contemplated as part of the present invention.

Examples of neuroleptic/antipsychotic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene thienobenzodiazepines; dibenzodiazepines; benzisoxazoles; dibenzothiazepines; imidazolidinones; benzisothiazolyl-piperazines; triazine such as lamotrigine; dibenzoxazepines, such as loxapine; dihydroindolones, such as molindone; aripiprazole; and derivatives thereof that have antipsychotic activity.

Examples of neuroleptic drugs that are preferred for use in the present invention are shown in Table 1.

TABLE 1
Neuroleptic drugs

| | | | | Dosage |
|---|---|---|---|---|
| Common | | Route of | | Range and |
| Name | Trade Name | Administration | Form | (Median)^{a} |
| Clozapine | CLOZARIL | oral | tablets | 12.5-900 mg/day |
| | | | | (300-900 mg/day) |
| Olanzapine | ZYPREXA | oral | tablets | 5-25 mg/day |
| | | | | (10-25 mg/day) |
| Ziprasidone | GEODON | oral | capsules | 20-80 mg/twice a day |
| | | | | (80-160 mg/day) |
| Risperidone | RISPERDAL | oral | solution tablets | 2-16 mg/day tablets |
| | | | | (4-12 mg/day) |
| Quetiapine | SEROQUEL | oral | tablets | 50-900 mg/day |
| fumarate | | | | (300-900 mg/day) |
| Sertindole | SERLECT | | | (4-24 mg/day) |
| Amisulpride | | | | |
| Haloperidol | HALDOL | oral | tablets | 1-100 mg/day |
| | | | | (1-15 mg/day) |
| Haloperidol | HALDOL | parenteral | injection | |
| Decanoate | Decanoate | | | |
| Haloperidol lactate | HALDOL | oral | solution | |
| | INTENSOL | parenteral | injection | |
| Chlorpromazine | THORAZINE | rectal | suppositories | 30-800 mg/day |
| | | oral | capsules | (200-500 mg/day) |
| | | | solution | |
| | | | tablets | |
| | | parenteral | injection | |
| Fluphenazine | PROLIXIN | | | 0.5-40 mg/day |
| | | | | (1-5 mg/day) |
| Fluphenazine | PROLIXIN | parenteral | injection | (about one-half |
| decanoate | Decanoate | | | the dosage shown |
| | | | | for oral) |
| Fluphenazine | PROLIXIN | parenteral | injection | (same as above |
| enanthate | | | | |
| Fluphenazine | PROLIXIN | oral | elixer | |
| hydrochloride | | | solution | |
| | | parenteral | injection | |
| Thiothixene | NAVANE | oral | capsules | 6-60 mg/day |
| | | | | (8-30 mg/day) |
| Thiothixene | NAVANE | oral | solution | |
| hydrochloride | | parenteral | injection | |
| Trifluoperazine | STELAZINE | | | (2-40 mg/day) |
| Perphenazine | TRILAFON | oral | solution | 12-64 mg/day |
| | | | tablets | (16-64 mg/day) |
| | | parenteral | injection | |
| Perpehazine and | ETRAFON | oral | tablets | |
| Amitriptyline | TRIAVIL | | | |
| hydrochloride | | | | |
| Thioridazine | MELLARIL | oral | suspension | 150-800 mg/day |
| | | | solution | (100-300 mg/day) |
| | | | tablets | |
| Mesoridazine | | | | (30-400 mg/day) |
| Molindone | MOBAN | | | 50-225 mg/day |
| | | | | (15-150 mg/day) |
| Molindone | MOBAN | oral | solution | |
| hydrochloride | | | | |
| Loxapine | LOXITANE | | | 20-250 mg/day |
| | | | | (60-100 mg/day) |
| Loxapine | LOXITANE | oral | solution | |
| hydrochloride | | parenteral | injection | |
| Loxapine | LOXITANE | oral | capsules | |
| succinate | | | | |
| Pimozide | | | | (1-10 mg/day) |
| Flupenthixol | | | | |
| Promazine | SPARINE | | | |
| Triflupromazine | VESPRIN | | | |
| Chlorprothixene | TARACTAN | | | |
| Droperidol | INAPSINE | | | |
| Acetophenazine | TINDAL | | | |
| Prochlorperazine | COMPAZINE | | | |
| Methotrimeprazine | NOZINAN | | | |
| Pipotiazine | PIPOTRIL | | | |
| Aripiprazole | | | | |
| Hoperidone | | | | |

Examples of tradenames and suppliers of selected neuroleptic drugs are as follows clozapine (available under the tradename CLOZARIL®, from Mylan, Zenith Goldline, UDL, Novartis); olanzapine (available under the tradename ZYPREX®, from Lilly ziprasidone (available under the tradename GEODON®, from Pfizer); risperidone (available under the tradename RISPERDAL®, from Janssen); quetiapine fumarate (available under the tradename SEROQUEL®, from AstraZeneca); haloperidol (available under the tradename HALDOL®, from Ortho-McNeil); chlorpromazine (available under the tradename THORAZINE®, from SmithKline Beecham (GSK); fluphenazine (available under the tradename PROLIXIN®, from Apothecon, Copley, Schering, Teva, and American Pharmaceutical Partners, Pasadena); thiothixene (available under the tradename NAVANE®; from Pfizer); trifluoperazine (10-[3-(4-methyl-1-piperazinyl)propyl]-2-(trifluoromethyl)phenothiazine dihydrochloride, available under the tradename STELAZINE®, from Smith Klein Beckman; perphenazine (available under the tradename TRILAFON®; from Schering); thioridazine (available under the tradename MELLARIL®; from Novartis, Roxane, HiTech, Teva, and Alpharma); molindone (available under the tradename MOBAN®, from Endo); and loxapine (available under the tradename LOXITANE®; from Watson). Furthermore, benperidol (Glianimon®), perazine (Taxilan®) or melperone (Eunerpan®)) may be used.

Other preferred neuroleptic drugs include promazine (available under the tradename SPARINE®), triflurpromazine (available under the tradename VESPRIN®), chlorprothixene (available under the tradename TARACTAN®), droperidol (available under the tradename INAPSINE®), acetophenazine (available under the tradename TINDAL®;), prochlorperazine (available under the tradename COMPAZINE®), methotrimeprazine (available under the tradename NOZINAN®), pipotiazine (available under the tradename PIPOTRIL®), ziprasidone, and hoperidone.

Particularly preferred neuroleptic agents for use in the invention are olanzapine, risperidone, quetiapine, aripiprazole, haloperidol, clozapine, ziprasidone and osanetant.

It will be appreciated by those skilled in the art that the compounds according to the invention may advantageously be used in conjunction with one or more other therapeutic agents, for instance, different antidepressant agents such as 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists and/or anticonvulsant agents, as well as atypical antipsychotic drugs and cognitive enhancers.

Suitable 5HT3 antagonists which may be used in combination of the compounds of the inventions include for example ondansetron, granisetron, metoclopramide. Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Suitable anticonvulsant agents which may be used in combination of the compounds of the invention include for example divalproex, carbamazepine and diazepam.

Suitable atypical antipsychotic drugs which may be used in combination of the compounds of the invention include for example risperidone, olanzapine, ziprasidone, aripiprazole and clozapine.

It will be appreciated that the compounds of the combination or composition may be administered simultaneously (either in the same or different pharmaceutical formulations), separately or sequentially.

In a further aspect of the invention, there is provided a compound of formula (I) as hereinbefore described or a salt or solvate thereof, for use in therapy.

In another aspect of the invention, there is provided a compound of formula (I) as hereinbefore described or a salt or solvate thereof, for use in the treatment of a disorder mediated by GlyT1.

As used herein, the term “a disorder mediated by GlyT1” refers to a disorder that may be treated by the administration of a medicament that alters the activity of the GlyT1 transporter. As hereinbefore described, the action of GlyT1 transporters affects the local concentration of glycine around NMDA receptors. As a certain amount of glycine is needed for the efficient functioning of NMDA receptors, any change to that local concentration can affect NMDA-mediated neurotransmission. As hereinbefore described, changes in NMDA-mediated neurotransmission have been implicated in certain neuropsychiatric disorders such as dementia, depression and psychoses, for example schizophrenia, and learning and memory disorders, for example attention deficit disorders and autism. Thus, alterations in the activity of the GlyT1 transporter are expected to influence such disorders.

The disorders mediated by GlyT1 referred to herein include neurological and neuropsychiatric disorders, including psychoses such as schizophrenia, dementia and other forms of impaired cognition such as attention deficit disorders and organic brain syndromes. Other neuropsychiatric disorders include drug-induced (phencyclidine, ketamine and other dissociative anesthetics, amphetamine and other psychostimulants and cocaine) psychosis, psychosis associated with affective disorders, brief reactive psychosis, schizoaffective psychosis, and psychosis NOS, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), and NMDA receptor-related disorders such as autism, depression, benign forgetfulness, childhood learning disorders and closed head injury.

In a further aspect of the invention, there is provided a method of treating a mammal, including a human, suffering from or susceptible to a disorder mediated by GlyT1, which comprises administering an effective amount of a compound of formula (Ib) or a salt or solvate thereof: 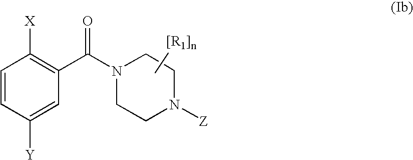
wherein
X is —NR₃R₄, wherein
   R₃ and R₄ are independently selected from hydrogen and C₁₋₆alkyl, or R₃ and R₄, together with the nitrogen atom to which they are attached, form an N-linked 3- to 7-membered monocyclic heterocyclic ring or an 8- to 11-membered bicyclic heterocyclic ring, which ring optionally comprises one or more further heteroatoms selected from N, O and S; and which C₁₋₆alkyl group or ring is optionally substituted by one or more groups selected from halo, C₁₋₄alkyl, C₁₋₄haloalkyl, C₁₋₄alkoxy, C₁₋₄haloalkoxy, C₁₋₄alkylthio, halo and hydroxy;
Y is S(O)ₘR₅ or —SO₂NR₆R₇ wherein
   m is 1 or 2; and
   R₅ is selected from C₁₋₆alkyl, C₃₋₇cycloalkyl, C₅₋₁₁aryl and C₄₋₁₀heteroaryl, which C₁₋₆alkyl, C₃₋₇cycloalkyl, C₅₋₁₁aryl or C₄₋₁₀heteroaryl is optionally substituted with one or two groups selected from halo, C₁₋₄alkoxy and C₁₋₄haloalkoxy;
   R₆ and R₇ are independently selected from hydrogen and C₁₋₆alkyl but are not both simultaneously C₁₋₆alkyl; which C₁₋₆alkyl is optionally substituted with one or more groups selected from halo, C₁₋₄alkoxy and C₁₋₄haloalkoxy;
n is 0, 1 or 2,
each R₁ is independently selected from C₁₋₆alkyl, halo, C₁₋₆haloalkyl C₁₋₄alkoxy and C₁₋₄haloalkoxy;
Z is an optionally substituted phenyl group Z′: 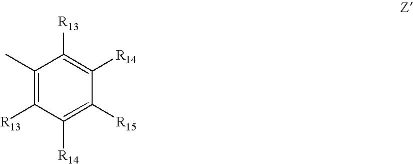
   wherein each R₁₃ is independently selected from hydrogen, halogen, hydroxy, cyano, amino, nitro, C₁₋₆alkyl, haloC₁₋₄alkyl, haloC₁₋₄alkoxy, C₆₋₁₁arylC₁₋₄alkoxy, C₁₋₄alkylthio, hydroxyC₁₋₄alkyl, C₁₋₄alkoxyC₁₋₄alkyl, C₃₋₆cycloalkylC₁₋₄alkyl, C₃₋₆cycloalkyl, C₃₋₆cycloalkylC₁₋₄alkoxy, C₁₋₄alkanoyl, C₁₋₄alkoxycarbonyl, C₁₋₄alkoxycarbonylC₁₋₄alkyl, C₁₋₄alkylsulfonyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylamido, C₁₋₄alkylsulfonamidoC₁₋₄alkyl, C₁₋₄alkylamidoC₁₋₄alkyl, C₆₋₁₁arylsulfonamido, C₆₋₁₁arylcarboxamido, C₆₋₁₁arylsulfonamidoC₁₋₄alkyl, C₆₋₁₁ arylcarboxamidoC₁₋₄alkyl, C₆₋₁₁aroyl, C₆₋₁₁aroylC₁₋₄alkyl, C₆₋₁₁arylC₁₋₄alkanoyl, C₁₋₄acyl, C₆₋₁₁arylC₁₋₄alkyl, C₁₋₄alkylaminoC₁₋₄alkyl, a group R_{9″}R_{10″}N—, R₉R₁₀NCO(CH₂)ₚ, R_{9′}R_{10′}NSO₂(CH₂)ₚ or R_{9′}SO₂NR_{10′}(CH₂)ₚ, —CR_{9′}═NR_{10′}, —CR_{9′}═NOR_{10′}, —CR_{9′}═C(CN)₂, —CR_{9′}═CH(CN), R_{9′}R_{10′}N(CH₂)_{q}— and R_{9′}R_{10′}N(CH₂)_{q}O—, wherein
each R₉ and R₁₀ is independently C₁₋₄alkyl, or where appropriate R₉R₁₀ forms part of a C₃₋₆azacyloalkane or C₃₋₆(2-, 3- or 4-oxo)azacycioalkane ring
each R_{9′} and R_{10′} is independently selected from R₉ and R₁₀ and hydrogen;
each R_{9″} and R_{10′} is independently selected from R_{9′} and R_{10′} and C₁₋₄alkanoyl;
p is selected from 0, 1, 2, 3 or 4;
q is selected from 2, 3 or 4;
   wherein each R₁₄ is independently selected from hydrogen, halogen, hydroxy, cyano, amino, nitro, C₁₋₆alkyl, C₁₋₄alkoxy, haloC₁₋₄alkyl, haloC₁₋₄alkoxy, C₆₋₁₁arylC₁₋₄alkoxy, C₁₋₄alkylthio, C₃₋₆cycloalkylC₁₋₄alkyl, C₃₋₆cycloalkyl, C₃₋₆cycloalkylC₁₋₄alkoxy, C₁₋₄alkoxycarbonyl, C₁₋₄alkoxycarbonylC₁₋₄alkyl, C₁₋₄alkylsulfonyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₁₋₄alkylamido, C₁₋₄alkylsulfonamidoC₁₋₄alkyl, C₁₋₄alkylsulfinyl, C₁₋₄haloalkylsulfinyl, C₁₋₄alkylamidoC₁₋₄alkyl, C₆₋₁₁arylsulfonamido, C₄₋₉heteroarylsulfonyl, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, C₁₋₄acyl, C₆₋₁₁aryl, C₆₋₁₁arylC₁₋₄alkyl, C₁₋₄alkylaminoC₁₋₄alkyl, a group R_{9″}R_{10″}N—, R₉R₁₀NCO(CH₂)ₚ, R_{9′}R_{10′}NSO₂(CH₂)ₚ or R_{9′}SO₂NR_{10′}(CH₂)ₚ, —CR_{9′}═NR_{10′}, —CR_{9′}═NOR_{10′}, —CR_{9′}═C(CN)₂, —CR_{9′}═CH(CN), R_{9′}R_{10′}N(CH₂)_{q}— and R_{9′}R_{10′}N(CH₂)_{q}O—, wherein
each R₉ and R₁₀ is independently C₁₋₄alkyl, or where appropriate R₉R₁₀ forms part of a C₃₋₆azacyloalkane or C₃₋₆(2-, 3- or 4-oxo)azacycloalkane ring
each R_{9′} and R_{10′} is independently selected from R₉ and R₁₀ and hydrogen;
each R_{9″} and R_{10″} is independently selected from R_{9′} and R_{10′} and C₁₋₄alkanoyl;
p is selected from 0, 1, 2, 3 or 4;
q is selected from 2, 3 or 4;
   wherein each R₁₅ is independently selected from hydrogen, halogen, hydroxy, cyano, amino, nitro, C₁₋₆alkyl, C₁₋₄alkoxy, haloC₁₋₄alkyl, haloC₁₋₄alkoxy, C₆₋₁₁arylC₁₋₄alkoxy, C₁₋₄alkylthio, hydroxyC₁₋₄alkyl, C₁₋₄alkoxyC₁₋₄alkyl, C₁₋₄haloalkoxyC₁₋₄alkyl, C₃₋₆cycloalkylC₁₋₄alkyl, C₃₋₆cycloalkyl, C₃₋₆cycloalkylC₁₋₄alkoxy, C₁₋₄alkanoyl, C₁₋₄haloalkanoyl, C₁₋₄alkoxycarbonyl, C₁₋₄alkoxycarbonylC₁₋₄alkyl, C₁₋₄alkylsulfonyl, C₁₋₄haloalkylsulfonyl, C₁₋₄alkylsulfinyl, C₁₋₄haloalkylsulfinyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylamido, C₁₋₄alkylsulfonamidoC₁₋₄alkyl, C₁₋₄alkylamidoC₁₋₄alkyl, C₆₋₁₁arylsulfonamido, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, C₁₋₄acyl, C₆₋₁₁aryl, C₆₋₁₁arylC₁₋₄alkyl, C₁₋₄alkylaminoC₁₋₄alkyl, a group R_{9″}R_{10″}N—, R₉R₁₀NCO(CH₂)ₚ, R_{9′}R_{10′}NSO₂(CH₂)ₚ or R_{9′}SO₂NR_{10′}(CH₂)ₚ, —CR_{9′}═NR_{10′}, —CR_{9′}═NOR_{10′}, —CR_{9′}═C(CN)₂, —CR_{9′}═CH(CN), R_{9′}R_{10′}N(CH₂)_{q}— and R_{9′}R_{10′}N(CH₂)_{q}O—, wherein
each R₉ and R₁₀ is independently C₁₋₄alkyl, or where appropriate R₉R₁₀ forms part of a C₃₋₆azacyloalkane or C₃₋₆(2-, 3- or 4-oxo)azacycloalkane ring
each R_{9′} and R_{10′} is independently selected from R₉ and R₁₀ and hydrogen;
each R_{9″} and R_{10″} is independently selected from R_{9′} and R_{10′} and C₁₋₄alkanoyl;
p is selected from 0, 1, 2, 3 or 4;
q is selected from 2, 3 or 4;
   or Z is selected from a monocyclic or bicyclic heteroaryl group, which monocyclic heteroaryl group is or which bicyclic heteroaryl group optionally is substituted by one or more groups selected from amino, halogen, hydroxy, cyano, nitro, C₂₋₄alkyl, C₁₋₄alkoxy, haloC₁₋₄alkyl, haloC₁₋₄alkoxy, C₆₋₁₁arylC₁₋₄alkoxy, C₁₋₄alkylthio, hydroxyC₁₋₄alkyl, C₁₋₄alkoxyC₁₋₄alkyl, C₁₋₄haloalkoxyC₁₋₄alkyl, C₃₋₆cycloalkylC₁₋₄alkyl, C₃₋₆cycloalkyl, C₃₋₆cycloalkylC₁₋₄alkoxy, C₁₋₄alkanoyl, C₁₋₄haloalkanoyl, C₁₋₄alkoxycarbonyl, C₁₋₄alkoxycarbonylC₁₋₄alkyl, C₁₋₄alkylsulfonyl, C₁₋₄haloalkylsulfonyl, C₁₋₄alkylsulfinyl, C₁₋₄haloalkylsulfinyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylamido, C₁₋₄alkylsulfonamidoC₁₋₄alkyl, C₁₋₄alkylamidoC₁₋₄alkyl, C₆₋₁₁arylsulfonamido, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, C₁₋₄acyl, C₆₋₁₁aryl, C₆₋₁₁arylC₁₋₄alkyl, C₁₋₄alkylaminoC₁₋₄alkyl, a group R_{9″}R_{10″}N—, R₉R₁₀NCO(CH₂)ₚ, R_{9′}R_{10′}NSO₂(CH₂)ₚ or R_{9′}SO₂NR_{10′}(CH₂)ₚ, —CR_{9′}═NR_{10′}, —CR_{9′}═NOR_{10′}, —CR_{9′}═C(CN)₂, —CR_{9′}═CH(CN), R_{9′}R_{10′}N(CH₂)_{q}— and R_{9′}R_{10′}N(CH₂)_{q}O—, wherein
each R₉ and R₁₀ is independently C₁₋₄alkyl, or where appropriate R₉R₁₀ forms part of a C₃₋₆azacyloalkane or C₃₋₆(2-, 3- or 4-oxo)azacycloalkane ring
each R_{9′} and R_{10′} is independently selected from R₉ and R₁₀ and hydrogen;
each R_{9″} and R_{10″} is independently selected from R_{9′} and R_{10′} and C₁₋₄alkanoyl;
p is selected from 0, 1, 2, 3 or 4;
q is selected from 2, 3 or 4;

In a further aspect of the invention, there is provided a compound of formula (Ib) as hereinbefore defined or a salt or solvate thereof.

All features and preferences for formula (I) as described above apply to compounds of formula (Ib) mutatis mutandis.

In another aspect of the invention, there is provided use of a compound of formula (Ib) as hereinbefore defined or a salt or solvate thereof in the preparation of a medicament for the treatment of a disorder mediated by GlyT1.

Preferably, the disorder mediated by GlyT1 to be treated by the use or method as hereinbefore described is a psychosis, including schizophrenia, dementia and attention deficit disorders, particularly schizophrenia.

As used herein, the term “effective amount” means that amount of a drug or pharmaceutical agent that will elicit the biological or medical response of a tissue, system, animal or human that is being sought, for instance, by a researcher or clinician.

Possible formulations include those suitable for oral, sub-lingual, buccal, parenteral (for example, subcutaneous, intramuscular, or intravenous), rectal, topical and intranasal administration and in forms suitable for administration by inhalation or insufflation (either through the mouth or nose). The most suitable means of administration for a particular patient will depend on the nature and severity of the conditions being treated and on the nature of the active compound, but, where possible, oral administration is preferred.

Formulations suitable for oral administration may be provided as discrete units, such as tablets, capsules, cachets, or lozenges, each containing a predetermined amount of the active compound; as powders or granules; as solutions or suspensions in aqueous or non-aqueous liquids; or as oil-in-water or water-in-oil emulsions.

Formulations suitable for sublingual or buccal administration include lozenges comprising the active compound and, typically, a flavoured base, such as sugar and acacia or tragacanth and pastilles comprising the active compound in an inert base, such as gelatin and glycerin or sucrose and acacia.

Formulations suitable for parenteral administration typically comprise sterile aqueous solutions containing a predetermined concentration of the active compound; the solution is preferably isotonic with the blood of the intended recipient. Although such solutions are preferably administered intraveneously, they may also be administered by subcutaneous or intramuscular injection.

Formulations suitable for rectal administration are preferably provided as unit-dose suppositories comprising the active ingredient and one or more solid carriers forming the suppository base, for example, cocoa butter.

Formulations suitable for topical or intranasal application include ointments, creams, lotions, pastes, gels, sprays, aerosols and oils. Suitable carriers for such formulations include petroleum jelly, lanolin, polyethylene glycols, alcohols, and combinations thereof.

The formulations of the invention may be prepared by any suitable method, typically by uniformly and intimately admixing the active compound(s) with liquids or finely divided solid carriers, or both, in the required proportions and then, if necessary, shaping the resulting mixture into the desired shape.

For example, a tablet may be prepared by compressing an intimate mixture comprising a powder or granules of the active ingredient and one or more optional ingredients, such as a binder, lubricant, inert diluent, or surface active dispersing agent, or by moulding an intimate mixture of powdered active ingredient and inert liquid diluent.

Aqueous solutions for parenteral administration are typically prepared by dissolving the active compound in sufficient water to give the desired concentration and then rendering the resulting solution sterile and isotonic.

It will be appreciated that the precise dose administered will depend on the age and condition of the patient and the frequency and route of administration and will be at the ultimate discretion of the attendant physician. The compound may be administered in single or divided doses and may be administered one or more times, for example 1 to 4 times per day.

A proposed dose of the active ingredient for use according to the invention for oral, sub-lingual, parenteral, buccal, rectal, intranasal or topical administration to a human (of approximately 70 kg bodyweight) for the treatment of neurological and neuropsychiatric disorders mediated by a GlyT1 inhibitor, including schizophrenia, may be about 1 to about 1000 mg, preferably about 5 to about 500 mg, more preferably about 10 to about 100 mg of the active ingredient per unit dose which could be administered, for example, 1 to 4 times per day.

The invention is further illustrated by the following non-limiting examples.

Description 1.

### Methyl 2-chloro-5-(methylthio)benzoate

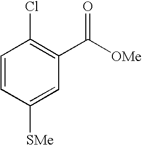

A reaction mixture of 2-chloro-5-(methylthio)benzoic acid (15.01 g, 0.07 mol) in dry methanol (180 ml) was added concentrated sulphuric acid (18 ml) at room temperature. The mixture was heated to 80° C. and left stirring at this temperature for 15 hrs. The cooled mixture was then concentrated in vacuo to give a dark brown oil. Purification by flash chromatography (SiO₂) using 5% petroleum ether (40-60° C.) in dichloromethane yielded the pure title compound as a yellow oil (9 g, 56%), d_{H} (400 MHz, CDCl₃) 7.68 (1H, d, ArH), 7.36 (1H, d, ArH), 7.29 (1H, dd, ArH), 3.94 (3H, s, OMe), 2.51 (3H, s, SMe).

Description 2.

### 2-Chloro-5-methanesulfonyl-benzoic acid methyl ester

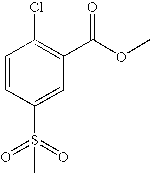

A reaction mixture of 2-chloro-5-methylsulfanyl-benzoic acid methyl ester (5.2 g, 0.02 mol) in dichloromethane (200 ml) was added portion-wise 3-chloroperoxybenzoic acid (50-55%, 24.7 g, 3 equiv.), maintaining the temperature at 25° C. by using an ice-bath. The resulting mixture was then left to stir at room temperature for 18 hrs. The insoluble white precipitate was filtered off and the cake washed with dichloromethane. The filtrate was then washed with aqueous sodium sulfite until all the oxidants have been removed. The organic layer was further washed with saturated sodium bicarbonate, dried over anhydrous magnesium sulfate, filtered and concentrated in vacuo to give the desired compound as a cream solid (4.7 g, 79%).

d_{H} (400 MHz, CDCl₃) 7.77 (1H, d, ArH), 7.46 (1H, d, ArH), 7.36 (1H, dd, ArH), 3.92 (3H, s, OMe) and 2.50 (3H, s, SMe).

Description 3.

### 2-Chloro-5-(methylsulfonyl)benzoic acid

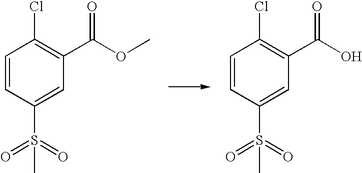

A reaction mixture of the ester (2.2 mmol), methanol (10 ml) and aqueous sodium hydroxide (10 ml, 2M) was heated to 70° C. for 18 hrs. The cooled reaction mixture was then diluted with water and ethyl acetate. The aqueous layer was acidified to pH 1 with aqueous hydrochloric acid (1 M) and extracted with ethyl acetate. The organic layer was dried over anhydrous magnesium sulfate, filtered and concentrated in vacuo to give the crude product. Purification by trituration gave the pure title compound.

Description 4.

### Methyl 5-(methylsulfonyl)-2-(1-piperidinyl)benzoate

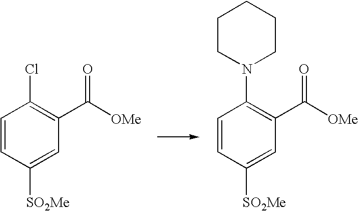

A reaction mixture of the aryl chloride (0.93 mmol, 1 equiv) and piperidine (2.32 mmol, 2.5 equiv.) in dry dimethylsulfoxide (4 ml) was heated at 80° C. for 16 hrs. The cooled mixture was then partitioned between water and dichloromethane. The aqueous layer was back extracted twice with dichloromethane. The combined organic layers were then washed with saturated brine and dried over anhydrous magnesium sulfate. Filtration followed by evaporation of the solvent in vacuo gave the crude product. Purification by flash chromatography (SiO₂), eluting with a gradient of 30-75% ethyl acetate/pentane gave the pure title compound (73%), d_{H} (400 MHz, CDCl₃) 8.20 (1H, d, ArH), 7.82 (1H, d, ArH), 7.06 (1H, dd, ArH), 3.92 (3H, s, OMe), 3.18 (4H, m, NCH₂), 3.04 (3H, s, SMe), 1.6-1.8 (6H).

Description 5.

### Methyl 5-(methylsulfonyl)-2-(4-morpholinyl)benzoate

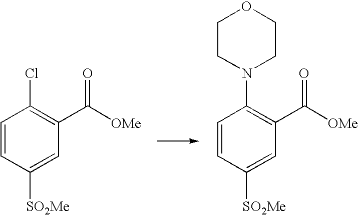

A reaction mixture of the aryl chloride (0.93 mmol, 1 equiv) and morpholine (2.32 mmol, 2.5 equiv.) in dry dimethylsulfoxide (4 ml) was heated at 80° C. for 16 hrs. The cooled mixture was then partitioned between water and dichloromethane. The aqueous layer was back extracted twice with dichloromethane. The combined organic layers were then washed with saturated brine and dried over anhydrous magnesium sulfate. Filtration followed by evaporation of the solvent in vacuo gave the crude product. Purification by flash chromatography (SiO₂), eluting with a gradient of 30-75% ethyl acetate/pentane gave the pure title compound as a white solid (75%), LC/MS (ES+) Found 300 (M+1).

Description 6.

### 5-Methanesulfonyl-2-piperidin-1-yl-benzoic acid

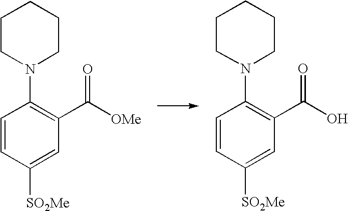

Hydrolysis of the ester by the procedure described in Description 3 gave the title compound as an off-white solid, (72%), d_{H} (400 MHz, CDCl₃) 8.87 (1H, d, ArH), 8.17 (1H, dd, ArH), 7.61 (1H, d, ArH), 3.11 (3H, s, SMe), 3.05 (4H, broad t, NCH₂), 1.91 (4H, m), 1.72 (2H, broad res.).

Description 7.

### 5-(Methylsulfonyl)-2-(4-morpholinyl)benzoic acid

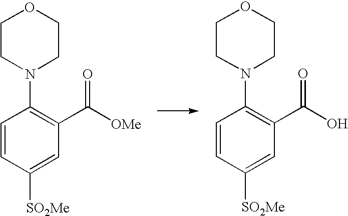

Hydrolysis of the ester by the procedure described in Description 3 gave the title compound as an off-white solid (75%), d_{H} (400 MHz, CDCl₃) 15.80 (1H, broad res.), 8.88 (1H, d, ArH), 8.21 (1H, dd, ArH), 7.63 (1H, d, ArH), 4.00 (4H, t), 3.11 (7H, s+m)

Description 8.

### 5-Methanesulfonyl-2-propylamino-benzoic acid methyl ester

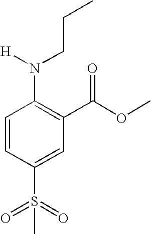

Reaction of 2-Chloro-5-methanesulfonyl-benzoic acid methyl ester and n-propylamine, following the procedure of Description 5 provided the title compound,

d_{H} (400 MHz, CDCl₃) 8.50 (1H, d, ArH), 8.40 (1H, br s, NH), 7.83 (1H, dd, ArH), 6.75 (1H, d, ArH), 3.90 (3H, OMe), 3.23 (2H, t), 3.05 (3H, s, SQ₂Me), 1.78 (2H, septet) and 1.05 (3H, t).

LC/MS (ammonium bicarbonate ESI) Found 270 (M−1, T_{R} 1.30 min)

Description 9.

### 2-(4,4-Dimethyl-piperidin-1-yl)-5-methanesulfonyl-benzoic acid methyl ester

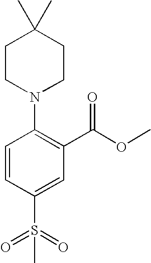

Reaction of 2-Chloro-5-methanesulfonyl-benzoic acid methyl ester and 4,4-dimethylpiperidine, following the procedure of Description 5 provided the title compound,

d_{H} (400 MHz, CDCl₃) 8.25 (1H, d, ArH), 7.85 (1H, dd, ArH), 7.02 (1H, d, ArH), 3.90 (3H, OMe), 3.17 (4H, m), 3.05 (3H, s, SO₂Me), 1.50 (4H, m) and 1.00 (6H, s, 2×Me).

LC/MS (Formic APCI) Found 326 (M+1, T_{R} 1.96 min)

Description 10.

### 5-Methanesulfonyl-2-(1,3,3-trimethyl-6-aza-bicyclo[3.2.1]oct-6-yl)-benzoic acid methyl ester

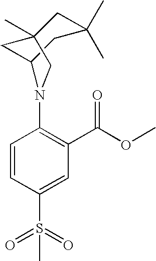

The title compound was prepared from 2-chloro-5-methanesulfonyl-benzoic acid methyl ester and 1,3,3-trimethyl-6-azabicyclo[3.2.1]octane by the procedure described in Description 5.

Description 11.

### 5-Methanesulfonyl-2-(methyl-propyl-amino)-benzoic acid methyl ester

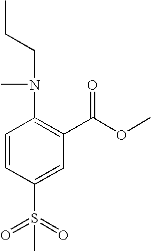

The title compound was prepared from 2-chloro-5-methanesulfonyl-benzoic acid methyl ester and methyl(propyl)amine by the procedure described in Description 5.

d_{H} (400 MHz, CDCl₃) 8.15 (1H, d, ArH), 7.78 (1H, dd, ArH), 6.96 (1H, d, ArH), 3.90 (3H, OMe), 3.23 (2H, t), 3.05 (3H, s, SQ₂Me), 2.92 (3H, s, NMe), 1.68 (2H, septet) and 0.95 (3H, t).

LC/MS (ammonium bicarbonate ESI) Found 286 (M+1, T_{R} 1.88 min)

Description 12.

### 5-Methanesulfonyl-2-(2-methyl-piperidin-1-yl)-benzoic acid methyl ester

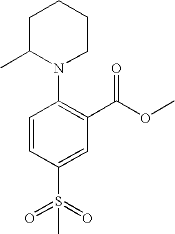

The title compound was prepared from 2-chloro-5-methanesulfonyl-benzoic acid methyl ester and 2-methylpiperidine by the procedure described in Description 5.

d_{H} (400 MHz, CDCl₃) 8.18 (1H, br s, ArH), 7.85 (1H, d, ArH), 7.07 (1H, d, ArH), 3.90 (3H, OMe), 3.76 (1H, m), 3.45 (3H, m), 3.25 (1H, m) 3.05 (3H, s, SO₂Me), 1.87 (1H, m), 1.65 (3H, m) and 1.10 (3H, d).

LC/MS (Formic APCI) Found 312 (M+1, T_{R} 1.52 min)

Description 13.

### 5-Methanesulfonyl-2-propylamino-benzoic acid

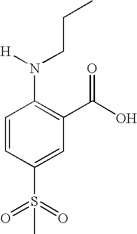

Hydrolysis of the methyl ester by the procedure of Description 3 provided the title compound.

d_{H} (400 MHz, CDCl₃) 8.55 (1H, d, ArH), 8.29 (1H, br s, NH), 7.83 (1H, dd, ArH), 6.75 (1H, d, ArH), 3.23 (2H, t), 3.05 (3H, s, SO₂Me), 1.73 (2H, septet) and 1.00 (3H, t).

LC/MS (Formic APCI) Found 258 (M+1, T_{R} 1.39 min)

Description 14.

### 2-(2,6-Dimethyl-morpholin-4-yl)-5-methanesulfonyl-benzoic acid

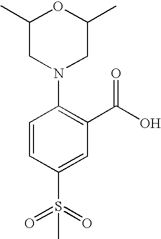

Hydrolysis of the methyl ester by the procedure of Description 3 provided the title compound, isolated as a pale pink solid.

d_{H} (400 MHz, CDCl₃) 8.83 (1H, d, ArH), 8.20 (1H, dd, ArH), 7.60 (1H, d, ArH), 3.90 (2H, m), 3.12 (3H, s, SO₂Me), 2.97 (2H, d), 2.78 (2H, m) and 1.30 (6H, d, 2×Me).

LC/MS (Formic APCI) Found 314 (M+1, T_{R} 0.92 min).

Description 15.

### 2-(3,5-Dimethyl-piperidin-1-yl)-5-methanesulfonyl-benzoic acid

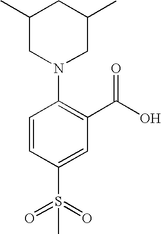

Hydrolysis of the methyl ester by the procedure of Description 3 provided the title compound, isolated as an off-white solid.

d_{H} (400 MHz, CDCl₃) 8.85 (1H, d, ArH), 8.18 (1H, dd, ArH), 7.60 (1H, d, ArH), 3.12 (3H, s, SO₂Me), 3.05 (2H, d), 2.45 (2H, t), 3.00 (3H, m), 1.12 (1H, m), 1.00 (6H, d, 2×Me) and 0.85 (1H, m).

LC/MS (Formic APCI) Found 312(M+1, T_{R} 1.04 min).

Description 16.

### 2-(4,4-Dimethyl-piperidin-1-yl)-5-methanesulfonyl-benzoic acid

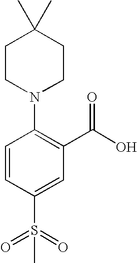

Hydrolysis of the methyl ester by the procedure of Description 3 provided the title compound.

d_{H} (400 MHz, CDCl₃) 8.87 (1H, d, ArH), 8.18 (1H, dd, ArH), 7.65 (1H, d, ArH), 3.10 (3H, s, SO₂Me), 3.10-2.95 (4H, m), 1.80-1.75 (4H, m), 1.12 (6H, s, 2×Me).

LC/MS (Formic APCI) Found 312 (M+1, T_{R} 0.89 min).

Description 17.

### 5-Methanesulfonyl-2-(1,3,3-trimethyl-6-aza-bicyclo[3.2.1]oct-6-yl)-benzoic acid

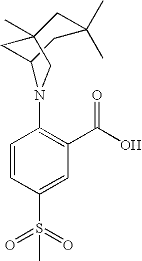

Hydrolysis of the methyl ester by the procedure of Description 3 provided the title compound.

d_{H} (400 MHz, CDCl₃) 8.20 (1H, s, ArH), 7.76 (1H, d, ArH), 6.85 (1H, d, ArH), 4.27 (1H, m), 3.36 (1H, d), 3.05 (3H, s, SO₂Me), 2.95 (1H, d), 2.15 (1H, d), 1.92 (1H, m), 1.50 (5H, m), 1.15 (3H, s), 0.91 (3H, s) and 0.75 (3H, s).

LC/MS (Formic APCI) Found 352 (M+1, T_{R} 1.78 min)

Description 18.

### 5-Methanesulfonyl-2-(methyl-propyl-amino)-benzoic acid

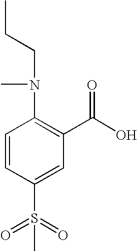

Hydrolysis of the methyl ester by the procedure of Description 3 provided the title compound.

d_{H} (400 MHz, CDCl₃) 8.88 (1H, d, ArH), 8.20 (1H, dd, ArH), 7.63 (1H, d, ArH), 3.10 (3H, s, SO₂Me), 3.00 (2H, t), 2.82 (3H, s, NMe), 1.55 (2H, septet) and 0.95 (3H, t).

LC/MS (Formic APCI) Found 272 (M+1, T_{R} 0.56 min)

Description 19.

### 1-[3-Fluoro-4-(1-piperazinyl)phenyl]ethanone

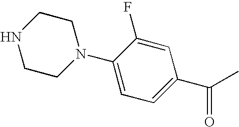

A solution of 3′,4′-difluoroacetophenone (14.3 g, 0.092 mol) in acetonitrile (150 ml) was treated with piperazine (27.6 g, 0.32 mol) and heated at reflux for 18 h. The mixture was allowed to cool and partitioned between EtOAc and water. The organics were further washed with water (×2), dried (Na₂SO₄) and evaporated in vacuo to the title compound (17.6 g, 86%).

LC/MS Found 223 (ESI) (M+1)

Description 20.

### Methyl 2-(4,4-difluoro-1-piperidinyl)-5-(methylsulfonyl)benzoate

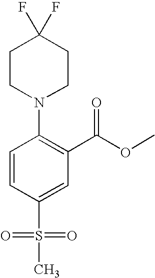

A mixture of methyl 2-chloro-5-(methylsulfonyl)benzoate (1.57 g), DIPEA (1 ml), potassium carbonate (200 mg) and 4,4-difluoropiperidine hydrochloride (1.0 g) in DMSO (5 ml) was heated at 80° C. for 2 days. After cooling, the reaction mixture was partitioned between ethyl acetate and diluted hydrochloride solution. The organic solution was washed with water, dried (Na₂SO₄) and evaporated, the residue was chromatographed over silica gel. Elution with a gradient of 10 to 100% ethyl acetate in N-pentane gave the title compound as a white solid (22% yield).

LC/MS (ESI) Found 334 (M+1).

Description 21.

### 2-(4,4-Difluoro-1-piperidinyl)-5-(methylsulfonyl)benzoic acid

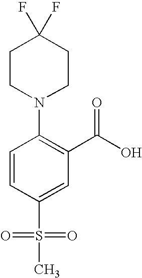

The title compound was prepared from methyl 2-(4,4-difluoro-1-piperidinyl)-5-(methylsulfonyl)benzoate according to the procedure described in Description 3.

Description 23.

### 2-Chloro-5-(chlorosulfonyl)benzoic acid

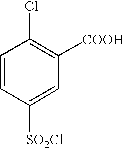

Chlorosulphonic acid (10 ml) was stirred at 0° during the addition of o-chlorobenzoic acid (4.69 g) added portionwise over 5 mins. Stirred at ambient temperature overnight. The reaction mixture was added dropwise to stirred ice and the resulting white solid collected by filtration. Dried to give the desired compound (6.1 g, 79%).

Description 24.

### 2-Chloro-5-(dioxidosulfanyl)benzoic acid

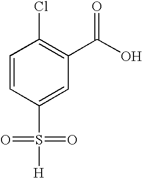

2-Chloro-5-(chlorosulfonyl)benzoic acid (3.0 g) was added in portions to a stirred suspension of sodium sulphite (3.24 g) in water (6 ml). The pH was then adjusted to 9 by addition of 50% sodium hydroxide solution and stirred for 18 hours at room temperature. The solution was acidified to pH 1 by addition of 5M HCl and after 10 minutes the suspension was cooled in ice and the solid collected by filtration. Obtained desired product as a white solid (2.43 g, 94%).

Description 25.

### Ethyl 2-chloro-5-(ethylsulfonyl)benzoate

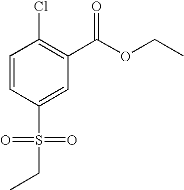

2-Chloro-5-(dioxidosulfanyl)benzoic acid (1.0 g) in DMF (3 ml) was treated with potassium carbonate (1.86 g) and iodoethane (1.1 ml) and stirred for 18 hours. The reaction mixture was then diluted with ethyl acetate and washed 6 times with water, dried and evaporated to afford crude product. Chromatography on silica gel eluting with 5-50% ethyl acetate in pentane afforded the diethyl derivative as a colourless oil (1.0 g, 80%). LC/MS Found 249 (ESI) (MH-28).

Description 26.

### Ethyl 5-(ethylsulfonyl)-2-(1-piperidinyl)benzoate

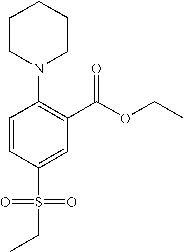

A mixture of ethyl 2-chloro-5-(ethylsulfonyl)benzoate (1.0 g) and piperidine (705 ul) in DMSO (3 ml) was heated to 80° for 18 hours. The cooled solution was diluted with ethyl acetate, washed with sat. NaHCO3 solution and water (5 times), dried and evaporated to yield the desired product as a yellow oil (1.18 g, 100%). LC/MS Found 326 (ESI) (M+1).

Description 27.

### 5-(Ethylsulfonyl)-2-(1-piperidinyl)benzoic acid

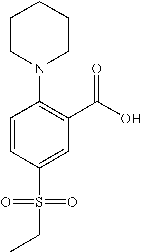

A solution of the ethyl ester (1.18 g) in ethanol (10 ml) containing 2M sodium hydroxide solution (5.4 ml) was heated to 70° for 18 hours. The volatile components were removed under reduced pressure and the residue washed with ethyl acetate. The aqueous layer was acidified by addition of 2M HCl and extracted with ethyl acetate (3×), dried and evaporated to afford the desired product as a white solid (806 mg, 75%).

Description 28.

### 5-[(1-Methylethyl)sulfonyl]-2-(1-piperidinyl)benzoic acid

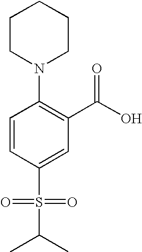

The title compound was prepared by an analogous method to that given for 5-(ethylsulfonyl)-2-(1-piperidinyl)benzoic acid (Description 27). Desired compound obtained as a white solid (0.28 g, 70%).

Description 29.

### Methyl 2-chloro-5-(methylsulfinyl)benzoate

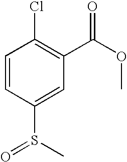

A solution of methyl 2-chloro-5-(methylthio)benzoate (0.40 g) in DCM (10 ml) at 0° was treated with 75% m-chloroperbenzoic acid (420 mg). Stirred at ambient temperature for 4 hours. The reaction mixture was washed with 10% sodium carbonate solution, dried and evaporated to afford crude sulphoxide as a yellow solid (406 mg, 96%). LC/MS Found 233 (ESI) (M+1).

Description 30.

### Methyl 5-(methylsulfinyl)-2-(1-piperidinyl)benzoate

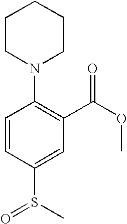

A solution of methyl 2-chloro-5-(methylsulfinyl)benzoate (200 mg) in DMSO (1 ml) containing piperidine (0.5 ml) was heated at 800 for 48 hours. The solvent was then removed under reduced pressure and the residue dissolved in ethyl acetate. Washed with saturated sodium bicarbonate and evaporated to afford crude product which was chromatographed on silica gel. Elution with 50-90% ethyl acetate in pentane gave the desired product as a gum (27 mg, 11%). LC/MS Found 282 (ESI) (M+1).

Description 31.

### 5-(Methylsulfinyl)-2-(1-piperidinyl)benzoic acid

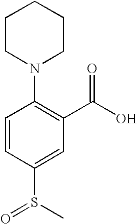

A solution of methyl 5-(methylsulfinyl)-2-(1-piperidinyl)benzoate (27 mg) in methanol (200 ul) was treated with 1 equivalent of 2M sodium hydroxide solution and heated at 700 for 18 hours. On cooling, an excess of 1M HCl in ether was added and the mixture evaporated to afford the crude product as a solid. This was used without further purification. LC/MS Found 290 (ESI) (M+Na).

Description 32.

### 1,1-Dimethylethyl 4-(4-acetyl-3,5-dichlorophenyl)-1-piperazinecarboxylate

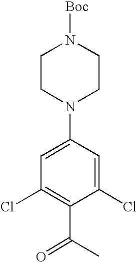

A solution of 1.4M methylmagnesium bromide in THF (29 ml) was stirred under argon at 500 and a solution of 2,4,6-trichlorobenzoyl chloride (2.00 g) in dry toluene (2 ml) was added over 15 minutes. Stirring was continued for a further 30 minutes and the cooled (0°) reaction mixture was then treated with ice and diluted with ether and 2M hydrochloric acid. Organic layer dried and evaporated to afford 2,4,6-trichloroacetophenone as an oil (1.78 g). This material (1.0 g), N-Boc piperazine (0.83 g), caesium carbonate (2.18 g), palladium acetate (101 mg) and BINAP (418 mg) were heated in 1,4-dioxane (30 ml) at 100° under argon for 24 hours. After cooling, the precipitate was removed by filtration and the filtrate evaporated at reduced pressure. The residue was partitioned between ethyl acetate and water and the aqueous layer extracted with further ethyl acetate. The combined organic layers were dried and evaporated. Chromatography on silica gel eluting with 0-50% ethyl acetate/pentane gave desired product as a yellow gum (81 mg).

LC/MS (ESI) found 273 (MH-100).

Description 33.

### 1-[2,6-Dichloro-4-(1-piperazinyl)phenyl]ethanone

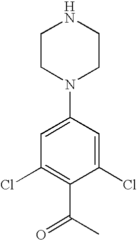

1,1-dimethylethyl 4-(4-acetyl-3,5-dichlorophenyl)-1-piperazinecarboxylate (81 mg) was treated with trifluoroacetic acid (0.9 ml) and water (0.1 ml) and stirred for 1 hour. The volatile components were removed under reduced pressure to afford the desired product as a brown gum.

Description 34.

### 1-[4-(1-piperazinyl)-2-(trifluoromethyl)phenyl]ethanone

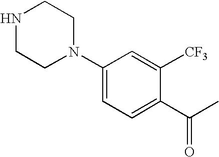

A solution of 1-[4-fluoro-2-(trifluoromethyl)phenyl]ethanone (4.83 g, 0.023 mol) in acetonitrile (150 ml) was treated with piperazine (7.1 g, 0.082 mol) and the mixture heated at reflux under argon for 20 h. On cooling the mixture was partitioned between water and EtOAc. The organics were washed further with water (×2), dried (Na₂SO₄) and evaporated in vacuo to a yellow solid 5.8 g, 93%.

δ_{H} (400 MHz, CDCl₃) 7.55 (1H, d), 7.18 (1H, d), 6.96 (1H, dd), 3.30 (4H, m), 3.03 (4H, m), 2.54 (3H, s).

LC/MS (ESI) Found 273 (M+1)

Description 35.

### 1-[2-Chloro-4-(1-piperazinyl)phenyl]ethanone

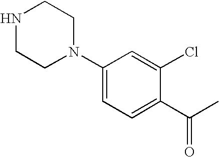

The compound was prepared from 1-(2-chloro-4-fluorophenyl)ethanone (4.8 g, 0.028 mol) by the procedure described in Description 34 to afford the title compound 6.4 g, 96%.

LC/MS (ESI) Found 239/241 (M+1)

Description 37.

### 1-[6-(1-piperazinyl)-3-pyridinyl]ethanone

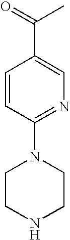

The title compound was prepared from 1-(6-chloro-3-pyridinyl)ethanone and piperazine according to the procedure described in J. Med. Chem. 1999, Vol 42, No:14 p. 2577. (90% yield) LC/MS (ESI) Found 206.2 (M+1)

Description 38.

### 1-{[2-Chloro-5-(methylsulfonyl)phenyl]carbonyl}-4-[3,5-dichloro-4-(methyloxy)phenyl]piperazine

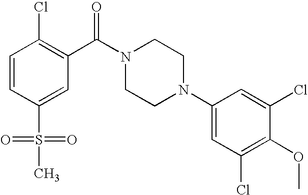

The title compound was prepared from 2-chloro-5-(methylsulfonyl)benzoic acid and 1-[3,5-dichloro-4-(methyloxy)phenyl]piperazine hydrochloride according to the procedure described in Example 92. (87% yield). LC/MS (ESI) Found 478.9 (M+1).

Description 39.

### 4-[4-(Methylthio)phenyl]piperazine-1-carboxylic acid-tert-butylester

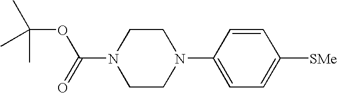

Under an inert atmosphere of argon, sodium tert-butoxide (200 mg; 2.08 mmol) was added in one portion to a room temperature stirring solution of N-Boc-piperazine (199 mg; 1.07 mmol), 1-bromo-4-(methylthio)benzene (217 mg; 1.07 mmol), 2-dicyclohexylphosphino-2′-(N,N-dimethylamino)biphenyl (113 mg; 0.29 mmol), and Pd₂(dba)₂ (57 mg; 62 μmol) in degassed 1,4-dioxan (5 mL). The mixture was sonicated for 1 minute, and stirred under microwave conditions in a sealed tube at 100° C. for 5 minutes. The crude mixture was partitioned between ethyl acetate (40 mL) and water (40 mL), and the separated aqueous extracted with ethyl acetate (40 mL). The combined organic phase was dried (MgSO₄), and concentrated in vacuo. The resulting brown oil was purified by column chromatography, giving the title compound 4-[4-(methylthio)phenyl]piperazine-1-carboxylic acid-tert-butylester as a yellow solid (259 mg; 79%). LC/MS (Ammonium bicarbonate ES+) Found 209 (M-Boc+H).

Description 40.

### 1-[4-(Methylthio)phenyl]piperazine

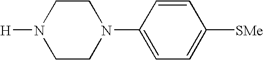

Trifluoroacetic acid (200 μL; 2.69 mmol) was added in one portion to a room temperature stirring solution of carbamate 4-[4-(methylthio)phenyl]piperazine-1-carboxylic acid-tert-butylester (79 mg; 0.26 mmol) in dichloromethane (1 mL). The reaction was stirred for 15 hours and purified using an SCX ion exchange column, giving 1-[4-(methylthio)phenyl]piperazine as a pale yellow solid (42.9 mg; 79%).

Description 41.

### 4-[4-(Methylsulfinyl)phenyl]piperazine-1-carboxylic acid-tert-butylester

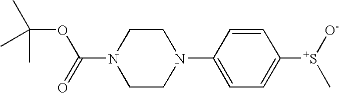

meta-Chloroperoxybenzoic acid (approx. 77%; 191 mg; 0.85 mmol) was added in one portion to a cool (0° C.) stirring solution of 4-[4-(methylthio)phenyl]piperazine-1-carboxylic acid-tert-butylester (179 mg; 0.58 mmol) in dichloromethane (3 mL). The mixture was stirred at 0° C. for 1% hours, quenched with saturated aqueous sodium hydrogen carbonate (5 mL) and diluted with water (1 mL) and dichloromethane (2 mL). The separated aqueous phase was extracted with dichloromethane (10 mL), and the combined organic phase dried (MgSO₄). Concentration in vacuo gave a yellow solid (189 mg) which was purified by column chromatography giving 4-[4-(methylsulfinyl)phenyl]piperazine-1-carboxylic acid-tert-butylester (141 mg; 75%) as a pale yellow solid. LC/MS (Ammonium bicarbonate ES+) Found 347 (M+Na).

Description 42.

### 4-[4-(methylsulfinyl)phenyl]piperazine-1-carboxylic acid-tert-butylester

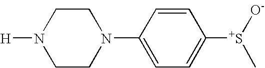

Trifluoroacetic acid (250 μL; 3.37 mmol) was added in one portion to a room temperature stirring solution of 4-[4-(methylsulfinyl)phenyl]piperazine-1-carboxylic acid-tert-butylester (141 mg; 0.436 mmol) in dichloromethane (1.7 mL). The reaction was stirred for 18 hours and purified using an SCX ion exchange column, giving 4-[4-(methylsulfinyl)phenyl]piperazine-1-carboxylic acid-tert-butylester as a white solid (92.9 mg; 93%). LC/MS (Ammonium bicarbonate ES+) Found 225 (M+H).

Description 43.

### 1,1-Dimethylethyl 4-(5-nitro-2-pyridinyl)-1-piperazinecarboxylate

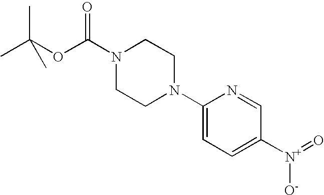

N—BOC piperazine (0.5 g) and 2-chloro-5-nitropyridine (0.424 g) in DMF (16 ml) were treated with potassium carbonate (0.744 g) and DIPEA (1.41 ml). The resulting mixture was heated at 120° C. for 4 hours. After cooling to room temperature, the solvent was removed in vacuo and the residue partitioned between ethyl acetate and water. Organic layer washed again with water then dried over anhydrous magnesium sulphate. After filtration, the solvent evaporated to dryness in vacuo to afford the title compound as a pale brown solid in 95%.

LCMS (ES+) 209.05 (MH+—BOC)

Description 44.

### 1-(5-Nitro-2-pyridinyl)piperazine

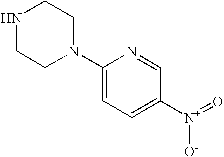

Compound from Description 43 (0.78 g) was dissolved in dry DCM (18 ml), cooled in an ice bath to ˜0° C. before adding TFA (2 ml) slowly over 5 minutes. The resultant solution was then allowed to warm up to R.T. and was stirred for 4 hours, under argon. The reaction solution was poured slowly onto ice saturated potassium carbonate before extracting with DCM (3×50 ml). The combined extracts were dried over anhydrous magnesium sulphate, filtered and solvent removed in vacuo to afford the title compound as a yellow solid in 71%.

1H NMR (400 MHz, CDCl3) δ 9.03 (d, 1H), 8.20 (dd, 1H), 6.55 (d, 1H), 3.75 (m, 4H), 2.98 (m, 4H)

Description 45.

### 1,1-Dimethylethyl 4-(6-chloro-4-pyrimidinyl)-1-piperazinecarboxylate

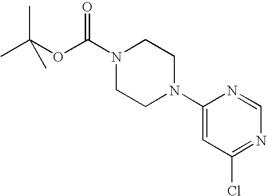

The title compound was prepared by the method as Description 43, heating for 16 hours to afford the title compound as a pale orange solid in 94% yield.

LCMS (ES+) 299 (M+H)

Description 46.

### 4-Chloro-6-(1-piperazinyl)pyrimidine

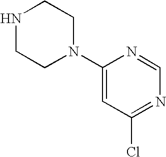

The title compound, obtained as an orange oil, was prepared in a similar manner to Description 44 (95% yield).

LCMS (ES+) 199 (M+H)

Description 47.

### 1,1-Dimethylethyl 4-(3-acetylphenyl)-1-piperazinecarboxylate

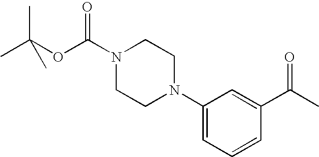

N—BOC piperazine (372 mg), 3-bromoacetophenone (398 mg), palladium acetate (45 mg), rac-BINAP (79 mg), and sodium t-butoxide (288 mg) were heated in THF (7 ml) at 85° C. for 28 hours. After cooling to rt, the reaction mixture was filtered through Kieselguhr, washing well with water and ethyl acetate. The layers were separated. The organic layer was dried over anhydrous magnesium sulphate. The solution was filtered and the solvent was removed in vacuo. The residue purified by silica gel chromatography, eluting with ethyl acetate/pentane mixtures. The desired fractions were combined and solvent removed in vacuo to afford the title compound as a yellow gum in 7% yield.

LCMS (ES+) 205 (M+H—BOC)

Description 48.

### 1-[3-(1-piperazinyl)phenyl]ethanone

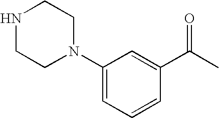

The BOC piperazine derivative from Description 47 (100 mg), was dissolved in methanol (3 ml) and treated with 4M HCl in dioxane (1 ml). the resulting solution was stirred for 18 hours at rt under an atmosphere of argon. The solvent removed in vacuo to afford the title compound as an orange gummy solid in quantitative yield.

LCMS (ES+) 205 (M+H)

Description 49.

### 1,1-Dimethylethyl 4-(1H-pyrrolo[2,3-b]pyridin-4-yl)-1-piperazinecarboxylate

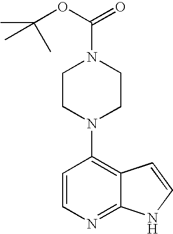

A solution of 4-bromo-1H-pyrrolo[2,3-b]pyridine (6.1 g) and N-butyloxycarbonylpiperazine (29.8 g) in N-methylpyrrolidine (20 ml) was heated on an oil bath at 135° under argon for 3 days. The mixture was dissolved in DCM (500 ml) and washed with water (6×250 ml), dried and concentrated under reduced pressure. Treatment with ether (200 ml) afforded a cream solid (7.1 g).

Description 50.

### 1-(Phenylsulfonyl)-4-(1-piperazinyl)-1H-pyrrolo[2,3-b]pyridine

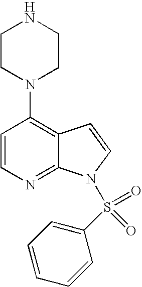

A solution of 1,1-dimethylethyl 4-(1H-pyrrolo[2,3-b]pyridin-4-yl)-1-piperazinecarboxylate (100 mg) in THF (1 ml) and DMF (1 ml) was treated with BEMP (0.19 ml). A solution of phenylsulphonyl chloride (76 mg) in THF (1 ml) was then added and the solution stirred under argon for 2.5 hours. After dilution with ethyl acetate, the reaction mixture was washed with 2M HCl and brine, dried and concentrated to a yellow oil. This was chromatographed on silica gel eluting with 10-50% ethyl acetate in pentane to give an oil (34 mg). This was treated with trifluoroacetic acid for 1 hour and the volatile components removed under reduced pressure to give the desired product as an oil (30 mg).

Description 51.

### 1-(2-Furanylsulfonyl)-4-(1-piperazinyl)-1H-pyrrolo[2,3-b]pyridine

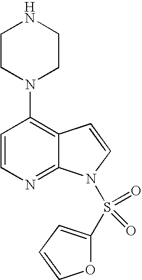

Prepared as described for 1-(phenylsulfonyl)-4-(1-piperazinyl)-1H-pyrrolo[2,3-b]pyridine (Description 50).

Description 52.

### (2-Chloro-5-methanesulfonyl-phenyl)-[4-(3,4-dichloro-phenyl)-piperazin-1-yl]-methanone

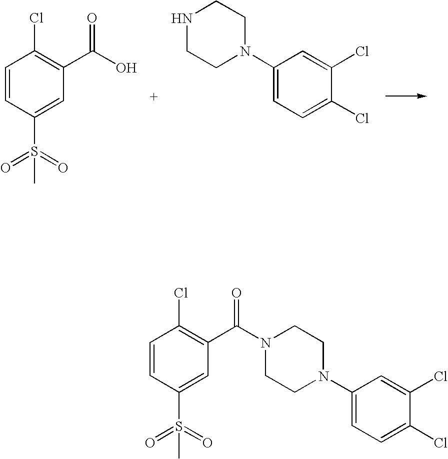

Reaction of 2-chloro-5-methanesulfonylbenzoic acid and 1-(3,4-dichlorophenyl)piperazine by the procedure described in Example 5 provided the title compound, isolated as a beige solid.

### EXAMPLE 1

### [[4-(3,4-Dichloro-phenyl)-piperazin-1-yl]-(5-methanesulfonyl-2-morpholin-4-yl-phenyl)-methanone

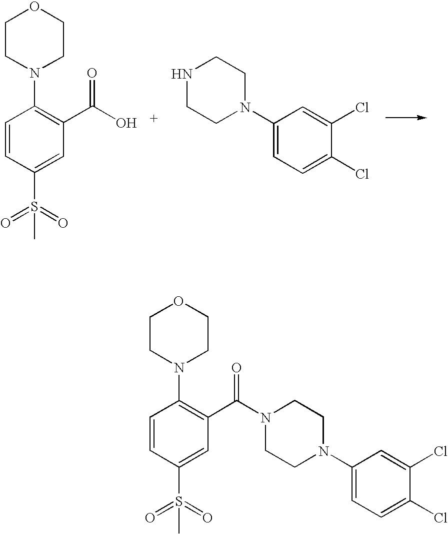

A reaction mixture of acid (1 equiv.), HBTU (1 equiv.) and diisopropylamine (2 equiv.) in 5:2 tetrahydrofuran/N,N-dimethylformamide (0.1M) was stirred for 30 minutes at room temperature under nitrogen atmosphere. The piperazine (1.1 equiv.) was then added and the resulting mixture was heated at 80° C. for 16 hrs. The cooled reaction mixture was concentrated in vacuo to give a residue, which was then re-dissolved in ethyl acetate. The organic solution was washed with water, followed by saturated brine. The organic layer was then dried over anhydrous magnesium sulfate, filtered and concentrated in vacuo to give the crude product. Purification by flash chromatography (SiO₂) afforded the desired product as a white solid, d_{H} (400 MHz, CDCl₃) 7.92 (1H, dd, ArH), 7.85 (1H, d, ArH), 7.31 (1H, d, ArH), 7.10 (1H, d, ArH), 6.95 (1H, d, ArH), 6.74 (1H, dd, ArH), 4.15 (1H, br m), 3.80 (5H, m), 3.50-3.15 (7H, m), 3.04 (3H, S, SO₂Me) and 3.03-2.95 (3H, m).

LC/MS (Ammonium bicarbonate APCI) Found 498/500 (M+1, T_{R} 2.11 min).

### EXAMPLE 2

### [4-(4-Chloro-3-nitro-phenyl)-piperazin-1-yl]-(5-methanesulfonyl-2-morpholin-4-yl-phenyl)-methanone

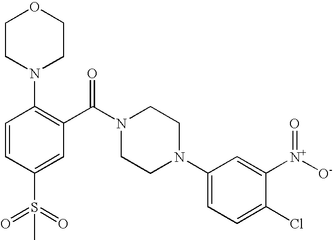

The title compound was prepared from 5-(methylsulfonyl)-2-(4-morpholinyl)benzoic acid and the piperazine using the procedure of Example 1,

d_{H} (400 MHz, CDCl₃) 7.92 (1H, dd, ArH), 7.85 (1H, d, ArH), 7.40 (1H, d, ArH), 7.34 (1H, d, ArH), 7.10 (1H, d, ArH), 7.00 (1H, dd, ArH), 4.20 (1H, br m), 3.75 (5H, m), 3.50-3.20 (7H, m), 3.05 (3H, s, SO₂Me) and 3.10-2.90 (3H, m (excl. Me)).

LC/MS (Ammonium bicarbonate ESI) Found 509 (M+1, T_{R} 1.97 min)

### EXAMPLE 3

### [4-(3,5-Dichloro-4-methoxy-phenyl)-piperazin-1-yl]-(5-methanesulfonyl-2-morpholin-4-yl-phenyl)-methanone

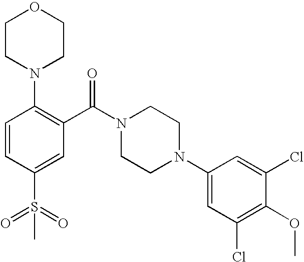

A reaction mixture of 5-methanesulfonyl-2-morpholin-4-yl-benzoic acid (110 mg, 0.38 mmol), HBTU (150 mg, 0.40 mmol) and diisopropylamine (0.26 ml, 1.5 mmol) in dry tetrahydrofuran (10 ml) and dry N,N-dimethylformamide (1.2 ml) was stirred for 15 minutes at room temperature under nitrogen atmosphere. 1-(3,5-Dichloro-4-methoxy-phenyl)-piperazine hydrochloride (125 mg, 0.42 mmol) was then added and the resulting mixture was stirred at room temperature for 16 hrs. The reaction mixture was concentrated in vacuo and water (100 ml) was added to the residue obtained. The mixture was left stirring for 30 minutes and the resulting white precipitate was then extracted with ethyl acetate. The organic layer was washed with saturated brine, dried over anhydrous magnesium sulfate, filtered and concentrated in vacuo to give the crude product. Purification by flash chromatography (SiO₂) afforded the desired product as a white solid.

d_{H} (400 MHz, CDCl₃) 7.93 (1H, dd, ArH), 7.83 (1H, d, ArH), 7.09 (1H, d, ArH), 6.82 (2H, s, 2×ArH), 4.14 (1H, ddd), 3.84 (3H, s, OMe), 3.80-3.70 (5H, br m), 3.40 (3H, br m), 3.27 (2H, m), 3.15 (2H, m), 3.05 (3H, s, SO₂Me) and 3.04-2.90 (3H, m).

LC/MS (Ammonium bicarbonate APCI) Found 528/530 (M+1, T_{R} 2.08 min)

### EXAMPLE 4

### 2,2,2-Trifluoro-1-{3-fluoro-4-[4-(5-methanesulfonyl-2-morpholin-4-yl-benzoyl)-piperazin-1-yl]-phenyl}-ethanone and {4-[2-Fluoro-4-(2,2,2-trifluoro-1,1-dihydroxy-ethyl)-phenyl]-piperazin-1-yl}-(5-methanesulfonyl-2-morpholin-4-yl-phenyl)-methanone

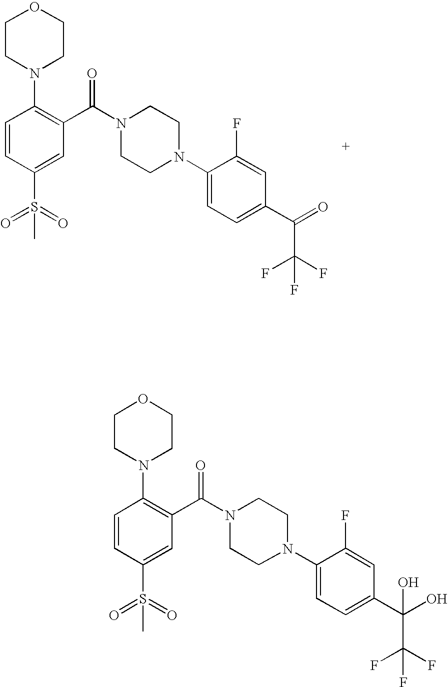

A reaction mixture of 5-methanesulfonyl-2-morpholin-4-yl-benzoic acid (70 mg, 0.25 mmol), HBTU (95 mg, 0.25 mmol) and diisopropylamine (0.1 ml, 0.5 mmol) in dry tetrahydrofuran (5 ml) and dry N,N-dimethylformamide (4 drops) was stirred for 10 minutes at room temperature under nitrogen atmosphere. 2,2,2-Trifluoro-1-(3-fluoro-4-piperazin-1-yl-phenyl)-ethanone (75 mg, 0.27 mmol) was then added to the mixture and after 2 days of stirring, the reaction mixture was concentrated in vacuo to give a yellow residue. The residue was then dissolved in ethyl acetate and washed with water, followed by saturated brine. The organic layer was then dried over anhydrous magnesium sulfate, filtered and concentrated in vacuo to give the crude product. Purification by flash chromatography (SiO₂) afforded the desired product as a yellow solid.

d_{H} (400 MHz, CDCl₃) 7.93 (1H, dd, ArH), 7.87 (1H, d, ArH), 7.82 (1H, d, ArH), 7.75 (1H, d, ArH), 7.12 (1H, br d, ArH), 6.93 (1H, t, ArH), 4.29 (1H, br m), 3.90-2.90 (15H, m) and 3.06 (3H, s);

LC/MS (Formic APCI) Found 544 (M+1, T_{R} 1.90 min) and 562 (M+H₂O+1, T_{R} 1.37 min)

### EXAMPLE 5

### (5-Methanesulfonyl-2-morpholin-4-yl-phenyl)-[4-(5-trifluoromethyl-pyridin-2-yl)-piperazin-1-yl]-methanone

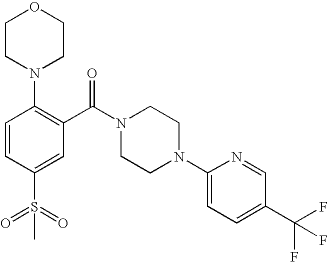

A reaction mixture of 1-(5-trifluoromethyl-pyridin-2-yl)-piperazine (32 mg, 0.14 mmol), 5-methanesulfonyl-2-morpholin-4-yl-benzoic acid (35.5 mg, 0.12 mmol) and EDCI (52 mg, 0.27 mmol) in dry dichloromethane (6 ml) was stirred at room temperature for 2 hrs. TLC indicated no reaction. The reaction mixture was therefore heated to 35° C. for 4 hrs. TLC indicated consumption of starting materials. The reaction mixture was then diluted with dichloromethane and washed with saturated aqueous potassium bicarbonate followed by aqueous hydrochloric acid (1M). The organic layer was then dried over anhydrous magnesium sulfate, filtered and concentrated in vacuo to give an off-white solid. Purification was not necessary; d_{H} (400 MHz, CDCl₃) 8.42 (1H, s, Het-H), 7.92 (1H, dd, ArH), 7.88 (1H, d, ArH), 7.71 (1H, dd, ArH), 7.12 (1H, d, ArH), 6.69 (1H, d, ArH), 4.12 (1H, br m), 3.95-2.95 (15H, m) and 3.06 (3H, s, SO₂Me);

LC/MS (Formic APCI) Found 499 (M+1, T_{R} 1.69 min)

### EXAMPLE 6

### [4-(4-tert-Butyl-phenyl)-piperazin-1-yl]-(5-methanesulfonyl-2-morpholin-4-yl-phenyl)-methanone

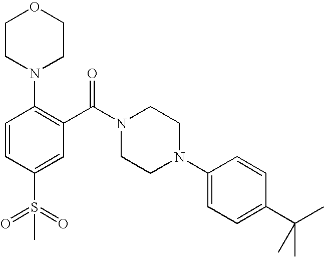

Reaction of 5-methanesulfonyl-2-morpholin-4-yl-benzoic acid and 1-[4-t-butylphenyl]piperazine by the procedure described in Example 5 provided the title compound as a cream solid (20%), d_{H} (400 MHz, DMSO) 7.94 (1H, dd, ArH), 7.74 (1H, d, ArH), 7.31 (3H, m, ArH), 7.02 (2H, br d, ArH), 4.00-3.00 (16H, br m's), 3.20 (3H, s, SO₂Me) and 1.30 (9H, m).

LC/MS (Ammonium bicarbonate APCI) Found 486 (M+1, T_{R} 2.31 min)

### EXAMPLE 7

### [4-(2-Fluoro-4-nitro-phenyl)-piperazin-1-yl]-(5-methanesulfonyl-2-morpholin-4-yl-phenyl)-methanone

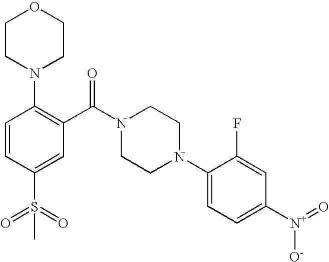

Reaction of 5-methanesulfonyl-2-morpholin-4-yl-benzoic acid and 1-(2-fluoro-4-nitrophenyl)piperazine by the procedure described in Example 5 provided the title compound as an orange solid (75%).

d_{H} (400 MHz, CDCl₃) 8.03 (1H, ddd, ArH), 7.96 (1H, d, ArH), 7.92 (1H, m, ArH), 7.85 (1H, d, ArH), 7.10 (1H, d, ArH), 6.92 (1H, t, ArH), 4.25 (1H, br m), 3.80 (4H, m), 3.68 (1H, ddd), 3.45 (2H, m), 3.35 (4H, m), 3.25 (1H, ddd), 3.05 (3H, s, SO₂Me) and 3.10-2.95 (3H, m).

LC/MS (Ammonium bicarbonate ESI) Found 493 (M+1, T_{R} 1.88 min)

### EXAMPLE 8

### [4-(5-Bromo-pyrimidin-2-yl)-piperazin-1-yl]-(5-methanesulfonyl-2-morpholin-4-yl-phenyl)-methanone

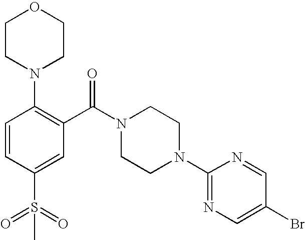

Reaction of 5-methanesulfonyl-2-morpholin-4-yl-benzoic acid and 5-bromo-2-(1-piperazinyl)pyrimidine by the procedure described in Example 5 provided the title compound as an off-white solid (51%), d_{H} (400 MHz, CDCl₃) 8.34 (2H, s), 7.92 (1H, dd, ArH), 7.85 (1H, d, ArH), 7.10 (1H, d, ArH), 4.08 (3H, br m), 3.80-3.65 (6H, m), 3.58 (1H, ddd), 3.35 (3H, m), 3.20 (1H, m), 3.05 (3H, s, SO₂Me) and 3.00 (2H, m).

LC/MS (Ammonium bicarbonate ESI) Found 510/512 (M+1, T_{R} 1.85 min)

### EXAMPLE 9

### 4-[4-(5-Methanesulfonyl-2-morpholin-4-yl-benzoyl)-piperazin-1-yl]-benzonitrile

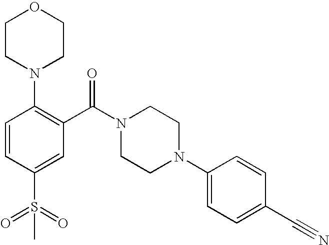

Reaction of 5-methanesulfonyl-2-morpholin-4-yl-benzoic acid and 4-(1-piperazinyl)benzonitrile by the procedure described in Example 5 provided the title compound as a cream solid (83%), d_{H} (400 MHz, CDCl₃) 7.93 (1H, dd, ArH), 7.85 (1H, d, ArH), 7.53 (2H, d, ArH), 7.11 (1H, d, ArH), 6.87 (2H, d, ArH), 4.18 (1H, ddd), 3.80-3.70 (5H, br m), 3.60-3.30 (7H, br m), 3.18 (1H, m), 3.05 (3H, s, SO₂Me) and 3.04-2.90 (2H, m).

LC/MS (Ammonium bicarbonate ESI) Found 455 (M+1, T_{R} 1.64 min)

### EXAMPLE 10

### [4-(2,4-Dichloro-phenyl)-piperazin-1-yl]-(5-methanesulfonyl-2-morpholin-4-yl-phenyl)-methanone

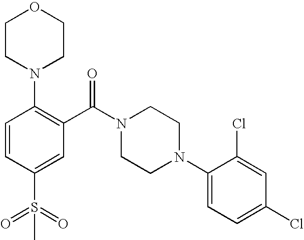

Reaction of 5-methanesulfonyl-2-morpholin-4-yl-benzoic acid and 1-(2,4-dichlorophenyl)piperazine by the procedure described in Example 5 provided the title compound as a white solid (58%), d_{H} (400 MHz, CDCl₃) 7.91 (1H, dd, ArH), 7.85 (1H, d, ArH), 7.39 (1H, d, ArH), 7.22 (1H, dd, ArH), 7.10 (1H, d, ArH), 6.93 (1H, dd, ArH), 4.18 (1H, br m), 3.82 (4H, m), 3.67 (1H, ddd), 3.45 (1H, ddd), 3.42-3.30 (3H, m), 3.22 (1H, m), 3.05 (3H, s, SO₂Me), 3.05-2.90 (4H, m) and 2.85 (1H, ddd).

LC/MS (Ammonium bicarbonate APCI) Found 498/500 (M+1, T_{R} 2.11 min)

### EXAMPLE 11

### [4-(3,5-Dichloro-phenyl)-piperazin-1-yl]-(5-methanesulfonyl-2-morpholin-4-yl-phenyl)-methanone

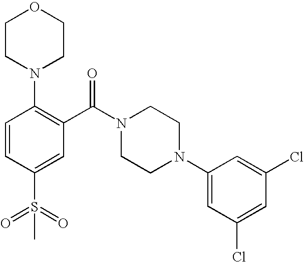

Reaction of 5-methanesulfonyl-2-morpholin-4-yl-benzoic acid and the substituted piperazine by the procedure described in Example 5 provided the title compound isolated as a white solid (52%).

d_{H} (400 MHz, CDCl₃) 7.92 (1H, dd, ArH), 7.85 (1H, d, ArH), 7.10 (1H, d, ArH), 6.87 (1H, t, ArH), 6.74 (2H, d, ArH), 4.11 (1H, dddd), 3.87-3.70 (5H, m), 3.50-3.20 (7H, m), 3.05 (3H, s, SO₂Me) and 3.05-2.90 (3H, m).

LC/MS (Ammonium bicarbonate ESI) Found 498/500 (M+1, T_{R} 2.24 min)

### EXAMPLE 12

### [4-(3,4-Dimethyl-phenyl)-piperazin-1-yl]-(5-methanesulfonyl-2-morpholin-4-yl-phenyl)-methanone

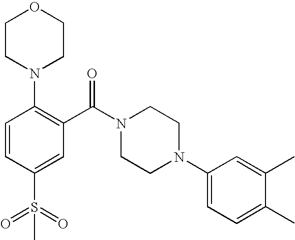

Reaction of 5-methanesulfonyl-2-morpholin-4-yl-benzoic acid and the substituted piperazine by the procedure described in Example 5 provided the title compound isolated as an off-white solid (44%).

d_{H} (400 MHz, CDCl₃) 7.91 (1H, dd, ArH), 7.85 (1H, d, ArH), 7.08 (1H, d, ArH), 7.04 (1H, d, ArH), 6.74 (1H, br d, ArH), 6.68 (1H, dd, ArH), 4.13 (1H, m), 3.80 (1H, m), 3.80 (5H, m), 3.40 (3H, m), 3.28 (2H, m), 3.15 (2H, m), 3.04 (3H, s, SO₂Me), 3.03-2.90 (2H, m), 2.24 (3H, s, Me) and 2.19 (3H, s, Me).

LC/MS (Ammonium bicarbonate ESI) Found 458 (M+1, T_{R} 1.97 min)

### EXAMPLE 13

### [4-(4-Chloro-3-trifluoromethyl-phenyl)-piperazin-1-yl]-(5-methanesulfonyl-2-morpholin-4-yl-phenyl)-methanone

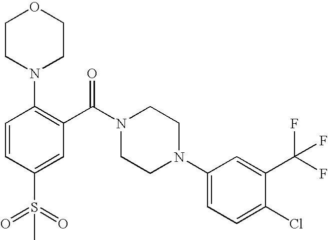

Reaction of 5-methanesulfonyl-2-morpholin-4-yl-benzoic acid and the substituted piperazine by the procedure described in Example 5 provided the title compound isolated as a white solid (65%).

d_{H} (400 MHz, CDCl₃) 7.92 (1H, dd, ArH), 7.85 (1H, d, ArH), 7.38 (1H, d, ArH), 7.18 (1H, d, ArH), 7.10 (1H, d, ArH), 6.97 (1H, dd, ArH), 4.20 (1H, m), 3.90-3.70 (5H, m), 3.50-3.20 (7H, m), 3.05 (3H, s, SO₂Me) and 3.05-2.90 (3H, m).

LC/MS (Ammonium bicarbonate ESI) Found 532/534 (M+1, T_{R} 2.20 min)

### EXAMPLE 14

### [4-(2,4-Difluoro-phenyl)-piperazin-1-yl]-(5-methanesulfonyl-2-morpholin-4-yl-phenyl)-methanone

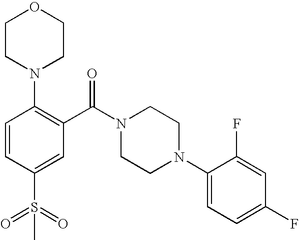

Reaction of 5-methanesulfonyl-2-morpholin-4-yl-benzoic acid and the substituted piperazine by the procedure described in Example 5 provided the title compound isolated as a white solid (65%).

d_{H} (400 MHz, CDCl₃) 7.91 (1H, dd, ArH), 7.85 (1H, d, ArH), 7.08 (1H, d, ArH), 6.92-6.79 (3H, m, ArH), 4.25 (1H, br ddd), 3.82 (4H, m), 3.68 (1H, ddd), 3.45 (1H, ddd), 3.37 (2H, m), 3.30 (1H, m), 3.21 (1H, m), 3.08 (3H, s, SO₂Me), 3.10-2.95 (4H, m) and 2.83 (1H, ddd).

LC/MS (Ammonium bicarbonate ESI) Found 466 (M+1, T_{R} 1.87 min).

### EXAMPLE 15

### [4-(3,5-Bis-trifluoromethyl-phenyl)-piperazin-1-yl]-(5-methanesulfonyl-2-morpholin-4-yl-phenyl)-methanone

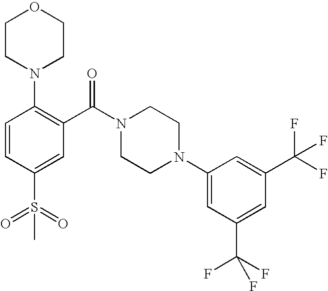

Reaction of 5-methanesulfonyl-2-morpholin-4-yl-benzoic acid and the substituted piperazine by the procedure described in Example 5 provided the title compound isolated as a white solid (91%).

Aliphatics complex: d_{H} (400 MHz, CDCl₃) 7.93 (1H, dd, ArH), 7.85 (1H, d, ArH), 7.36 (1H, s, ArH), 7.25 (2H, s, ArH), 7.11 (1H, d, ArH), 4.23 (1H, m), 3.80 (5H, m), 3.50 (2H, m), 3.35 (5H, m), 3.05 (3H, s, SO₂Me) and 3.20-2.95 (3H, m).

LC/MS (Ammonium bicarbonate ESI) Found 566 (M+1, T_{R} 2.31 min)

### EXAMPLE 16

### [4-(3-Bromo-phenyl)-piperazin-1-yl]-(5-methanesulfonyl-2-morpholin-4-yl-phenyl)-methanone

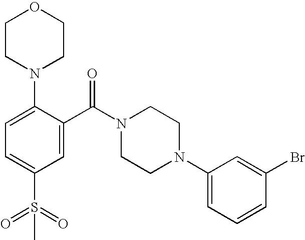

Reaction of 5-methanesulfonyl-2-morpholin-4-yl-benzoic acid and the substituted piperazine by the procedure described in Example 5 provided the title compound isolated as a white solid (52%).

d_{H} (400 MHz, CDCl₃) 7.91 (1H, dd, ArH), 7.84 (1H, d, ArH), 7.15 (1H, t, ArH), 7.10 (1H, d, ArH), 7.03 (2H, m, ArH), 6.82 (1H, dd, ArH), 4.13 (1H, ddd), 3.80 (5H, m), 3.48-3.15 (7H, m), 3.05 (3H, s, SO₂Me) and 3.05-2.95 (3H, m).

LC/MS (Ammonium bicarbonate APCI) Found 508/510 (M+1, T_{R} 2.01 min).

### EXAMPLE 17

### 2-[4-(5-Methanesulfonyl-2-morpholin-4-yl-benzoyl)-piperazin-1-yl]-benzonitrile

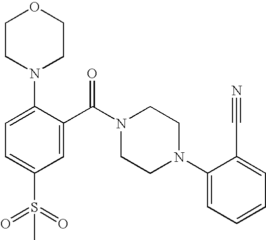

Reaction of 5-methanesulfonyl-2-morpholin-4-yl-benzoic acid and the substituted piperazine by the procedure described in Example 5 provided the title compound isolated as a white solid (73%).

d_{H} (400 MHz, CDCl₃) 7.91 (1H, dd, ArH), 7.86 (1H, d, ArH), 7.60 (1H, dd, ArH), 7.51 (1H, td, ArH), 7.10 (1H, td, ArH), 7.08 (1H, d, ArH), 7.00 (1H, d, ArH), 4.41 (1H, ddd), 3.80 (4H, m), 3.63 (1H, ddd), 3.55 (1H, ddd), 3.45-3.20 (5H, m), 3.12 (1H, ddd), 3.04 (3H, s, SO₂Me) and 3.05-2.90 (3H, m).

LC/MS (Ammonium bicarbonate ESI) Found 455 (M+1, T_{R} 1.71 min)

### EXAMPLE 18

### [4-(4-Chloro-2-fluoro-phenyl)-piperazin-1-yl]-(5-methanesulfonyl-2-morpholin-4-yl-phenyl)-methanone

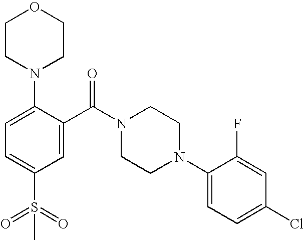

Reaction of 5-methanesulfonyl-2-morpholin-4-yl-benzoic acid and the substituted piperazine by the procedure described in Example 5 provided the title compound isolated as a white solid (61%).

d_{H} (400 MHz, CDCl₃) 7.91 (1H, dd, ArH), 7.86 (1H, d, ArH), 7.09 (3H, m, ArH), 6.84 (1H, t, ArH), 4.20 (1H, ddd), 3.80 (4H, m), 3.68 (1H, ddd), 3.45 (1H, ddd), 3.40-3.15 (4H, m), 3.05 (3H, s, SO₂Me), 3.11-2.95 (4H, m) and 2.86 (1H, ddd).

LC/MS (Ammonium bicarbonate ESI) Found 482/484 (M+1, T_{R} 2.03 min)

### EXAMPLE 19

### [4-(3-Fluoro-phenyl)-piperazin-1-yl]-(5-methanesulfonyl-2-morpholin-4-yl-phenyl)-methanone

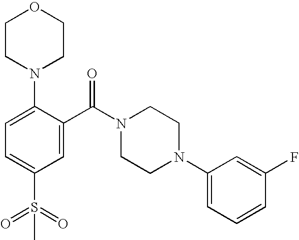

Reaction of 5-methanesulfonyl-2-morpholin-4-yl-benzoic acid and the substituted piperazine by the procedure described in Example 5 provided the title compound isolated as a white solid (51%).

d_{H} (400 MHz, CDCl₃) 7.92 (1H, dd, ArH), 7.84 (1H, d, ArH), 7.22 (1H, dt, ArH), 7.10 (1H, d, ArH), 6.66 (1H, dd, ArH), 6.60 (2H, m, ArH), 4.13 (1H, ddd), 3.78 (5H, m), 3.48-3.15 (7H, m), 3.05 (3H, s, SO₂Me) and 3.05-2.95 (3H, m).

LC/MS (Ammonium bicarbonate ESI) Found 448 (M+1, T_{R} 1.83 min).

### EXAMPLE 20

### [4-(3-Chloro-4-methoxy-phenyl)-piperazin-1-yl]-(5-methanesulfonyl-2-morpholin-4-yl-phenyl)-methanone

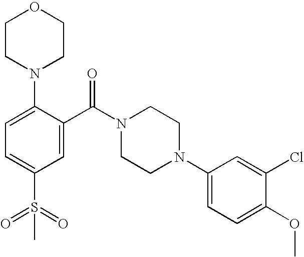

Reaction of 5-methanesulfonyl-2-morpholin-4-yl-benzoic acid and the substituted piperazine by the procedure described in Example 5 provided the title compound isolated as an off-white solid (63%).

Aliphatics complex: d_{H} (400 MHz, CDCl₃) 7.92 (1H, dd, ArH), 7.84 (1H, d, ArH), 7.09 (1H, d, ArH), 6.99 (1H, d, ArH), 6.87 (1H, d, ArH), 6.80 (1H, dd, ArH), 4.15 (1H, ddd), 3.86 (3H, s, OMe), 3.80-3.70 (5H, m), 3.48-3.15 (6H, m), 3.03 (3H, s, SO₂Me), 3.15-2.95 (3H, m) and 2.95 (1H, ddd).

LC/MS (Ammonium bicarbonate ESI) Found 494 (M+1, T_{R} 1.81 min).

### EXAMPLE 21

### [4-(3-Chloro-4-fluoro-phenyl)-piperazin-1-yl]-(5-methanesulfonyl-2-morpholin-4-yl-phenyl)-methanone

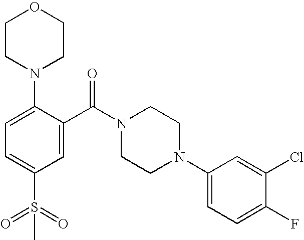

Reaction of 5-methanesulfonyl-2-morpholin-4-yl-benzoic acid and the substituted piperazine by the procedure described in Example 5 provided the title compound isolated as a white solid (27%).

d_{H} (400 MHz, CDCl₃) 7.91 (1H, dd, ArH), 7.84 (1H, d, ArH), 7.09 (1H, d, ArH), 7.03 (1H, t, ArH), 6.92 (1H, dd, ArH), 6.76 (1H, td, ArH), 4.16 (1H, ddd), 3.81 (5H, m), 3.38-3.25 (5H, m), 3.15 (2H, m), 3.05 (3H, s, SO₂Me), 3.04-2.95 (2H, m) and 2.92 (1H, ddd).

LC/MS (Ammonium bicarbonate APCI) Found 482 (M+1, T_{R} 1.87 min)

### EXAMPLE 22

### 5-[4-(5-Methanesulfonyl-2-morpholin-4-yl-benzoyl)-piperazin-1-yl]-2-methoxy-benzonitrile

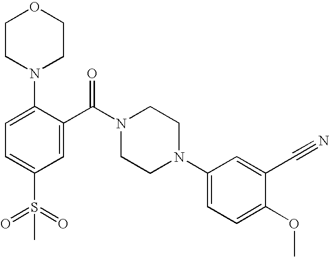

Reaction of 5-methanesulfonyl-2-morpholin-4-yl-benzoic acid and the substituted piperazine by the procedure described in Example 5 provided the title compound isolated as an off-white solid.

d_{H} (400 MHz, CDCl₃) 7.92 (1H, dd, ArH), 7.84 (1H, d, ArH), 7.09 (1H, d, ArH), 6.99 (1H, d, ArH), 6.87 (1H, d, ArH), 6.80 (1H, dd, ArH), 4.19 (1H, ddd), 3.89 (3H, s, OMe), 3.85-3.68 (5H, m), 3.50-3.15 (6H, m), 3.05 (3H, s, SO₂Me), 3.15-2.95 (3H, m) and 2.92 (1H, ddd).

LC/MS (Ammonium bicarbonate APCI) Found 485 (M+1, T_{R} 1.81 min)

### EXAMPLE 23

### (5-Methanesulfonyl-2-morpholin-4-yl-phenyl)-[4-(4-methanesulfonyl-phenyl)-piperazin-1-yl]-methanone

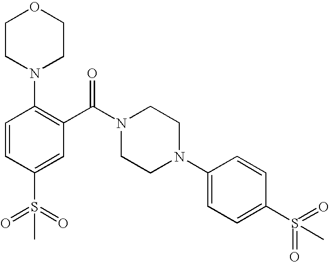

Reaction of 5-methanesulfonyl-2-morpholin-4-yl-benzoic acid and the substituted piperazine by the procedure described in Example 5 provided the title compound isolated as a yellow solid (49%).

d_{H} (400 MHz, CDCl₃) 7.91 (1H, dd, ArH), 7.83 (1H, d, ArH), 7.79 (2H, d, ArH), 7.12 (1H, d, ArH), 6.97 (2H, d, ArH), 4.01-3.08 (16H, bm), 3.05 (3H, s, SO₂Me), 3.01 (3H, s, SO₂Me)

LC/MS (Ammonium bicarbonate ESI) Found 508 (M+1, T_{R} 1.41 min)

### EXAMPLE 24

### [4-(3,4-Difluoro-phenyl)-piperazin-1-yl]-(5-methanesulfonyl-2-morpholin-4-yl-phenyl)-methanone

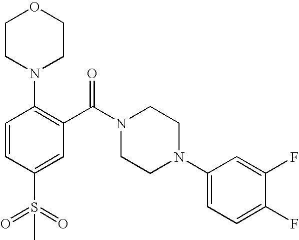

Reaction of 5-methanesulfonyl-2-morpholin-4-yl-benzoic acid and the substituted piperazine by the procedure described in Example 5 provided the title compound isolated as a yellow solid (66%).

d_{H} (400 MHz, CDCl₃) 7.92 (1H, dd, ArH), 7.84 (1H, d, ArH), 7.08 (1H, d, ArH), 7.03 (1H, t, ArH), 6.72 (1H, dd, ArH), 6.60 (1H, m, ArH), 4.16 (1H, m), 3.81 (5H, m), 3.48-3.25 (5H, m), 3.12 (2H, m), 3.05 (3H, s, SO₂Me), 3.04-2.95 (2H, m) and 2.92 (1H, ddd).

LC/MS (Ammonium bicarbonate ESI) Found 466 (M+1, T_{R} 1.75 min)

### EXAMPLE 25

### [4-(3,5-Difluoro-4-methoxy-phenyl)-piperazin-1-yl]-(5-methanesulfonyl-2-morpholin-4-yl-phenyl)-methanone

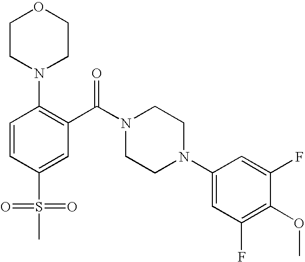

Reaction of 5-methanesulfonyl-2-morpholin-4-yl-benzoic acid and the substituted piperazine by the procedure described in Example 5 provided the title compound, d_{H} (400 MHz, CDCl₃) 7.92 (1H, dd), 7.84 (1H, d), 7.18 (1H, d), 6.42 (2H, d), 4.16 (1H, m), 3.92 (3H, s), 3.80 (5H, m), 3.40 (3H, m), 3.28 (2H, m), 3.13 (2H, m), 3.05 (3H, s), 3.02 (2H, m), 2.92 (1H, ddd)

LC/MS (Ammonium bicarbonate APCI) Found 496 (M+1, T_{R} 1.85 min)

### EXAMPLE 26

### 4-[4-(5-Methanesulfonyl-2-piperidin-1-yl-benzoyl)-piperazin-1-yl]-benzonitrile

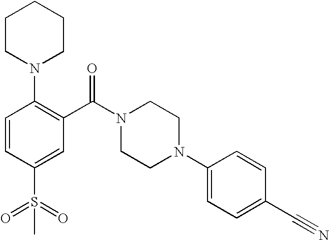

Reaction of 5-methanesulfonyl-2-(1-piperidinyl)benzoic acid and the substituted piperazine by the procedure described in Example 5 provided the title compound, isolated as an off-white solid.

d_{H} (400 MHz, CDCl₃) 7.87 (1H, dd, ArH), 7.84 (1H, d, ArH), 7.55 (2H, d, ArH), 7.06 (1H, d, ArH), 6.85 (2H, d, ArH), 4.18 (1H, br m), 3.75 (1H, m), 3.65-3.25 (7H, m), 3.15 (1H, m), 3.05 (3H, s, SO₂Me), 3.00 (2H, m) and 1.65 (6H, m).

LC/MS (Ammonium bicarbonate APCI) Found 453 (M+1, T_{R} 2.02 min).

### EXAMPLE 27

### 4-(3,5-Difluoro-4-methoxy-phenyl)-piperazin-1-yl]-(5-methanesulfonyl-2-piperidin-1-yl-phenyl)-methanone

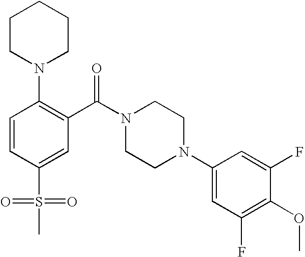

Reaction of 5-methanesulfonyl-2-(1-piperidinyl)benzoic acid and the substituted piperazine by the procedure described in Example 5 provided the title compound,

d_{H} (400 MHz, CDCl₃) 7.86 (1H, dd), 7.81 (1H, d), 7.05 (1H, d), 6.43 (2H, d), 4.15 (1H, m), 3.90 (3H, s. OMe), 3.70 (1H, ddd), 3.45 (1H, ddd), 3.25 (4H, m), 3.10 (2H, m), 3.03 (3H, s), 3.01 (2H, m), 2.90 (1H, ddd), 1.65 (6H, m)

LC/MS (Ammonium bicarbonate APCI) Found 494 (M+1, T_{R} 2.21 min)

### EXAMPLE 28

### [4-(4-Chloro-3-nitro-phenyl)-piperazin-1-yl]-(5-methanesulfonyl-2-piperidin-1-yl-phenyl)-methanone

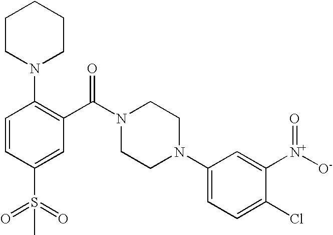

A reaction mixture of 5-methanesulfonyl-2-piperidin-1-yl-benzoic acid (103 mg, 0.36 mmol), HBTU (150 mg, 0.40 mmol) and diisopropylamine (0.26 ml, 1.5 mmol) in dry tetrahydrofuran (10 ml) and dry N,N-dimethylformamide (1.2 ml) was stirred for 15 minutes at room temperature under nitrogen atmosphere. 1-(4-Chloro-3-nitro-phenyl)-piperazine hydrochloride (117 mg, 0.42 mmol) was then added and the resulting mixture was stirred at room temperature for 2 days. The reaction mixture was concentrated in vacuo and water was added to the residue obtained. The mixture was left stirring for 10 minutes and the resulting precipitate was then extracted with ethyl acetate. The organic layer was washed with saturated brine, dried over anhydrous magnesium sulfate, filtered and concentrated in vacuo to give the crude product. Purification by flash chromatography (SiO₂) afforded the desired product as an orange solid.

d_{H} (400 MHz, CDCl₃) 7.88 (1H, dd, ArH), 7.84 (1H, d, ArH), 7.40 (1H, d, ArH), 7.34 (1H, d, ArH), 7.07 (1H, d, ArH), 7.00 (1H, dd, ArH), 4.20 (1H, br m), 3.74 (1H, ddd), 3.42 (2H, m), 3.26 (5H, m), 3.04 (3H, s, SO₂Me), 3.00 (3H, m) and 1.69-1.59 (6H, m);

LC/MS (Ammonium bicarbonate APCI) Found 507/509 (M+1, T_{R} 2.34 min)

### EXAMPLE 29

### [4-(3,5-Dichloro-4-methoxy-phenyl)-piperazin-1-yl]-(5-methanesulfonyl-2-piperidin-1-yl-phenyl)-methanone

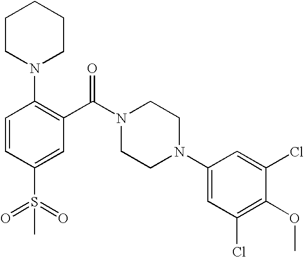

A reaction mixture of 5-methanesulfonyl-2-piperidin-1-yl-benzoic acid (70 mg, 0.25 mmol), HBTU (95 mg, 0.25 mmol) and diisopropylamine (0.16 ml, 0.92 mmol) in dry tetrahydrofuran (10 ml) and dry N,N-dimethylformamide (1.2 ml) was stirred for 30 minutes at room temperature under nitrogen atmosphere. 1-(3,5-Dichloro-4-methoxy-phenyl)-piperazine hydrochloride (80 mg, 0.31 mmol) was then added and the resulting mixture was stirred at room temperature for 18 hrs. The reaction mixture was concentrated in vacuo and water was added to the residue obtained. The mixture was left stirring for 10 minutes and the resulting precipitate was then extracted with ethyl acetate. The organic layer was washed with saturated brine, dried over anhydrous magnesium sulfate, filtered and concentrated in vacuo to give the crude product. Purification by flash chromatography (SiO₂) afforded the desired product as a white solid.

d_{H} (400 MHz, CDCl₃) 7.87 (1H, dd, ArH), 7.82 (1H, d, ArH), 7.07 (1H, d, ArH), 6.82 (2H, s, 2×ArH), 4.16 (1H, br m), 3.84 (3H, s, OMe), 3.73 (1H, br m), 3.42 (1H, br m), 3.37-2.85 (9H, m), 3.04 (3H, s, SO₂Me) and 1.69-1.60 (6H, m).

LC/MS (Ammonium bicarbonate ESI/APCI) Found 526/528 (M+1, T_{R} 2.50 min).

### EXAMPLE 30

### [4-(3,4-Dichloro-phenyl)-piperazin-1-yl]-(5-methanesulfonyl-2-piperidin-1-yl-phenyl)-methanone

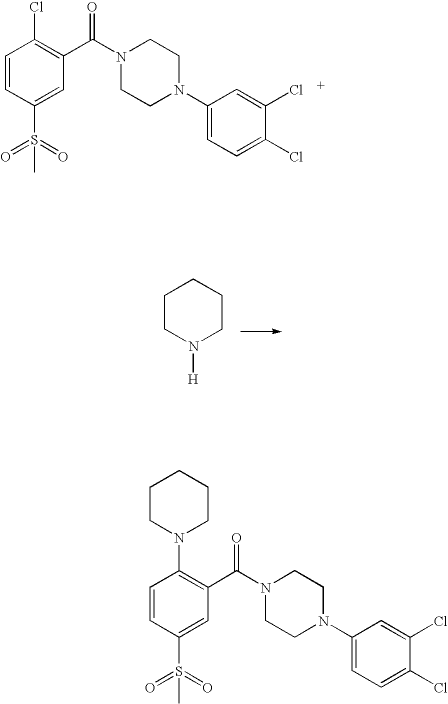

A reaction mixture of piperidine (2 equiv. or more) and (2-chloro-5-methanesulfonyl-phenyl)-[4-(3,4-dichloro-phenyl)-piperazin-1-yl]-methanone (1 equiv.) was heated (with or without diisopropylamine as solvent) at 180° C. on the CEM Discover® microwave for 10 minutes. The resulting residue was then dissolved in dichloromethane (ethyl acetate, if diisopropylamine was used). The organic solution was washed with saturated brine, dried over anhydrous magnesium sulfate, filtered and concentrated in vacuo to give the crude product. Purification by flash chromatography gave the pure title compound, isolated as a colourless oil.

d_{H} (400 MHz, CDCl₃) 7.87 (1H, dd, ArH), 7.82 (1H, d, ArH), 7.30 (1H, d, ArH), 7.06 (1H, d, ArH), 6.95 (1H, d, ArH), 6.74 (1H, dd, ArH), 4.15 (1H, br m), 3.72 (1H, br m), 3.46-2.92 (10H, m), 3.04 (3H, s) and 1.69-1.59 (6H, m).

LC/MS (Ammonium bicarbonate APCI) Found 496/498 (M+1, T_{R} 2.53 min)

### EXAMPLE 31

### [4-(3,4-Dichloro-phenyl)-piperazin-1-yl]-(5-methanesulfonyl-2-propylamino-phenyl)-methanone

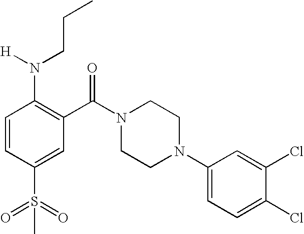

Reaction of 5-(methylsulfonyl)-2-(propylamino)benzoic acid and 1-(3,4-dichlorophenyl)piperazine by the procedure described in Example 5 provided the title compound, isolated as a white solid.

d_{H} (400 MHz, CDCl₃) 7.76 (1H, dd, ArH), 7.65 (1H, d, ArH), 7.31 (1H, d, ArH), 6.97 (1H, d, ArH), 6.75 (2H, 2×d, ArH), 6.00 (1H, t, NH), 3.77 (4H, br s), 3.18 (4H br t), 3.14 (2H, dt), 2.99 (3H, s, SO₂Me), 1.67 (2H, hex) and 1.01 (3H, t).

LC/MS (Formic APCI) Found 471/473 (M+1, T_{R} 2.19 min)

### EXAMPLE 32

### [4-(3,4-Dichloro-phenyl)-piperazin-1-yl]-[2-(4,4-dimethyl-piperidin-1-yl)-5-methanesulfonyl-phenyl]-methanone

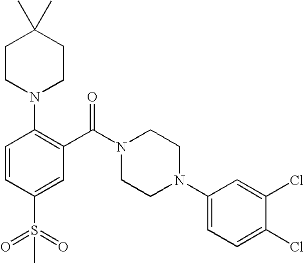

Reaction of 2-(4,4-dimethyl-1-piperidinyl)-5-(methylsulfonyl)benzoic acid and 1-(3,4-dichlorophenyl)piperazine by the procedure described in Example 5 provided the title compound, isolated as a white solid.

d_{H} (400 MHz, CDCl₃) 7.85 (1H, dd, ArH), 7.81 (1H, d, ArH), 7.30 (1H, d, ArH), 7.09 (1H, d, ArH), 6.97 (1H, d, ArH), 6.74 (1H, dd, ArH), 4.15 (1H, br m), 3.72 (1H, m), 3.40 (1H, m), 3.29 (4H, m), 3.19 (2H, m), 3.10-2.95 (3H, m), 3.01 (3H, s, SO₂Me), 1.45 (4H, m) and 0.98 (6H, s).

LC/MS (Ammonium bicarbonate ESI) Found 524/526 (M+1, T_{R} 2.11 min)

### EXAMPLE 33

### [4-(3,4-Dichloro-phenyl)-piperazin-1-yl]-[5-methanesulfonyl-2-(methyl-propyl-amino)-phenyl]-methanone

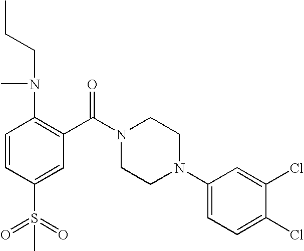

Reaction of 2-[methyl(propyl)amino]-5-(methylsulfonyl)benzoic acid and 1-(3,4-dichlorophenyl)piperazine by the procedure described in Example 5 provided the title compound, isolated as a white solid.

d_{H} (400 MHz, CDCl₃) 7.76 (1H, dd, ArH), 7.71 (1H, d, ArH), 7.30 (1H, d, ArH), 6.97 (1H, d, ArH), 6.91 (1H, d, ArH), 6.74 (1H, dd, ArH), 6.00 (1H, t, NH), 4.10 (1H, m), 3.77 (1H, br s), 3.45 (2H, m), 3.30-3.00 (6H, m), 3.04 (3H, s, SO₂Me), 2.95 (3H, s, NMe), 1.63 (1H) and 0.89 (3H, t).

LC/MS (Ammonium bicarbonate ESI) Found 484/486 (M+1, T_{R} 2.46 min)

### EXAMPLE 34

### [4-(3,4-Dichloro-phenyl)-piperazin-1-yl]-[5-methanesulfonyl-2-(2-methyl-piperidin-1-yl)-phenyl]-methanone

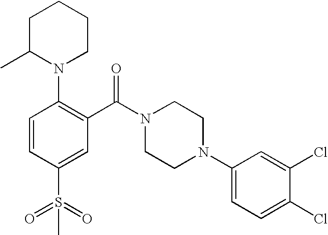

Reaction of 2-(2-methyl-1-piperidinyl)-5-(methylsulfonyl)benzoic acid and 1-(3,4-dichlorophenyl)piperazine by the procedure described in Example 5 provided the title compound, isolated as an off-white solid.

d_{H} (400 MHz, CDCl₃) [rotamers present in 3:2 ratio] 7.91-7.83 (4H, m, ArH), 7.29 (2H, d, ArH), 7.06 (2H, d, ArH), 6.96 (2H, d, ArH), 6.73 (2H, dd, ArH), 4.3-3.51 (5H, m), 3.50-3.10 (13H, m), 3.10-3.00 (6H, m, SO2Me), 3.00-2.68 (4H, m), 1.57-1.48 (10H, m), 1.34-1.15 (2H, m), 0.98 (3H, d, Me), 0.87 (3H, d, Me).

LC/MS (Ammonium bicarbonate ESI) Found 510/512 (M+1, T_{R} 2.66 min).

### EXAMPLE 35

### 1-[3-Fluoro-4-(4-{[5-(methylsulfonyl)-2-(1-piperidinyl)phenyl]carbonyl}-1-piperazinyl)phenyl]ethanone

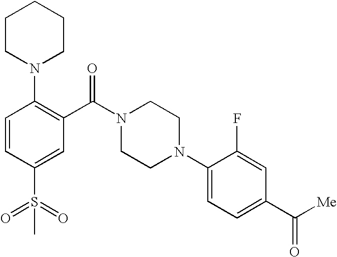

5-Methanesulfonyl-2-piperidin-1-yl-benzoic acid (0.500 g) and 1-[3-fluoro-4-(1-piperazinyl)phenyl]ethanone (0.393 g) were dissolved in MDC (50 ml) and stirred at room temperature for 16 hours, under an argon atmosphere, with EDC (0.694 g) and triethylamine (0.294 ml). The reaction solution was then washed with saturated sodium hydrogen carbonate solution and saturated sodium chloride solution. The organic solution was dried over anhydrous magnesium sulphate, filtered and concentrated at reduced pressure to yield the crude product, which was purified by silica gel column chromatography. Elution with a gradient of 0 to 100% ethyl acetate in pentane provided the title compound as a white foam (66% yield), LC/MS (ESI) Found 488 (M+1), 510 (M+23).

### EXAMPLE 36

### 1-[3-Fluoro-4-(4-{[5-(methylsulfonyl)-2-(1-piperidinyl)phenyl]carbonyl}-1-piperazinyl)phenyl]ethanone O-methyloxime

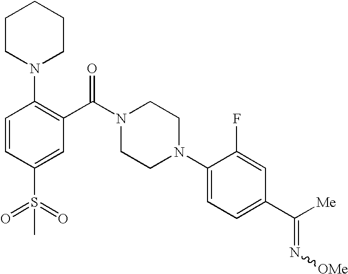

1-[3-Fluoro-4-(4-{[5-(methylsulfonyl)-2-(1-piperidinyl)phenyl]carbonyl}-1-piperazinyl)phenyl]ethanone (0.100 g), hydroxylamine hydrochlide (0.017 g) and methyl iodide (0.048 ml) were dissolved in dimethysulfoxide (5 ml). a solution of potassium hydroxide (0.098 g) in water (2 ml) was added to the stirred solution and stirring was continued at room temperature for 4 h. The reaction solution was evaporated to small volume at reduced pressure and partitioned between dichloromethane and water. The aqueous solution was extracted twice with dichloromethane and the combined organic extracts were dried (MgSO4) and evaporated. The residue was chromatographed over silica gel, eluting with a gradient of 0 to 100% ethyl acetate in pentane. The title compound was obtained as a colourless oil, (52% yield), LC/MS (ESI) Found 517 (M+1).

### EXAMPLE 37

### 1-[3-Fluoro-4-(4-{[5-(methylsulfonyl)-2-(1-piperidinyl)phenyl]carbonyl}-1-piperazinyl)phenyl]ethanol

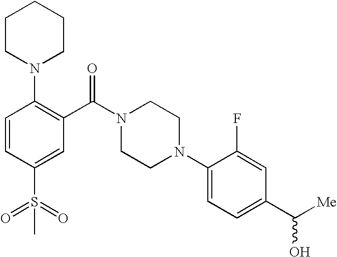

1-[3-Fluoro-4-(4-{[5-(methylsulfonyl)-2-(1-piperidinyl)phenyl]carbonyl}-1-piperazinyl)phenyl]ethanone (0.100 g) was dissolved in methanol (5 ml) and sodium borohydride (8 mg) was added to the stirred solution. Stirring was continued at room temperature for 2 h. The reaction solution was then partitioned between ethyl acetate and water. The organic phase was washed with brine, dried (MgSO4) and evaporated. The residue was chromatographed over silica gel, eluting with a gradient of 0 to 100% ethyl acetate in pentane. The title compound was obtained as a white foam, (99% yield),

LC/MS (ESI) Found 490 (M+1), 512 (M+23).

### EXAMPLE 38

### 1-{2-Fluoro-4-[1-(methyloxy)ethyl]phenyl}-4-{[5-(methylsulfonyl)-2-(1-piperidinyl)phenyl]carbonyl}piperazine

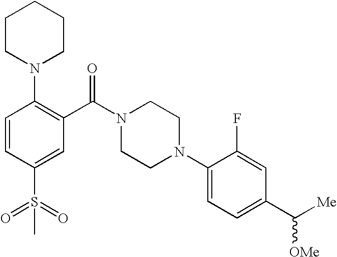

1-[3-Fluoro-4-(4-{[5-(methylsulfonyl)-2-(1-piperidinyl)phenyl]carbonyl}-1-piperazinyl)phenyl]ethanol (0.095 g) was dissolved in N,N-dimethylformamide (5 ml) and stirred at room temperature under argon. Sodium hydride (60% dispersion in oil, 10 mg) was added, followed by methyl iodide (0.016 ml). Stirring was continued at room temperature for 2 h. The reaction mixture was then partitioned between ethyl acetate and water. The organic solution was washed twice with water, dried (MgSO₄) and evaporated to yield the pure product as a white waxy solid (89% yield), LC/MS Found 504 (ESI) (M+1).

### EXAMPLE 39

### {1-[3-Fluoro-4-(4-{[5-(methylsulfonyl)-2-(1-piperidinyl)phenyl]carbonyl}1-piperazinyl)phenyl]ethylidene}propanedinitrile

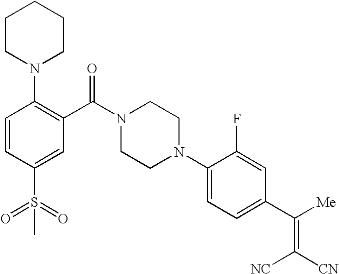

1-[3-Fluoro-4-(4-{[5-(methylsulfonyl)-2-(1-piperidinyl)phenyl]carbonyl}-1-piperazinyl)phenyl]ethanone (100 mg), malononitrile (14 mg) and basic alumina (pH 9-10, Fisher Scientific Beckmann Type II alkaline, 60 mg) were heated to 100° C. for 1 h. After cooling, the residue was chromatographed over silica gel, eluting with a gradient of 0 to 100% ethyl acetate in pentane. The title compound was obtained as a pale yellow foam, (88% yield), LC/MS (ESI) Found 536 (M+1).

### EXAMPLE 40

### 1-(3,4-dichlorophenyl)-4-{[5-(ethylsulfonyl)-2-(1-piperidinyl)phenyl]carbonyl}piperazine

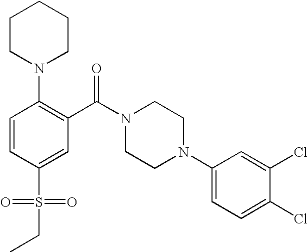

A solution of 5-(ethylsulfonyl)-2-(1-piperidinyl)benzoic acid (50 mg), 1-(3,4-dichlorophenyl)piperazine (39 mg), EDCI (38 mg) and HOBt (10 mg) in DCM (2 ml) was stirred for 18 hours. The reaction mixture was washed with saturated sodium bicarbonate solution and applied to silica gel column. Elution with 20-100% ethyl acetate in pentane gave the desired product as a crisp foam (74 mg, 86%). LC/MS Found 510 (ESI) (M+1).

### EXAMPLE 41

### 1-[3,5-Dichloro-4-(methyloxy)phenyl]-4-{[5-(ethylsulfonyl)-2-(-piperidinyl)phenyl]carbonyl}piperazine

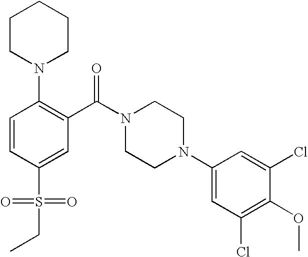

The title compound was prepared from 5-(ethylsulfonyl)-2-(1-piperidinyl)benzoic acid (50 mg) and 1-[3,5-dichloro-4-(methyloxy)phenyl]piperazine HCl salt (51 mg) by method of Example 53. Obtained desired product as crisp foam (86 mg, 93%). LC/MS Found 540 (ESI) (M+1).

### EXAMPLE 42

### 1-(3,4-Dichlorophenyl)-4-{[5-[(1-methylethyl)sulfonyl]-2-(1-piperidinyl)phenyl]carbonyl}piperazine

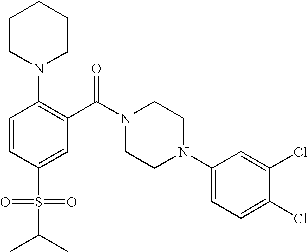

Title compound prepared as described in Example 53. Obtained as crisp foam (84 mg, 100%). LC/MS Found 524 (ESI) (M+1).

### EXAMPLE 43

### 1-[3,5-Dichloro-4-(methyloxy)phenyl]-4-{[5-[(1-methylethyl)sulfonyl]-2-(1-piperidinyl)phenyl]carbonyl}piperazine

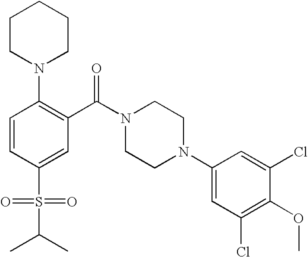

Title compound prepared as described in Example 53. Obtained as crisp foam (89 mg, 100%). LC/MS Found 554 (ESI) (M+1).

### EXAMPLE 44

### 1-[3,5-Dichloro-4-(methyloxy)phenyl]-4-{[5-(methylsulfinyl)-2-(1-piperidinyl)phenyl]carbonyl}piperazine

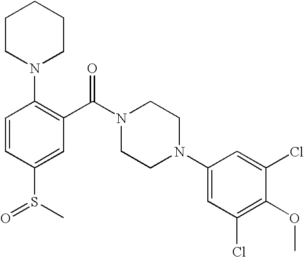

Title compound prepared as described in Example 53. Obtained as crisp foam (30 mg, 60%). LC/MS Found 532 (ESI) (M+Na).

The following compounds were prepared according to the procedure of either Example 35 or Example 58.

| Example | | | LC/MS |
|---|---|---|---|
| No. | Structure | Name | (ESI) |
| 45 | 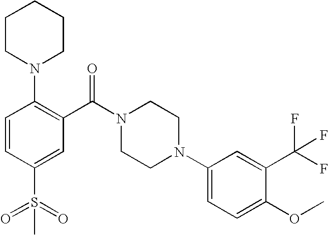 | 1-[4-(methyloxy)-3-(trifluoromethyl)phenyl]- 4-{[5-(methylsulfonyl)-2-(1- piperidinyl)phenyl]carbonyl}piperazine | |
| 46 | 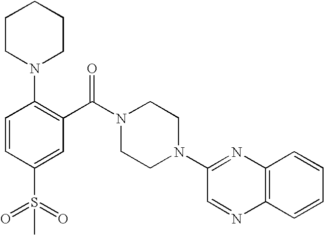 | 2-(4-{[5-(methylsulfonyl)-2-(1- piperidinyl)phenyl]carbonyl}-1- piperazinyl)quinoxaline | 480 (M + H) |
| 47 | 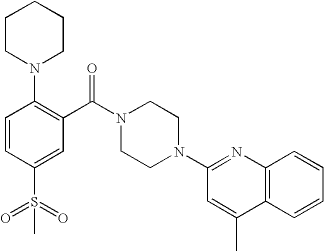 | 4-methyl-2-(4-{[5-(methylsulfonyl)-2-(1- piperidinyl)phenyl]carbonyl}-1- piperazinyl)quinoline | 493 (M + H) |
| 48 | 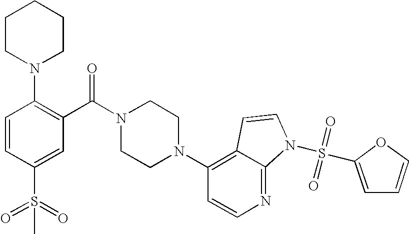 | 1-(2-furanylsulfonyl)-4-(4-{[5- (methylsulfonyl)-2-(1- piperidinyl)phenyl]carbonyl}-1-piperazinyl)- 1H-pyrrolo[2,3-b]pyridine | 598 (M + H) |
| 49 | 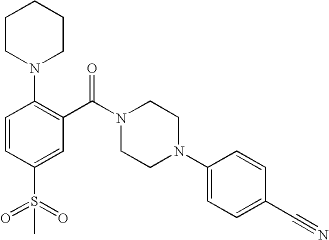 | 4-(1-{[5-(methylsulfonyl)-2-(1- piperidinyl)phenyl]carbonyl}-4- piperazinyl)benzonitrile | 474 (M + Na) |
| 50 | 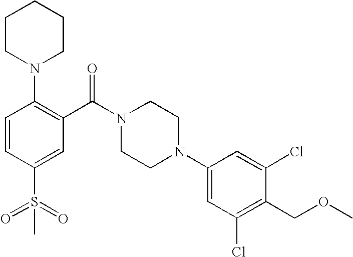 | 1-{3,5-dichloro-4- [(methyloxy)methyl]phenyl}-4-{[5- (methylsulfonyl)-2-(1- piperidinyl)phenyl]carbonyl}piperazine | 540 (M + H) |
| 51 | 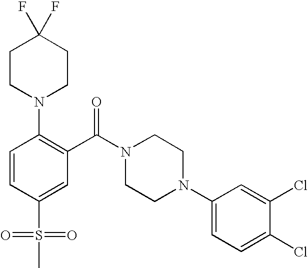 | 1-(3,4-dichlorophenyl)-4-{[2-(4,4-difluoro- 1-piperidinyl)-5- (methylsulfonyl)phenyl]carbonyl}piperazine | 532/534 (M + 1) |
| 52 | 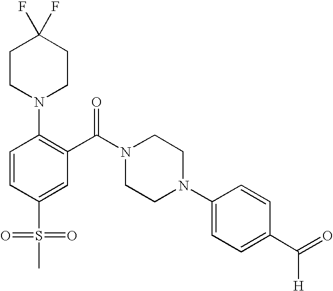 | 4-(4-{[2-(4,4-difluoro-1-piperidinyl)-5- (methylsulfonyl)phenyl]carbonyl}-1- piperazinyl)benzaldehyde | 510 (M + 1) |
| 53 | 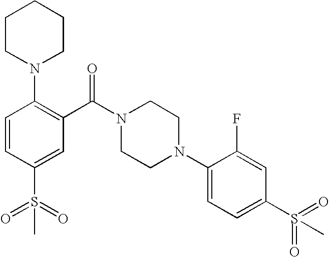 | 1-[2-fluoro-4-(methylsulfonyl)phenyl]-4-{[5- (methylsulfonyl)-2-(1- piperidinyl)phenyl]carbonyl}piperazine | 524 (M + 1) |

The following compound was prepared according to the procedure of Example 37.

| Example | | | LC/MS |
|---|---|---|---|
| No. | Structure | Name | (ESI) |
| 54 | 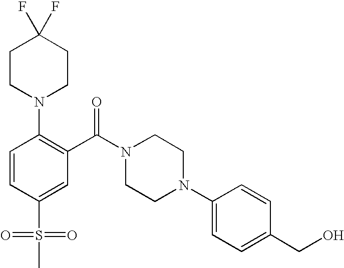 | [4-(4-{[2-(4,4-difluoro-1- piperidinyl)-5- (methylsulfonyl)phenyl]carbonyl}- 1-piperazinyl)phenyl]methanol | 512 (M + 1) |

### EXAMPLE 55

### 1-{[2-(4,4-difluoro-1-piperidinyl)-5-(methylsulfonyl)phenyl]carbonyl}-4-{2-fluoro-4-[(methyloxy)methyl]phenyl}piperazine

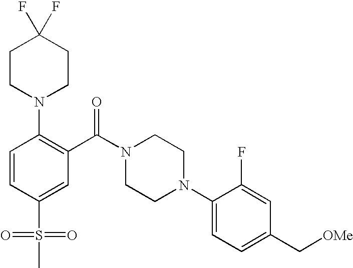

[4-(4-{[2-(4,4-difluoro-1-piperidinyl)-5-(methylsulfonyl)phenyl]carbonyl}-1-piperazinyl)phenyl]methanol, p-toluenesulfonic acid monohydrate and methanol were dissolved in toluene and heated to 80° C. for 6 h with stirring under an atmosphere of argon. After cooling, the solution was evaporated and the residue partitioned between dichloromethane and saturated sodium hydrogen carbonate solution. The organic solution was washed with water and brine and dried (MgSO4). After filtration and evaporation, the residue was chromatographed over silica gel. Elution with a gradient of 0 to 100% ethyl acetate in hexane gave the title compound. LC/MS (ESI) Found 526 (M+1).

### EXAMPLE 56

### 1-(4-{[(2,2-difluoroethyl)oxy]methyl}-2-fluorophenyl)-4-{[2-(4,4-difluoro-1-piperidinyl)-5-(methylsulfonyl)phenyl]carbonyl}piperazine

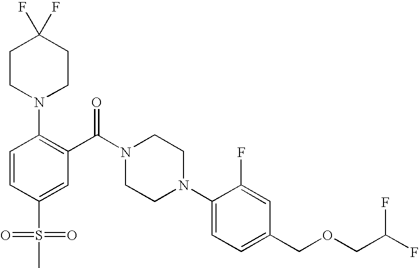

The title compound was prepared from [4-(4-{[2-(4,4-difluoro-1-piperidinyl)-5-(methylsulfonyl)phenyl]carbonyl}-1-piperazinyl)phenyl]methanol, p-toluenesulfonic acid monohydrate and 2,2-difluoroethanol by the method of Example 55. LC/MS (ESI) Found 576 (M+1).

### EXAMPLE 57

### 1-[3,5-Dichloro-4-(methyloxy)phenyl]-4-{[2-(4,4-difluoro-1-piperidinyl)-5-(methylsulfonyl)phenyl]carbonyl}piperazine

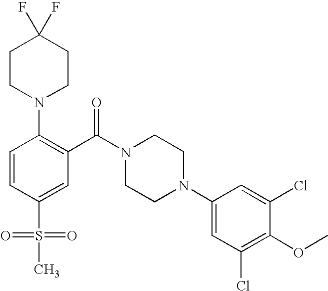

2-(4,4-difluoro-1-piperidinyl)-5-(methylsulfonyl)benzoic acid (0.120 g) and 1-[3,5-dichloro-4-(methyloxy)phenyl]piperazine hydrochloride (0.111 g) were dissolved in DMF (5 ml) and stirred at room temperature for 16 hours, under an argon atmosphere, with an excess of DIPEA (0.4 ml) and HATU (0.143 g). The reaction mixture was then partitioned between ethyl acetate and water. The organic solution was dried (Na₂SO₄) and evaporated to yield the crude product, which was purified by silica gel column chromatography, eluting with a gradient of 0 to 100% ethyl acetate in N-pentane. The title compound was obtained as a white solid (81% yield), LC/MS (ESI) Found 563 (M+1).

### EXAMPLE 58

### 6-(4-{[5-(methylsulfonyl)-2-(1-piperidinyl)phenyl]carbonyl}-1-piperazinyl)-3-pyridinecarbonitrile

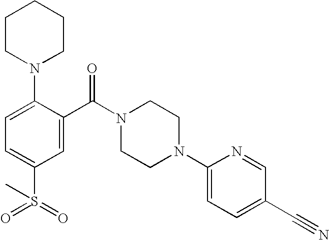

5-Methanesulfonyl-2-piperidin-1-yl-benzoic acid and 6-piperazin-1-ylnicotinonitrile were dissolved in MDC and stirred at room temperature for 16 hours, under an argon atmosphere, with EDC, HOBT and triethylamine. The reaction solution was then evaporated and the residue partitioned between ethyl acetate and water. The organic solution was dried over anhydrous magnesium sulphate, filtered and concentrated at reduced pressure to yield the crude product, which was purified by silica gel column chromatography. Elution with a gradient of 0 to 100% ethyl acetate in pentane provided the title compound. LC/MS (ESI) Found 476 (M+Na)

The following compounds were prepared according to Example 58 using the appropriate benzoic acid derivative and the appropriate piperazine.

| No | Structure | Name | MS |
|---|---|---|---|
| 59 | 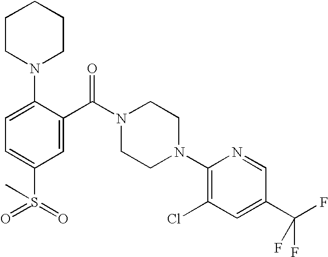 | 1-[3-chloro-5-(trifluoromethyl)-2- pyridinyl]-4-{[5-(methylsulfonyl)-2-(1- piperidinyl)phenyl]carbonyl}piperazine | 531 (M + H) |
| 60 | 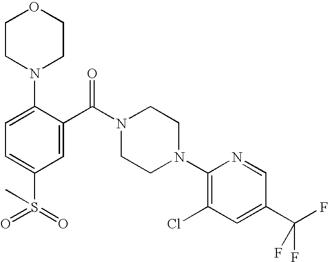 | 4-[2-({4-[3-chloro-5-(trifluoromethyl)-2- pyridinyl]-1-piperazinyl}carbonyl)-4- (methylsulfonyl)phenyl]morpholine | 533 (M + H) |
| 61 | 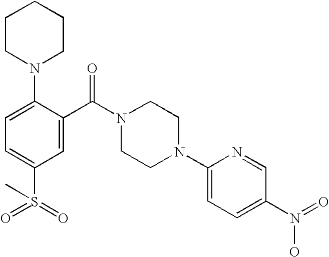 | 1-{[5-(methylsulfonyl)-2-(1- piperidinyl)phenyl]carbonyl}-4-(5-nitro-2- pyridinyl)piperazine | 474 (M + H) |

### EXAMPLE 62

### 6-(4-{[5-(Methylsulfonyl)-2-(1-piperidinyl)phenyl]carbonyl}-1-piperazinyl)-3-pyridinamine

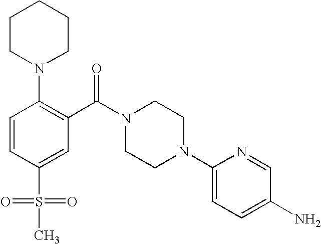

1-{[5-(Methylsulfonyl)-2-(1-piperidinyl)phenyl]carbonyl}-4-(5-nitro-2-pyridinyl)piperazine (Example 107, 350 mg) was dissolved in DCM and ethanol. 10% Palladium on carbon (40 mg) was added and the reaction mixture was shaken under an atmosphere of hydrogen for 21 hours. The catalyst removed by filtration through Kieselguhr, washing well with ethanol. The solvent removed in vacuo to afford the title compound as a brown gummy solid in 45% yield.

LCMS (ES+) 444 (M+H)

### EXAMPLE 63

### N-Acetyl-N-[6-(4-{[5-(methylsulfonyl)-2-(1-piperidinyl)phenyl]carbonyl}-1-piperazinyl)-3-pyridinyl]acetamide

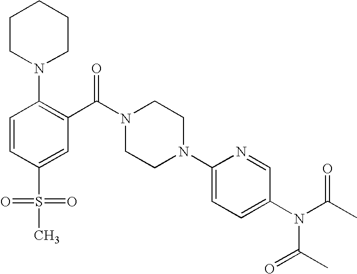

6-(4-{[5-(Methylsulfonyl)-2-(1-piperidinyl)phenyl]carbonyl}-1-piperazinyl)-3-pyridinamine (Example 110, 175 mg) was dissolved in DCM and treated with triethylamine and acetyl chloride. The reaction was stirred for 18 hours at rt, under argon. The reaction diluted with DCM, washed with saturated NaHCO₃ solution and dried over anhydrous magnesium sulphate. The solution was filtered and the solvent removed in vacuo. The residue was purified on silica gel chromatography, eluting with pentane to 5% methanol/ethyl acetate. The desired fractions were collected and solvent evaporated to dryness in vacuo to afford the title compound in 21% yield.

LCMS (ES+) 528 (M+H)

1-23. (canceled)

24. A compound of formula (I) or a salt thereof: 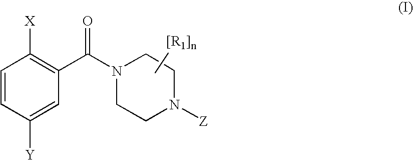
wherein
X is —NR₃R₄, wherein
R₃ and R₄ are independently selected from the group consisting of hydrogen and C₁₋₆alkyl, or R₃ and R₄, together with the nitrogen atom to which they are attached, form an N-linked 3- to 7-membered monocyclic heterocyclic ring or an 8- to 11-membered bicyclic heterocyclic ring, which ring at least one heteroatom selected from the group consisting of N, O and S; and which C₁₋₆alkyl group or ring is unsubstituted or substituted by one or more groups selected from the group consisting of halo, C₁₋₄alkyl, C₁₋₄haloalkyl, C₁₋₄alkoxy, C₁₋₄haloalkoxy, C₁₋₄alkylthio, halo and hydroxy;
Y is S(O)ₘR₅ or —SO₂NHR₆ wherein
m is 1 or 2; and
R₅ is selected from the group consisting of C₁₋₆alkyl, C₃₋₇cycloalkyl, C₅₋₁₁aryl and C₄₋₁₀heteroaryl, wherein each C₁₋₆alkyl, C₃₋₇cycloalkyl, C₅₋₁₁aryl or C₄₋₁₀heteroaryl is unsubstituted or substituted with one or two groups selected from the group consisting of halo, C₁₋₄alkoxy and C₁₋₄haloalkoxy;
R₆ is C₁₋₆alkyl which is unsubstituted or substituted with one or more groups selected from the group consisting of halo, C₁₋₄alkoxy and C₁₋₄haloalkoxy;
n in [R₁]ₙ is 0, 1 or 2,
each R₁ is independently selected from the group consisting of C₁₋₆alkyl, halo, C₁₋₆haloalkyl C₁₋₄alkoxy and C₁₋₄haloalkoxy;
Z is an unsubstituted or substituted phenyl group Z′: 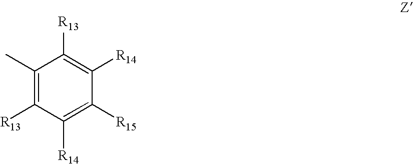
each R₁₃ is independently selected from the group consisting of hydrogen, halogen, hydroxy, cyano, amino, nitro, C₁₋₆alkyl, haloC₁₋₄alkyl, haloC₁₋₄alkoxy, C₆₋₁₁arylC₁₋₄alkoxy, C₁₋₄alkylthio, hydroxyC₁₋₄alkyl, C₁₋₄alkoxyC₁₋₄alkyl, C₃₋₆cycloalkylC₁₋₄alkyl, C₃₋₆cycloalkyl, C₃₋₆cycloalkylC₁₋₄alkoxy, C₁₋₄alkanoyl, C₁₋₄alkoxycarbonyl, C₁₋₄alkoxycarbonylC₁₋₄alkyl, C₁₋₄alkylsulfonyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylamido, C₁₋₄alkylsulfonamidoC₁₋₄alkyl, C₁₋₄alkylamidoC₁₋₄alkyl, C₆₋₁₁arylsulfonamido, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, C₁₋₄acyl, C₆₋₁₁arylC₁₋₄alkyl, C₁₋₄alkylaminoC₁₋₄alkyl, a group R_{9″}R_{10″}N—, R₉R₁₀NCO(CH₂)ₚ, R_{9′}R_{10′}NSO₂(CH₂)ₚ or R_{9′}SO₂NR_{10′}(CH₂)ₚ, —CR_{9′}═NR_{10′}, —CR_{9′}═NOR_{10′}, —CR_{9′}═C(CN)₂, —CR_{9′}═CH(CN), R_{9′}R_{10′}N(CH₂)_{q}— and R_{9′}R_{10′}N(CH₂)_{q}O—, wherein
each R₉ and R₁₀ is independently C₁₋₄alkyl, or R₉R₁₀ forms part of a C₃₋₆azacyloalkane or C₃₋₆(2-, 3- or 4-oxo)azacycloalkane ring;
each R_{9′} and R_{10′} is independently selected from the group consisting of R₉ and R₁₀ and hydrogen;
each R_{9″} and R_{10″} is independently selected from the group consisting of R_{9′} and R_{10′} and C₁₋₄alkanoyl;
p is 0, 1, 2, 3 or 4;
q is 2, 3 or 4;
each R₁₄ is independently selected from the group consisting of hydrogen, halogen, hydroxy, cyano, amino, nitro, C₁₋₆alkyl, C₁₋₄alkoxy, haloC₁₋₄alkyl, haloC₁₋₄alkoxy, C₆₋₁₁arylC₁₋₄alkoxy, C₁₋₄alkylthio, C₃₋₆cycloalkylC₁₋₄alkyl, C₃₋₆cycloalkyl, C₃₋₆cycloalkylC₁₋₄alkoxy, C₁₋₄alkoxycarbonyl, C₁₋₄alkoxycarbonylC₁₋₄alkyl, C₁₋₄alkylsulfonyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₁₋₄alkylamido, C₁₋₄alkylsulfonamidoC₁₋₄alkyl, C₁₋₄alkylsulfinyl, C₁₋₄haloalkylsulfinyl, C₁₋₄alkylamidoC₁₋₄alkyl, C₆₋₁₁arylsulfonamido, C₄₋₉heteroarylsulfonyl, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, C₁₋₄acyl, C₆₋₁₁aryl, C₆₋₁₁arylC₁₋₄alkyl, C₁₋₄alkylaminoC₁₋₄alkyl, a group R_{9″}R_{10″}N—, R₉R₁₀NCO(CH₂)ₚ, R_{9′}R_{10′}NSO₂(CH₂)ₚ or R_{9′}SO₂NR_{10′}(CH₂)ₚ, —CR_{9′}═NR_{10′}, —CR_{9′}═NOR_{10′}, —CR_{9′}═C(CN)₂, —CR_{9′}═CH(CN), R_{9′}R_{10′}N(CH₂)_{q}— and R_{9′}R_{10′}N(CH₂)_{q}O—, wherein
each R₉ and R₁₀ is independently C₁₋₄alkyl, or R₉R₁₀ forms part of a C₃₋₆azacyloalkane or C₃₋₆(2-, 3- or 4-oxo)azacycloalkane ring
each R_{9′} and R_{10′} is independently selected from the group consisting of R₉ and R₁₀ and hydrogen;
each R_{9″} and R_{10″} is independently selected from the group consisting of R_{9′} and R_{10′} and C₁₋₄alkanoyl;
p is 0, 1, 2, 3 or 4;
q is 2, 3 or 4;
each R₁₅ is independently selected from the group consisting of hydrogen, halogen, hydroxy, cyano, amino, nitro, C₁₋₆alkyl, C₁₋₄alkoxy, haloC₁₋₄alkyl, haloC₁₋₄alkoxy, C₆₋₁₁arylC₁₋₄alkoxy, C₁₋₄alkylthio, hydroxyC₁₋₄alkyl, C₁₋₄alkoxyC₁₋₄alkyl, C₁₋₄haloalkoxyC₁₋₄alkyl, C₃₋₆cycloalkylC₁₋₄alkyl, C₃₋₆cycloalkyl, C₃₋₆cycloalkylC₁₋₄alkoxy, C₁₋₄alkanoyl, C₁₋₄haloalkanoyl, C₁₋₄alkoxycarbonyl, C₁₋₄alkoxycarbonylC₁₋₄alkyl, C₁₋₄alkylsulfonyl, C₁₋₄haloalkylsulfonyl, C₁₋₄alkylsulfinyl, C₁₋₄haloalkylsulfinyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylamido, C₁₋₄alkylsulfonamidoC₁₋₄alkyl, C₁₋₄alkylamidoC₁₋₄alkyl, C₆₋₁₁arylsulfonamido, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, C₁₋₄acyl, C₆₋₁₁aryl, C₆₋₁₁arylC₁₋₄alkyl, C₁₋₄alkylaminoC₁₋₄alkyl, a group R_{9″}R_{10″}N—, R₉R₁₀NCO(CH₂)ₚ, R_{9′}R_{10′}NSO₂(CH₂)ₚ or R_{9′}SO₂NR_{10′}(CH₂)ₚ, —CR_{9′}═NR_{10′}, —CR_{9′}═NOR_{10′}, —CR_{9′}═C(CN)₂, —CR_{9′}═CH(CN), R_{9′}R_{10′}N(CH₂)_{q}— and R_{9′}R_{10′}N(CH₂)_{q}O—, wherein
each R₉ and R₁₀ is independently C₁₋₄alkyl, or R₉R₁₀ forms part of a C₃₋₆azacyloalkane or C₃₋₆(2-, 3- or 4-oxo)azacycloalkane ring
each R_{9′} and R_{10′} is independently selected from the group consisting of R₉ and R₁₀ and hydrogen;
each R_{9″} and R_{10″} is independently selected from the group consisting of R_{9′} and R_{10′} and C₁₋₄alkanoyl;
p is 0, 1, 2, 3 or 4;
q is 2, 3 or 4;
or Z is a monocyclic or bicyclic heteroaryl group, wherein the monocyclic heteroaryl group is the bicyclic heteroaryl group is unsubstituted or substituted by one or more groups selected from the group consisting of amino, halogen, hydroxy, cyano, nitro, C₂₋₄alkyl, C₁₋₄alkoxy, haloC₁₋₄alkyl, haloC₁₋₄alkoxy, C₆₋₁₁arylC₁₋₄alkoxy, C₁₋₄alkylthio, hydroxyC₁₋₄alkyl, C₁₋₄alkoxyC₁₋₄alkyl, C₁₋₄haloalkoxyC₁₋₄alkyl, C₃₋₆cycloalkylC₁₋₄alkyl, C₃₋₆cycloalkyl, C₃₋₆cycloalkylC₁₋₄alkoxy, C₁₋₄alkanoyl, C₁₋₄haloalkanoyl, C₁₋₄alkoxycarbonyl, C₁₋₄alkoxycarbonylC₁₋₄alkyl, C₁₋₄alkylsulfonyl, C₁₋₄haloalkylsulfonyl, C₁₋₄alkylsulfinyl, C₁₋₄haloalkylsulfinyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylamido, C₁₋₄alkylsulfonamidoC₁₋₄alkyl, C₁₋₄alkylamidoC₁₋₄alkyl, C₆₋₁₁arylsulfonamido, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, C₁₋₄acyl, C₆₋₁₁aryl, C₆₋₁₁arylC₁₋₄alkyl, C₁₋₄alkylaminoC₁₋₄alkyl, a group R_{9″}R_{10″}N—, R₉R₁₀NCO(CH₂)ₚ, R_{9′}R_{10′}NSO₂(CH₂)ₚ or R_{9′}SO₂NR_{10′}(CH₂)ₚ, —CR_{9′}═NR_{10′}, —CR_{9′}═NOR_{10′}, —CR_{9′}═C(CN)₂, —CR_{9′}═CH(CN), R_{9′}R_{10′}N(CH₂)_{q}— and R_{9′}R_{10′}N(CH₂)_{q}O—, wherein
each R₉ and R₁₀ is independently C₁₋₄alkyl, or R₉R₁₀ forms part of a C₃₋₆azacyloalkane or C₃₋₆(2-, 3- or 4-oxo)azacycloalkane ring
each R_{9′} and R_{10′} is independently selected from the group consisting of R₉ and R₁₀ and hydrogen;
each R_{9″} and R_{10″} is independently selected from the group consisting of R_{9′} and R_{10′} and C₁₋₄alkanoyl;
p is 0, 1, 2, 3 or 4; and
q is 2, 3 or 4.

25. A compound according to claim 24 wherein X is —NR₃R₄; and R₃ and R₄ are independently selected from the group consisting of hydrogen and C₁₋₆alkyl, which C₁₋₆alkyl group is unsubstituted or substituted by one or more groups selected from the group consisting of halo, C₁₋₄alkyl, C₁₋₄haloalkyl, C₁₋₄alkoxy, C₁₋₄haloalkoxy, C₁₋₄alkylthio, halo and hydroxy.

26. A compound according to claim 24 wherein X is —NR₃R₄; and R₃ and R₄, together with the nitrogen atom to which they are attached, form an N-linked 3- to 7-membered monocyclic heterocyclic ring or an 8- to 11-membered bicyclic heterocyclic ring, which ring optionally has one or more further hetero atoms selected from the group consisting of N, O and S; and which ring is unsubstituted or substituted by one or more groups selected from the group consisting of halo, C₁₋₄alkyl, C₁₋₄haloalkyl, C₁₋₄alkoxy, C₁₋₄haloalkoxy, C₁₋₄alkylthio, halo and hydroxyl.

27. A compound according to claim 26 wherein R₃ and R₄, together with the nitrogen to which they are attached, form a morpholinyl, piperidinyl or azepanyl group, unsubstituted or substituted by one or more groups selected from the group consisting of halo, C₁₋₄alkyl, C₁₋₄haloalkyl, C₁₋₄alkoxy, C₁₋₄haloalkoxy, C₁₋₄alkylthio, halo and hydroxyl.

28. A compound according to claim 26 wherein R₃ and R₄, together with the nitrogen to which they are attached, form a 8- to 11-membered bicyclic heterocyclic ring unsubstituted or substituted by one or more groups selected from the group consisting of halo, C₁₋₄alkyl, C₁₋₄haloalkyl, C₁₋₄alkoxy, C₁₋₄haloalkoxy, C₁₋₄alkylthio, halo and hydroxyl.

27. A compound according to claim 24 wherein Y is S(O)₂R₅ and R₅ is C₁₋₆alkyl.

28. A compound according to claim 24 wherein Z is a phenyl group Z′.

29. A compound according to claim 28 wherein each R₁₃ is independently selected from the group consisting of hydrogen, halogen, cyano and C₁₋₄alkoxyC₁₋₄alkyl.

30. A compound according to claim 29 wherein each R₁₄ is independently selected from the group consisting of hydrogen, halogen, cyano, nitro, C₁₋₆alkyl, C₁₋₄alkoxy and haloC₁₋₄alkyl.

31. A compound according to claim 29 wherein each R₁₅ is independently selected from the group consisting of hydrogen, halogen, cyano, amino, nitro, C₁₋₆alkyl, C₁₋₄alkoxy, haloC₁₋₄alkyl, C₁₋₄alkylthio, hydroxyC₁₋₄alkyl, C₁₋₄alkoxyC₁₋₄alkyl, C₁₋₄haloalkoxyC₁₋₄alkyl, C₁₋₄alkanoyl, C₁₋₄haloalkanoyl, C₁₋₄alkylsulfonyl, C₁₋₄alkylsulfinyl, C₁₋₄haloalkylsulfinyl, R₉R₁₀NCO(CH₂)ₚ, —CR_{9′}═NR_{10′}, and —CR_{9′}═NOR_{10′}, wherein
each R₉ and R₁₀ is independently C₁₋₄alkyl, or R₉R₁₀ forms part of a C₃₋₆azacyloalkane or C₃₋₆(2-, 3- or 4-oxo)azacycloalkane ring;
each R_{9′} and R_{10′} is independently selected from the group consisting of R₉ and R₁₀ and hydrogen; and
p is selected from 0, 1, 2, 3 or 4.

32. A compound according to claim 24 wherein Z is selected from the group consisting of pyridyl, pyrimidinyl, 1H-pyrrolo[2,3-b]pyridinyl, pyridazinyl, pyrazinyl, triazolyl, triazinyl, pyrrolyl, imidazolyl, thienyl, furanyl, thiadiazolyl, isoxazolyl, isothiazolyl, thiazolyl, oxadiazolyl and oxazolyl, benzothiazolyl, 1,4-benzodioxinyl, 2,3-dihydro-1,4-benzodioxinyl, benzoxazolyl, indolyl, quinolyl, isoquinolinyl, 1-benzopyranyl, 2-benzopyranyl, dihyrdo-1-benzopyranyl, dihydro-2-benzopyranyl, quinoxalinyl and quinazolinyl, each of which is unsubstituted, or substituted by one or more groups selected from the group consisting of amino, halogen, hydroxy, cyano, nitro, C₂₋₄alkyl, C₁₋₄alkoxy, haloC₁₋₄alkyl, haloC₁₋₄alkoxy, C₆₋₁₁arylC₁₋₄alkoxy, C₁₋₄alkylthio, hydroxyC₁₋₄alkyl, C₁₋₄alkoxyC₁₋₄alkyl, C₁₋₄haloalkoxyC₁₋₄alkyl, C₃₋₆cycloalkylC₁₋₄alkyl, C₃₋₆cycloalkyl, C₃₋₆cycloalkylC₁₋₄alkoxy, C₁₋₄alkanoyl, C₁₋₄haloalkanoyl, C₁₋₄alkoxycarbonyl, C₁₋₄alkoxycarbonylC₁₋₄alkyl, C₁₋₄alkylsulfonyl, C₁₋₄haloalkylsulfonyl, C₁₋₄alkylsulfinyl, C₁₋₄haloalkylsulfinyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylamido, C₁₋₄alkylsulfonamidoC₁₋₄alkyl, C₁₋₄alkylamidoC₁₋₄alkyl, C₆₋₁₁arylsulfonamido, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, C₁₋₄acyl, C₆₋₁₁aryl, C₆₋₁₁arylC₁₋₄alkyl, C₁₋₄alkylaminoC₁₋₄alkyl, a group R_{9″}R_{10″}N—, R₉R₁₀NCO(CH₂)ₚ, R_{9′}R_{10′}NSO₂(CH₂)ₚ or R_{9′}SO₂NR_{10′}(CH₂)ₚ, —CR_{9′}═NR_{10′}, —CR_{9′}═NOR_{10′}, —CR_{9′}═C(CN)₂, —CR_{9′}═CH(CN), R_{9′}R_{10′}N(CH₂)_{q}— and R_{9′}R_{10′}N(CH₂)_{q}O—, wherein
each R₉ and R₁₀ is independently C₁₋₄alkyl, or R₉R₁₀ forms part of a C₃₋₆azacyloalkane or C₃₋₆(2-, 3- or 4-oxo)azacycloalkane ring
each R_{9′} and R_{10′} is independently selected from the group consisting of R₉ and R₁₀ and hydrogen;
each R_{9″} and R_{10″} is independently selected from the group consisting of R_{9′} and R_{10′} and C₁₋₄alkanoyl;
p is 0, 1, 2, 3 or 4; and
q is 2, 3 or 4.

33. A compound according to claim 32 in which the group Z is selected from the group consisting of pyrid-2-yl, pyrimidin-2-yl, pyrimidin-4-yl, quinoxalinyl, quinolinyl and 1H-pyrrolo[2,3-b]pyridinyl.

34. A compound according to claim 33 which is unsubstituted or substituted with one or more groups selected from the group consisting of amino, cyano, nitro, haloC₁₋₄alkyl, C₁₋₄alkanoyl, hydroxyC₁₋₄alkyl, C₁₋₄alkoxy, C₁₋₄alkoxycarbonyl, C₄₋₉heteroarylsulfonyl, and R_{9″}R_{10″}N—, wherein each R_{9″} and R_{10″} is independently selected from the group consisting of hydrogen, C₁₋₄alkyl and C₁₋₄alkanoyl.

35. A compound as claimed in claim 24, which is Example 1 to 63, or a salt thereof.

36. A method of preparing a compound of formula (I) as defined in claim 24 or a salt or solvate thereof, comprising the step of:
(a) reacting a compound of formula (II): 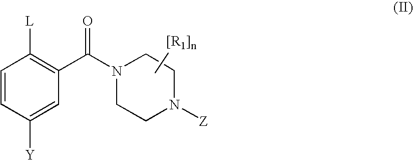
wherein L is a leaving group such as halogen or triflate, and Y. R₁, n and Z are as defined in claim 24, with a compound of formula (III):
H—X (III)
wherein X is as defined in claim 24 and H is hydrogen; or
(b) reacting a compound of formula (IV): 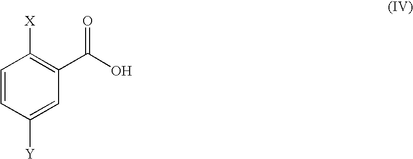
wherein X and Y are as defined in claim 24, with a compound of formula (V): 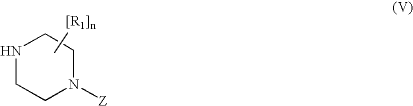
wherein R₁, n and Z are as defined in claim 24; or
(c) reacting a compound of formula (VI): 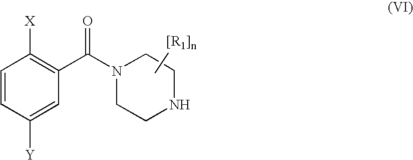
wherein X, Y, n and R₁ are as defined for formula (I), with a group Z-L wherein Z is as defined for formula (I) and L is a leaving group, under basic conditions with a suitable catalyst, and a suitable ligand; or by heating a compound of formula (VI) with a group Z-L to 180° C., with or without diisopropylamine as solvent, in a microwave reactor;
and thereafter optionally for step (a), step (b) or step (c),
removing any protecting groups and/or
converting a compound of formula (I) into another compound of formula (I) and/or
forming a salt or solvate.

37. A pharmaceutical composition comprising a compound according to claim 24 and at least one pharmaceutically acceptable carrier, diluent or excipient.

38. A method of treating schizophrenia in a human which comprises administering an effective amount of a compound according to claim 24 or a salt thereof

39. A method of treating schizophrenia in a human which comprises administering an effective amount of a compound of formula (Ib) or a salt: 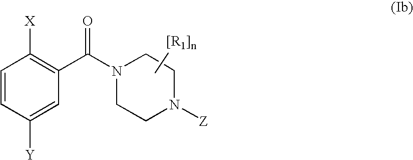
wherein
X is —NR₃R₄, wherein
R₃ and R₄ are independently selected from the group consisting of hydrogen and C₁₋₆alkyl, or R₃ and R₄, together with the nitrogen atom to which they are attached, form an N-linked 3- to 7-membered monocyclic heterocyclic ring or an 8- to 11-membered bicyclic heterocyclic ring, containing one or more further heteroatoms selected from the group consisting of N, O and S; and the C₁₋₆alkyl group or ring is unsubstituted or substituted by one or more groups selected from the group consisting of halo, C₁₋₄alkyl, C₁₋₄haloalkyl, C₁₋₄alkoxy, C₁₋₄haloalkoxy, C₁₋₄alkylthio, halo and hydroxy;
Y is S(O)ₘR₅ or —SO₂NR₆R₇ wherein
m is 1 or 2; and
R₅ is selected from the group consisting of C₁₋₆alkyl, C₃₋₇cycloalkyl, C₅₋₁₁aryl and C₄₋₁₀heteroaryl, wherein C₁₋₆alkyl, C₃₋₇cycloalkyl, C₅₋₁₁aryl or C₄₋₁₀heteroaryl is unsubstituted or substituted with one or two groups selected from the group consisting of halo, C₁₋₄alkoxy and C₁₋₄haloalkoxy;
R₆ and R₇ are independently selected from the group consisting of hydrogen and C₁₋₆alkyl but are not both simultaneously C₁₋₆alkyl; wherein the C₁₋₆alkyl is unsubstituted or substituted with one or more groups selected from the group consisting of halo, C₁₋₄alkoxy and C₁₋₄haloalkoxy;
n is 0, 1 or 2,
each R₁ is independently selected from the group consisting of C₁₋₆alkyl, halo, C₁₋₆haloalkyl C₁₋₄alkoxy and C₁₋₄haloalkoxy;
Z is an unsubstituted or substituted phenyl group Z′: 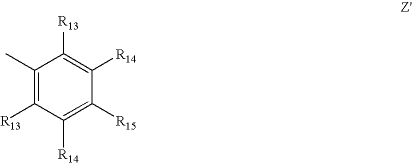
each R₁₃ is independently selected from the group consisting of hydrogen, halogen, hydroxy, cyano, amino, nitro, C₁₋₆alkyl, haloC₁₋₄alkyl, haloC₁₋₄alkoxy, C₆₋₁₁arylC₁₋₄alkoxy, C₁₋₄alkylthio, hydroxyC₁₋₄alkyl, C₁₋₄alkoxyC₁₋₄alkyl, C₃₋₆cycloalkylC₁₋₄alkyl, C₃₋₆cycloalkyl, C₃₋₆cycloalkylC₁₋₄alkoxy, C₁₋₄alkanoyl, C₁₋₄alkoxycarbonyl, C₁₋₄alkoxycarbonylC₁₋₄alkyl, C₁₋₄alkylsulfonyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylamido, C₁₋₄alkylsulfonamidoC₁₋₄alkyl, C₁₋₄alkylamidoC₁₋₄alkyl, C₆₋₁₁arylsulfonamido, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, C₁₋₄acyl, C₆₋₁₁arylC₁₋₄alkyl, C₁₋₄alkylaminoC₁₋₄alkyl, a group R_{9″}R_{10″}N—, R₉R₁₀NCO(CH₂)ₚ, R_{9′}R_{10′}NSO₂(CH₂)ₚ or R_{9′}SO₂NR_{10′}(CH₂)ₚ, —CR_{9′}═NR_{10′}, —CR_{9′}═NOR_{10′}, —CR_{9′}═C(CN)₂, —CR_{9′}═CH(CN), R_{9′}R_{10′}N(CH₂)_{q}— and R_{9′}R_{10′}N(CH₂)_{q}O—, wherein
each R₉ and R₁₀ is independently C₁₋₄alkyl, or R₉R₁₀ forms part of a C₃₋₆azacyloalkane or C₃₋₆(2-, 3- or 4-oxo)azacycloalkane ring
each R_{9′} and R_{10′} is independently selected from the group consisting of R₉ and R₁₀ and hydrogen;
each R_{9″} and R_{10″} is independently selected from the group consisting of R_{9′} and R_{10′} and C₁₋₄alkanoyl;
p is 0, 1, 2, 3 or 4;
q is 2, 3 or 4;
each R₁₄ is independently selected from the group consisting of hydrogen, halogen, hydroxy, cyano, amino, nitro, C₁₋₆alkyl, C₁₋₄alkoxy, haloC₁₋₄alkyl, haloC₁₋₄, alkoxy, C₆₋₁₁arylC₁₋₄alkoxy, C₁₋₄alkylthio, C₃₋₆cycloalkylC₁₋₄alkyl, C₃₋₆cycloalkyl, C₃₋₆cycloalkylC₁₋₄alkoxy, C₁₋₄alkoxycarbonyl, C₁₋₄alkoxycarbonylC₁₋₄alkyl, C₁₋₄alkylsulfonyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₁₋₄alkylamido, C₁₋₄alkylsulfonamidoC₁₋₄alkyl, C₁₋₄alkylsulfinyl, C₁₋₄haloalkylsulfinyl, C₁₋₄alkylamidoC₁₋₄alkyl, C₆₋₁₁arylsulfonamido, C₄₋₉heteroarylsulfonyl, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, C₁₋₄acyl, C₆₋₁₁aryl, C₆₋₁₁arylC₁₋₄alkyl, C₁₋₄alkylaminoC₁₋₄alkyl, a group R_{9″}R_{10″}N—, R₉R₁₀NCO(CH₂)ₚ, R_{9′}R_{10′}NSO₂(CH₂)ₚ or R_{9′}SO₂NR_{10′}(CH₂)ₚ, —CR_{9′}═NR_{10′}, —CR_{9′}═NOR_{10′}, —CR_{9′}═C(CN)₂, —CR_{9′}═CH(CN), R_{9′}R_{10′}N(CH₂)_{q}— and R_{9′}R_{10′}N(CH₂)_{q}O—, wherein
each R₉ and R₁₀ is independently C₁₋₄alkyl, or R₉R₁₀ forms part of a C₃₋₆azacyloalkane or C₃₋₆(2-, 3- or 4-oxo)azacycloalkane ring
each R_{9′} and R_{10′} is independently selected from the group consisting of R₉ and R₁₀ and hydrogen;
each R_{9″} and R_{10′} is independently selected from the group consisting of R_{9′} and R_{10′} and C₁₋₄alkanoyl;
p is 0, 1, 2, 3 or 4;
q is 2, 3 or 4;
each R₁₅ is independently selected from the group consisting of hydrogen, halogen, hydroxy, cyano, amino, nitro, C₁₋₆alkyl, C₁₋₄alkoxy, haloC₁₋₄alkyl, haloC₁₋₄alkoxy, C₆₋₁₁arylC₁₋₄alkoxy, C₁₋₄alkylthio, hydroxyC₁₋₄alkyl, C₁₋₄alkoxyC₁₋₄alkyl, C₁₋₄haloalkoxyC₁₋₄alkyl, C₃₋₆cycloalkylC₁₋₄alkyl, C₃₋₆cycloalkyl, C₃₋₆cycloalkylC₁₋₄alkoxy, C₁₋₄alkanoyl, C₁₋₄haloalkanoyl, C₁₋₄alkoxycarbonyl, C₁₋₄alkoxycarbonylC₁₋₄alkyl, C₁₋₄alkylsulfonyl, C₁₋₄haloalkylsulfonyl, C₁₋₄alkylsulfinyl, C₁₋₄haloalkylsulfinyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylamido, C₁₋₄alkylsulfonamidoC₁₋₄alkyl, C₁₋₄alkylamidoC₁₋₄alkyl, C₆₋₁₁arylsulfonamido, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, C₁₋₄acyl, C₆₋₁₁aryl, C₆₋₁₁arylC₁₋₄alkyl, C₁₋₄alkylaminoC₁₋₄alkyl, a group R_{9″}R_{10″}N—, R₉R₁₀NCO(CH₂)ₚ, R_{9′}R_{10′}NSO₂(CH₂)ₚ or R_{9′}SO₂NR_{10′}(CH₂)ₚ, —CR_{9′}═NR_{10′}, —CR_{9′}═NOR_{10′}, —CR_{9′}═C(CN)₂, —CR_{9′}═CH(CN), R_{9′}R_{10′}N(CH₂)_{q}— and R_{9′}R_{10′}N(CH₂)_{q}O—, wherein
each R₉ and R₁₀ is independently C₁₋₄alkyl, or R₉R₁₀ forms part of a C₃₋₆azacyloalkane or C₃₋₆(2-, 3- or 4-oxo)azacycloalkane ring
each R_{9′} and R_{10′} is independently selected from the group consisting of R₉ and R₁₀ and hydrogen;
each R_{9″} and R_{10″} is independently selected from the group consisting of R_{9′} and R_{10′} and C₁₋₄alkanoyl;
p is 0, 1, 2, 3 or 4;
q is 2, 3 or 4;
or Z is selected from the group consisting of a monocyclic or bicyclic heteroaryl group, which monocyclic heteroaryl group is or which bicyclic heteroaryl group optionally is substituted by one or more groups selected from amino, halogen, hydroxy, cyano, nitro, C₂₋₄alkyl, C₁₋₄alkoxy, haloC₁₋₄alkyl, haloC₁₋₄alkoxy, C₆₋₁₁arylC₁₋₄alkoxy, C₁₋₄alkylthio, hydroxyC₁₋₄alkyl, C₁₋₄alkoxyC₁₋₄alkyl, C₁₋₄haloalkoxyC₁₋₄alkyl, C₃₋₆cycloalkylC₁₋₄alkyl, C₃₋₆cycloalkyl, C₃₋₆cycloalkylC₁₋₄alkoxy, C₁₋₄alkanoyl, C₁₋₄haloalkanoyl, C₁₋₄alkoxycarbonyl, C₁₋₄alkoxycarbonylC₁₋₄alkyl, C₁₋₄alkylsulfonyl, C₁₋₄haloalkylsulfonyl, C₁₋₄alkylsulfinyl, C₁₋₄haloalkylsulfinyl, C₁₋₄alkylsulfonyloxy, C₁₋₄alkylsulfonylC₁₋₄alkyl, C₆₋₁₁arylsulfonyl, C₆₋₁₁arylsulfonyloxy, C₆₋₁₁arylsulfonylC₁₋₄alkyl, C₁₋₄alkylsulfonamido, C₄₋₉heteroarylsulfonyl, C₁₋₄alkylamido, C₁₋₄alkylsulfonamidoC₁₋₄alkyl, C₁₋₄alkylamidoC₁₋₄alkyl, C₆₋₁₁arylsulfonamido, C₆₋₁₁arylcarboxamido, C₆₋₁₁arylsulfonamidoC₁₋₄alkyl, C₆₋₁₁arylcarboxamidoC₁₋₄alkyl, C₆₋₁₁aroyl, C₆₋₁₁aroylC₁₋₄alkyl, C₆₋₁₁arylC₁₋₄alkanoyl, C₁₋₄acyl, C₆₋₁₁aryl, C₆₋₁₁arylC₁₋₄alkyl, C₁₋₄alkylaminoC₁₋₄alkyl, a group R_{9″}R_{10′}N—, R₉R₁₀NCO(CH₂)ₚ, R_{9′}R_{10′}NSO₂(CH₂)ₚ or R_{9′}SO₂NR_{10′}(CH₂)ₚ, —CR_{9′}═NR_{10′}, —CR_{9′}═NOR_{10′}, —CR_{9′}═C(CN)₂, —CR_{9′}═CH(CN), R_{9′}R_{10′}N(CH₂)_{q}— and R_{9′}R_{10′}N(CH₂)_{q}O—, wherein
each R₉ and R₁₀ is independently C₁₋₄alkyl, or R₉R₁₀ forms part of a C₃₋₆azacyloalkane or C₃₋₆(2-, 3- or 4-oxo)azacycloalkane ring;
each R_{9′} and R_{10′} is independently selected from the group consisting of R₉ and R₁₀ and hydrogen;
each R_{9″} and R_{10″} is independently selected from the group consisting of R_{9″} and R_{10′} and C₁₋₄alkanoyl;
p is 0, 1, 2, 3 or 4; and
q is 2, 3 or 4.
